(19) United States
(12) Patent Application Publication
(10) Pub. No.: US 2013/0237507 A1
(43) Pub. Date: Sep. 12, 2013

(54) Novel Amide Compounds

(71) Applicant: ASTRAZENECA AB, INTELLECTUAL PROPERTY, Macclesfield (GB)

(72) Inventors: Frank Burkamp, Cheshire (GB); Balint Gabos, Cheshire (GB); Svetlana Ivanova, Cheshire (GB); Alice Ingrid Annea Lisius, Cheshire (GB)

(21) Appl. No.: 13/864,644

(22) Filed: Apr. 17, 2013

Related U.S. Application Data
(63) Continuation of application No. 12/749,903, filed on Mar. 30, 2010.
(60) Provisional application No. 61/166,324, filed on Apr. 3, 2009.

(51) Int. Cl. C07J 71/00

(52) U.S. Cl. CPC C07J 71/0047
USPC 514/176; 540/52

(57) ABSTRACT

The present invention provides compounds of formula (1)

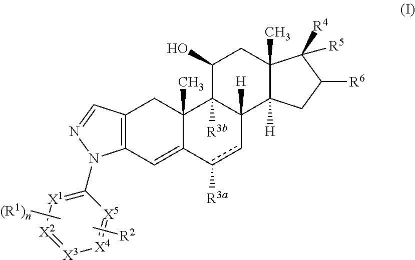

wherein n, R¹, R², X¹, X², X³, X⁴, X⁵, R^{3a}, R^{3b}, R⁴, R⁵ and R⁶ are as defined in the specification, a process for their preparation, pharmaceutical compositions containing them and their use in therapy.

This application claims the benefit of U.S. Provisional application No. 61/166,324, filed Apr. 3, 2009, the disclosure of which is incorporated by reference herein in its entirety.

The present invention relates to compounds having glucocorticosteroid receptor agonist activity, processes for their preparation, pharmaceutical compositions containing them and their therapeutic use, particularly for the treatment of inflammatory and allergic conditions.

Glucocorticosteroids (GCs) that have anti-inflammatory properties are known and are widely used for the treatment of diseases such as inflammatory arthritides (e.g. rheumatoid arthritis, ankylosing spondylitis and psoriatic arthropathy), other rheumatoid diseases such as systemic lupus erythematosis, scleroderma, vascutitides including temporal arteritis and polyarteritis nodosa, inflammatory bowel disease such as Crohns disease and ulcerative colitis, lung diseases such as asthma and chronic obstructive airways disease, as well as many other conditions such as polymyalgia rheumatica. GCs have also been used very extensively for their immunosuppressive properties in the prevention and treatment of transplant rejection. Finally GCs have been used for their anti-tumour effects in a number of malignancies.

GCs act via specific glucocorticoid receptors (GR) that are members of the nuclear receptor superfamily. Ligand binding promotes receptor dimerisation, DNA binding, and transcriptional activation. This mechanism of GC action is well defined in vitro and is critical for regulation of the hypothalamic-pituitary-adrenal axis, gluconeogenesis as well as transcription of anti-inflammatory genes such as mitogen-activated protein kinase phosphatase-1 (MKP-1) and secretory leukocyte protease inhibitor (SLPI) in vivo. Ligand-bound receptor is also able to suppress gene transcription in a dimerisation-independent manner by interfering with the activity of transcription factors, such as AP-1 and NFkB, which are critically involved in the inflammatory reaction.

After ligand binding, the GR translocates from the cytoplasm of the cell to the nucleus and binds to glucocorticoid response elements in regulator regions of target genes. The activated GR then recruits co-factors, including the glucocorticoid receptor interacting protein 1 (GRIP-1) and steroid receptor co-activator 1 (SRC₁). These accessory proteins bind to the receptor and link the GR with the general transcription machinery to drive transcription of target genes.

Glucocorticoid effects on transcription may be mediated by both the direct binding of activated GR to target DNA, homodimerisation and recruitment of co-activators (known as “transactivation”) but also by GR interfering with other transcription factor function, including AP-1 and NFkB, by complexing with these other transcription factors and preventing them from binding to their target genes leading to repression of the genes normally upregulated by AP-1 or NFkB (known as “transrepression”). These two modes of receptor activity are dissociable and negative effects on NFkB activity can be retained in the absence of transactivation. It appears that transrepression is largely responsible for mediating the therapeutically desirable anti-inflammatory activity of the GR. Interestingly, the IC₅₀ for inhibition of AP-1 or NFkB (0.04 nM) is lower than the EC₅₀ for activation of target genes (5 nM) and yet high doses of GCs are frequently required to treat patients with inflammatory disease. One explanation is that cytokines expressed at the site of inflammation may induce relative glucocorticoid resistance, for instance by activating AP-1 or NFkB. This is of importance as many pro-inflammatory cytokines signal by activation of NFkB and a major anti-inflammatory action of GCs is thought to be mediated by opposing NFkB action.

Published Japanese Patent Application No. 60067495 describes certain pregnenopyrazoles as anti-inflammatory agents.

In accordance with the present invention, there is provided a compound of formula

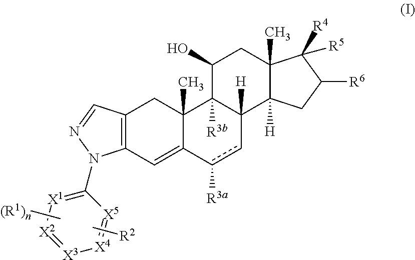

wherein
X¹, X², X³, X⁴ and X⁵ each independently represent CH or a nitrogen atom, provided that no more than two of X¹, X², X³, X⁴ and X⁵ may simultaneously represent a nitrogen atom;
n is 0 or 1;
R¹ represents a halogen atom or a methyl or a methoxy group;
R² represents —C(O)NR⁷R⁸;
R^{3a} represents a hydrogen atom or methyl group and R^{3b} represents a hydrogen or fluorine atom;
R⁴ represents-C(O)—Y—CH(R¹¹)—R⁹ or —C(O)—CH(R¹¹)—Y—R⁹;
R⁵ represents hydroxyl, —OCH₂SCH₃, —O—C(O)—R¹⁰, —O—C(O)—NH—R¹⁰, —O—C(O)—O—R¹⁰ or —O—C(O)—S—R¹⁰;
R⁶ represents a hydrogen or a halogen atom or a hydroxyl or methyl group;
either R⁷ represents a hydrogen atom or a C₁-C₆ alkyl group and R⁸ represents hydrogen, C₁-C₆ alkyl (optionally substituted by cyano, hydroxyl, C₁-C₆ alkoxy, C₁-C₆ haloalkoxy, —NR¹³R¹⁴, —C(O)NR¹³R¹⁴, —NR¹³C(O)C₁-C₆ alkyl, —NR¹³C(O)NR¹⁴—C₁-C₆ alkyl, C₁-C₆ alkylthio, —CO₂R²¹, —S(O)R²², —SO₂R²³, —NR²⁴—C(═Z)—NR²⁵R²⁶ where Z is oxygen or N—CN, or a 3- to 10-membered saturated or unsaturated carbocyclic or heterocyclic ring system, the ring system itself being optionally substituted by one or more substituents independently selected from oxo, halogen, cyano, hydroxyl, C₁-C₆ alkyl, C₁-C₆ alkoxy, C₁-C₆ alkoxyC₁-C₆alkyl, trifluoromethyl and trifluoromethoxy), —C(O)NR¹⁵R¹⁶, or a 3- to 10-membered saturated or unsaturated carbocyclic or heterocyclic ring system optionally substituted by one or more substituents independently selected from oxo, halogen, cyano, hydroxyl, C₁-C₆ alkyl, C₁-C₆ alkoxy, C₁-C₆ alkoxyC₁-C₆alkyl, trifluoromethyl and trifluoromethoxy, or
R⁷ and R⁸ together with the nitrogen atom to which they are attached form a 3- to 8-membered saturated or partially saturated heterocyclic ring optionally containing one or more further ring heterogroups independently selected from nitrogen, S(O)ₘ and oxygen, the heterocyclic ring being optionally substituted by one or more substituents independently selected from oxo, hydroxyl, —C(O)NR¹⁷R¹⁸ and C₁-C₆ alkyl (optionally substituted by hydroxyl, C₁-C₆ alkoxy or —C(O)NR¹⁹R²⁰), with the proviso that the heterocyclic ring must be substituted unless
   (i) the heterocyclic ring is saturated and there is an SO or SO₂ ring heterogroup present, or
   (ii) the heterocyclic ring is partially saturated;
m is 0, 1 or 2;
Y represents an oxygen or sulphur atom or a group >NH;
R⁹ represents hydrogen, halogen, cyano, —S—CN, —C(O)N(R¹²)₂, C₁-C₆ alkoxycarbonyl, C₁-C₆ alkylcarbonyl (optionally substituted by —OC(O)CH₃), C₁-C₆ alkylcarbonyloxy, C₁-C₆ alkoxy, C₁-C₆ alkylthio, —C(O)—S—C₁-C₆ alkyl, —C(═CH₂)—O—CH₂OCH₃, C₁-C₆ alkyl, C₂-C₆ alkenyl, C₂-C₆ alkynyl or C₃-C₇ cycloalkyl, the latter four groups being optionally substituted by one or more substituents independently selected from halogen, hydroxyl, cyano, hydroxymethyl, C₁-C₄ alkoxy and C₁-C₄ alkylcarbonyloxy;
R¹⁰ represents C₁-C₆ alkyl (optionally substituted by halogen, C₁-C₄ alkoxy, C₁-C₄ alkylcarbonyloxy or C₃-C₇ cycloalkyl) or a 3- to 10-membered saturated or unsaturated carbocyclic or heterocyclic ring system which ring system may be optionally substituted by at least one substituent selected from halogen, carboxyl, hydroxyl, oxo, nitro, cyano, mercapto, C₁-C₆ alkyl, C₂-C₆ alkenyl, C₁-C₆ haloalkyl, C₁-C₆ hydroxyalkyl, C₁-C₆ alkoxy, C₁-C₆ haloalkoxy, C₁-C₆ alkylthio, C₁-C₆ alkylsulphinyl, C₁-C₆ alkylsulphonyl, C₁-C₆ alkylcarbonyl, C₁-C₆ alkylcarbonyloxy, C₁-C₆ alkoxycarbonyl, amino (—NH₂), carboxamido (—CONH₂), (mono) C₁-C₆ alkylamino, (di) C₁-C₆ alkylamino and phenyl;
R¹¹ represents a hydrogen atom or a methyl group;
each R¹² independently represents a hydrogen atom or a methyl group;
each of R¹³, R¹⁴, R¹⁵, R¹⁶, R¹⁷, R¹⁸, R¹⁹ and R²⁰ independently represents a hydrogen atom or a C₁-C₆ alkyl group;
each of R²¹, R²⁴, R²⁵ and R²⁶ independently represents a hydrogen atom or a C₁-C₆ alkyl or C₃-C₇ cycloalkyl group; and
each of R²² and R²³ independently represents a C₁-C₆ alkyl, C₃-C₇ cycloalkyl or a 5- to 6-membered saturated or unsaturated heterocyclic group;
   or a pharmaceutically acceptable salt thereof.

In the context of the present specification, unless otherwise stated, an alkyl, alkenyl or alkynyl substituent group or an alkyl, alkenyl or alkynyl moiety in a substituent group may be linear or branched. Examples of C₁-C₆ alkyl groups/moieties include methyl, ethyl, propyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, tert-butyl, n-pentyl, isopentyl, neopentyl and n-hexyl. Examples of C₂-C₆ alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1-hexadienyl. Examples of C₂-C₆ alkynyl groups/moieties include ethynyl, propynyl, 1-butynyl, 2-butynyl, 1-pentynyl and 1-hexynyl.

An alkylene, alkenylene or alkynylene linking group may be cyclic, linear or branched and may contain, for example, up to a total of eight carbon atoms. Examples of C₁-C₆ alkylene linking groups include methylene, ethylene, n-propylene, n-butylene, n-pentylene, n-hexylene, 1-methylethylene, 2-methylethylene, 1,2-dimethylethylene, 1-ethylethylene, 2-ethyl ethylene, 1-, 2- or 3-methylpropylene and 1-, 2- or 3-ethylpropylene; C₂-C₆ alkenylene linking groups containing one or more carbon-carbon double bonds include vinylidene, ethenylene (vinylene), propenylene, methyl ethenyl ene, 1-propenylidene, 2-propenylidene, 3-methylpropenylene, 3-ethylpropenylene, 1,3-dimethylpropenylene, 2,3-dimethylpropenylene, 3,3-dimethylpropenylene, 3-ethyl-1-methylpropenylene, 1,3,3-trimethylpropenylene and 2,3,3-trimethylpropenylene; and

C₂-C₆ alkynylene linking groups containing one or more carbon-carbon triple bonds include ethynylene, propynylene, and 2-butynylene.

A C₁-C₆ haloalkyl or C₁-C₆ haloalkoxy substituent group/moiety will comprise at least one halogen atom, e.g. one, two, three, four or five halogen atoms, examples of which include trifluoromethyl, trifluoromethoxy or pentafluoroethyl.

A C₁-C₆ hydroxyalkyl substituent group/moiety will comprise at least one hydroxyl group, e.g. one, two, three or four hydroxyl groups, examples of which include —CH₂OH, —CH₂CH₂OH, —CH₂CH₂CH₂OH, —CH(OH)CH₂OH, —CH(CH₃)OH and —CH(CH₂OH)₂. The alkyl groups in a di-C₁-C₆ alkylamino group/moiety may be the same as, or different from, one another.

In the definitions of R⁸ and R¹⁶, the saturated or unsaturated 3- to 10-membered carbocyclic or heterocyclic ring system may have alicyclic or aromatic properties. An unsaturated ring system will be partially or fully unsaturated. Similar comments apply in relation to the 5- to 6-membered saturated or unsaturated heterocyclic group in the definitions of R²² and R²³.

For the avoidance of doubt, it should be understood that the definitions of the heterocyclic groups/moieties in formula (I) are not intended to include unstable structures or any O—O, O—S or S—S bonds and that a substituent, if present, may be attached to any suitable ring atom.

When any chemical moiety or group in formula (I) is described as being optionally substituted, it will be appreciated that the moiety or group may be either unsubstituted or substituted by one or more of the specified substituents. It will be appreciated that the number and nature of substituents will be selected so as to avoid sterically undesirable combinations.

The following is a representation of formula (I) in which the ring carbon atoms have been numbered from 1 to 17:

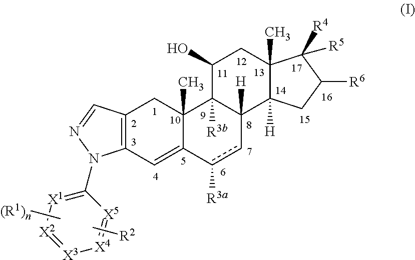

The dashed line between ring carbons 6 and 7 indicates an optional carbon-carbon bond. Thus there may be a single or double bond between ring carbons 6 and 7 in formula (I).

In one aspect, the invention provides compounds of formula (I) having the following structure:

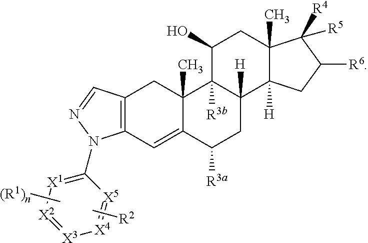

In formula (I), X¹, X², X³, X⁴ and X⁵ each represent CH (so as to form a phenyl ring) or, alternatively, one or two of X¹, X², X³, X⁴ and X⁵ may additionally represent a nitrogen atom (e.g. to form a pyridyl, pyrazinyl or pyridazinyl ring).

In an embodiment of the invention, X¹, X², X³, X⁴ and X⁵ each represent CH.

In another embodiment, one of X¹, X², X³, X⁴ and X⁵ represents a nitrogen atom and the others represent CH.

In a further embodiment, either X² and X³ each represent a nitrogen atom and X¹, X⁴ and X⁵ each represent CH, or, X³ and X⁴ each represent a nitrogen atom and X¹, X² and X⁵ each represent CH, or, X¹ and X⁴ each represent a nitrogen atom and X², X³ and X⁵ each represent CH, or, X² and X⁵ each represent a nitrogen atom and X¹, X³ and X⁴ each represent CH.

In an embodiment of the invention n is 0.

Thus, in one aspect, X¹, X², X³, X⁴ and X⁵ each represent CH and n is 0.

In another aspect, X¹, X², X³, X⁴ and X⁵ each independently represent CH or a nitrogen atom, provided that at least one and not more than two of X¹, X², X³, X⁴ and X⁵ simultaneously represent a nitrogen atom and n is 0 or 1.

In yet another aspect, X¹, X², X³, X⁴ and X⁵ each independently represent CH or a nitrogen atom, provided that only one of X¹, X², X³, X⁴ and X⁵ represents a nitrogen atom and n is 0.

R¹ represents a halogen atom (e.g. fluorine, chlorine, bromine or iodine) or a methyl or a methoxy group.

In an embodiment of the invention, R¹ represents a fluorine, chlorine or bromine atom, particularly a fluorine atom.

R² represents —C(O)NR⁷R⁸.

In one aspect, R² is attached to X² or X⁴ when X² or X⁴ is CH.

In one aspect of the invention,

R⁷ represents a hydrogen atom or a C₁-C₆, or C₁-C₄, or C₁-C₂ alkyl group, preferably a hydrogen atom or a methyl group, and
R⁸ represents
hydrogen,
C₁-C₆, or C₁-C₄, or C₁-C₂ alkyl [optionally substituted by one or more substituents, e.g. one, two, three or four substituents, independently selected from cyano, hydroxyl, C₁-C₆, or C₁-C₄, or C₁-C₂ alkoxy, C₁-C₆, or C₁-C₄, or C₁-C₂ haloalkoxy, —NR¹³R¹⁴, —C(O)NR¹³R¹⁴, —NR¹³C(O)C₁-C₆, or C₁-C₄, or C₁-C₂ alkyl, —NR¹³C(O)NR¹⁴—C₁-C₆, or C₁-C₄, or C₁-C₂ alkyl, C₁-C₆, or C₁-C₄, or C₁-C₂ alkylthio, —CO₂R²¹, —S(O)R²², —SO₂R²³, —NR²⁴—C(═Z)—NR²⁵R²⁶ where Z is oxygen or N—CN, or a 3- to 10-membered (e.g. 3-, 4-, 5- or 6- to 7-, 8-, 9- or 10-membered) saturated or unsaturated carbocyclic or heterocyclic ring system, the ring system itself being optionally substituted by one or more substituents, e.g. one, two, three or four substituents, independently selected from oxo, halogen (e.g. fluorine, chlorine, bromine or iodine), cyano, hydroxyl, C₁-C₆, or C₁-C₄, or C₁-C₂ alkyl, C₁-C₆, or C₁-C₄, or C₁-C₂ alkoxy, C₁-C₆ alkoxyC₁-C₆alkyl (such as methoxyC₁-C₆ alkyl or ethoxyC₁-C₆ alkyl), trifluoromethyl and trifluoromethoxy],
—C(O)NR¹⁵R¹⁶, or
a 3- to 10-membered (e.g. 3-, 4-, 5- or 6- to 7-, 8-, 9- or 10-membered) saturated or unsaturated carbocyclic or heterocyclic ring system optionally substituted by one or more substituents, e.g. one, two, three or four substituents, independently selected from oxo, halogen (e.g. fluorine, chlorine, bromine or iodine), cyano, hydroxyl, C₁-C₆, or C₁-C₄, or C₁-C₂ alkyl, C₁-C₆, or C₁-C₄, or C₁-C₂ alkoxy, C₁-C₆ alkoxyC₁-C₆alkyl (e.g. methoxyC₁-C₆ alkyl or ethoxyC₁-C₆ alkyl), trifluoromethyl and trifluoromethoxy.

The heterocyclic ring system will comprise at least one ring heteroatom (e.g. one, two, three or four ring heteroatoms independently) selected from nitrogen, sulphur and oxygen.

Examples of saturated or unsaturated 3- to 10-membered carbocyclic or heterocyclic ring systems that may be used, which may be monocyclic or polycyclic (e.g. bicyclic) in which the two or more rings are fused, include cyclopropyl, cyclobutyl, cyclopentyl, cyclohexyl, bicyclo[2.2.1]heptyl, cyclopentenyl, cyclohexenyl, phenyl, pyrrolidinyl, dioxidotetrahydrothiophenyl, piperidinyl, piperazinyl, morpholinyl, thiomorpholinyl, tetrahydrofuranyl, diazabicyclo[2.2.1]hept-2-yl, naphthyl, benzofuranyl, benzothienyl, benzodioxolyl, quinolinyl, oxazolyl, thiadiazolyl (e.g. 1,2,3-thiadiazolyl), 2,3-dihydrobenzofuranyl, tetrahydropyranyl, pyrazolyl, pyrazinyl, thiazolidinyl, indanyl, thienyl, isoxazolyl, pyridazinyl, pyrrolyl, furanyl, thiazolyl, indolyl, imidazolyl, pyrimidinyl, benzimidazolyl, triazolyl, tetrazolyl and pyridinyl.

Preferred ring systems include dioxidotetrahydrothiophenyl, cyclopentyl, pyridyl and tetrahydrofuranyl.

In one embodiment, R⁸ represents C₁-C₆, or C₁-C₄, or C₁-C₂ alkyl [optionally substituted by one or two substituents independently selected from C₁-C₆, or C₁-C₄, or C₁-C₂ alkoxy, —C(O)NR¹³R¹⁴, C₁-C₆, or C₁-C₄, or C₁-C₂ alkylthio, or a 3- to 10-membered saturated or unsaturated carbocyclic or heterocyclic ring system optionally substituted as hereinbefore defined] or a 3- to 10-membered saturated or unsaturated carbocyclic or heterocyclic ring system optionally substituted as hereinbefore defined.

In another embodiment, R⁸ represents C₁-C₂ alkyl optionally substituted by a methoxy, —CONH₂, —CONCH₃, methylthio or pyridyl group, or R⁸ represents dioxidotetrahydrothiophenyl, cyclopentyl or tetrahydrofuranyl.

In still another embodiment, R⁸ represents C₁-C₆, or C₁-C₄, or C₁-C₂ alkyl [optionally substituted by one or two substituents independently selected from C₁-C₆, or C₁-C₄, or C₁-C₂ alkoxy, —C(O)NR¹³R¹⁴ or C₁-C₆, or C₁-C₄, or C₁-C₂ alkylthio] or a 3- to 10-membered saturated or unsaturated carbocyclic or heterocyclic ring system optionally substituted as hereinbefore defined.

In yet another embodiment, R⁸ represents C₁-C₂ alkyl optionally substituted by a methoxy, —CONH₂ or methylthio group, or R⁸ represents dioxidotetrahydrothiophenyl.

Alternatively, R⁷ and R⁸ may together with the nitrogen atom to which they are attached form a 3- to 8-membered, preferably 5- to 6-membered, saturated or partially saturated heterocyclic ring optionally containing one or more (e.g. one or two) further ring heterogroups independently selected from nitrogen, S(O)ₘ and oxygen, the heterocyclic ring being optionally substituted by one or more substituents, e.g. one, two, three or four substituents, independently selected from oxo, hydroxyl, —C(O)NR¹⁷R¹⁸ and C₁-C₆, or C₁-C₄, or C₁-C₂ alkyl (optionally substituted by hydroxyl, C₁-C₆, or C₁-C₄, or C₁-C₂ alkoxy or —C(O)NR¹⁹R²⁰), with the proviso that the heterocyclic ring must be substituted unless

(i) the heterocyclic ring is saturated and there is an SO or SO₂ ring heterogroup present, or
(ii) the heterocyclic ring is partially saturated.

Thus, if one or more of conditions (i) to (ii) above apply, then the heterocyclic ring formed by R⁷ and R⁸ may be unsubstituted or substituted. If none of the conditions (i) to (ii) above applies, then the heterocyclic ring will be substituted.

Examples of 3- to 8-membered saturated or partially saturated heterocyclic rings include morpholinyl, azetidinyl, pyrrolidinyl, piperidinyl, piperazinyl, 3-pyrrolinyl, isoindolinyl, tetrahydroquinolinyl and thiomorpholinyl.

In one embodiment, R⁷ and R⁸ together with the nitrogen atom to which they are attached form a 5- to 6-membered saturated or partially saturated heterocyclic ring optionally containing one or two further ring heterogroups independently selected from nitrogen and oxygen, the heterocyclic ring being optionally substituted by one, two, three or four substituents independently selected from oxo, hydroxyl, —C(O)NR¹⁷R¹⁸ and C₁-C₆, or C₁-C₄, or C₁-C₂ alkyl (optionally substituted by hydroxyl, C₁-C₆, or C₁-C₄, or C₁-C₂ alkoxy or —C(O)NR¹⁹R²⁰), subject to the above proviso.

In another embodiment, R⁷ and R⁸ together with the nitrogen atom to which they are attached form a 5- to 6-membered saturated heterocyclic ring optionally containing one further ring heteroatom selected from nitrogen and oxygen (e.g. pyrrolidinyl or morpholinyl), the heterocyclic ring being optionally substituted by —C(O)NR¹⁷R¹⁸ (e.g. —CONH₂), subject to the above proviso.

In an embodiment of the invention R^{3a} represents a hydrogen atom or a methyl group and R^{3b} represents a hydrogen atom.

In another embodiment of the invention R^{3a} represents a hydrogen atom and R^{3b} represents a hydrogen atom.

In another embodiment of the invention R^{3a} represents a hydrogen atom and R^{3b} represents a fluorine atom.

R⁴ represents-C(O)—Y—CH(R¹¹)—R⁹ or —C(O)—CH(R¹¹)—Y—R⁹, preferably —C(O)—Y—CH(R¹¹)—R⁹.

R⁵ represents hydroxyl, —OCH₂SCH₃, —O—C(O)—R¹⁰, —O—C(O)—NH—R¹⁰, —O—C(O)—O—R¹⁰ or —O—C(O)—S—R¹⁰, in particular a hydroxyl or —O—C(O)—R¹⁰ group, and R⁶ represents a hydrogen or a halogen (e.g. fluorine, chlorine, bromine or iodine) atom or a hydroxyl or methyl group, particularly a hydrogen atom or methyl group.

In one embodiment, R⁵ represents a —O—C(O)—R¹⁰ group and R⁶ represents a hydrogen atom or a methyl group.

In another embodiment, R⁵ represents a —O—C(O)—R¹⁰ group and R⁶ represents a hydrogen atom.

Y represents an oxygen or sulphur atom or a group >NH, particularly an oxygen or sulphur atom.

R⁹ represents hydrogen, halogen (e.g. fluorine, chlorine, bromine or iodine), cyano, —S—CN, —C(O)N(R¹²)₂, C₁-C₆, or C₁-C₄, or C₁-C₂ alkoxycarbonyl, C₁-C₆, or C₁-C₄, or C₁-C₂ alkylcarbonyl (optionally substituted by —OC(O)CH₃), C₁-C₆, or C₁-C₄, or C₁-C₂ alkylcarbonyloxy, C₁-C₆, or C₁-C₄, or C₁-C₂ alkoxy, C₁-C₆, or C₁-C₄, or C₁-C₂ alkylthio, —C(O)—S—C₁-C₆, or C₁-C₄, or C₁-C₂ alkyl, —C(═CH₂)—O—CH₂OCH₃, C₁-C₆, or C₁-C₄, or C₁-C₂ alkyl, C₂-C₆ or C₂-C₄ alkenyl, C₂-C₆ or C₂-C₄ alkynyl or C₃-C₇, or C₅-C₆, cycloalkyl, the latter four groups being optionally substituted by one or more (e.g. one, two, three or four) substituents independently selected from halogen (e.g. fluorine, chlorine, bromine or iodine), hydroxyl, cyano, hydroxymethyl, C₁-C₄, or alkoxy and C₁-C₄, or C₁-C₂, alkylcarbonyloxy.

In an embodiment of the invention, R⁹ represents hydrogen, halogen (particularly fluorine), cyano, —S—CN, —C(O)N(R¹²)₂, C₁-C₂ alkoxycarbonyl, C₁-C₂ alkylcarbonyl (optionally substituted by —OC(O)CH₃), C₁-C₂ alkylcarbonyloxy, C₁-C₂ alkoxy, C₁-C₂ alkylthio, —C(O)—S—C₁-C₂ alkyl, —C(═CH₂)—O—CH₂OCH₃, C₁-C₆, or C₁-C₄, or C₁-C₂ alkyl, C₂-C₄ alkenyl, C₂-C₄ alkynyl or C₃-C₆ cycloalkyl, the latter four groups being optionally substituted by one or more (e.g. one, two, three or four) substituents independently selected from halogen (particularly fluorine or chlorine), hydroxyl, cyano, hydroxymethyl, C₁-C₄ alkoxy (particularly methoxy) and C₁-C₄ alkylcarbonyloxy (particularly methylcarbonyloxy).

In another embodiment of the invention, R⁹ represents hydrogen, halogen (particularly fluorine), cyano, methyl, hydroxymethyl or methylcarbonyl.

R¹⁰ represents C₁-C₆, or C₁-C₄, or C₁-C₂ alkyl (optionally substituted by at least one substituent, e.g. one, two, three or four substituents independently, selected from halogen (such as fluorine, chlorine, bromine or iodine), C₁-C₄, or C₁-C₂, alkoxy, C₁-C₄, or C₁-C₂, alkylcarbonyloxy and C₃-C₇, or C₅-C₆, cycloalkyl), or a 3- to 10-membered (e.g. 3-, 4-, 5- or 6- to 7-, 8-, 9- or 10-membered) saturated or unsaturated carbocyclic or heterocyclic ring system optionally substituted by at least one substituent (e.g. one, two, three or four substituents independently) selected from halogen (e.g. fluorine, chlorine, bromine or iodine), carboxyl, hydroxyl, oxo, nitro, cyano, mercapto, C₁-C₆, or C₁-C₄, or C₁-C₂ alkyl, C₂-C₆ or C₂-C₄ alkenyl, C₁-C₆, or C₁-C₄, or C₁-C₂ haloalkyl, C₁-C₆, or C₁-C₄, or C₁-C₂ hydroxyalkyl, C₁-C₆, or C₁-C₄, or C₁-C₂ alkoxy, C₁-C₆, or C₁-C₄, or C₁-C₂ haloalkoxy, C₁-C₆, or C₁-C₄, or C₁-C₂ alkylthio, C₁-C₆, or C₁-C₄, or C₁-C₂ alkylsulphinyl, C₁-C₆, or C₁-C₄, or C₁-C₂ alkylsulphonyl, C₁-C₆, or C₁-C₄, or C₁-C₂ alkylcarbonyl, C₁-C₆, or C₁-C₄, or C₁-C₂ alkylcarbonyloxy, C₁-C₆, or C₁-C₄, or C₁-C₂ alkoxycarbonyl, amino, carboxamido, (mono) C₁-C₆, or C₁-C₄, or C₁-C₂ alkylamino, (di) C₁-C₆, or C₁-C₄, or C₁-C₂ alkylamino and phenyl.

In one embodiment, R¹⁰ represents C₁-C₄, or C₁-C₃, or C₁-C₂ alkyl (optionally substituted by at least one substituent, e.g. one, two, three or four substituents independently, selected from halogen (particularly fluorine), C₁-C₂ alkoxy, C₁-C₂ alkylcarbonyloxy or C₅-C₆ cycloalkyl) or a 3- to 10-membered saturated or unsaturated carbocyclic or heterocyclic ring system optionally substituted as hereinbefore defined.

The heterocyclic ring system will comprise at least one ring heteroatom (e.g. one, two, three or four ring heteroatoms independently) selected from nitrogen, sulphur and oxygen.

Examples of saturated or unsaturated 3- to 10-membered carbocyclic or heterocyclic ring systems that may be used, which may be monocyclic or polycyclic (e.g. bicyclic) in which the two or more rings are fused, include one or more (in any combination) of cyclopropyl, cyclobutyl, cyclopentyl, cyclohexyl, bicyclo[2.2.1]heptyl, cyclopentenyl, cyclohexenyl, phenyl, pyrrolidinyl, piperidinyl, piperazinyl, morpholinyl, thiomorpholinyl, tetrahydrofuranyl, diazabicyclo[2.2.1]hept-2-yl, naphthyl, benzofuranyl, benzothicnyl, benzodioxolyl, quinolinyl, oxazolyl, thiadiazolyl (e.g. 1,2,3-thiadiazolyl), 2,3-dihydrobenzofuranyl, tetrahydropyranyl, pyrazolyl, pyrazinyl, thiazolidinyl, indanyl, thienyl, isoxazolyl, pyridazinyl, pyrrolyl, furanyl, thiazolyl, indolyl, indazolyl, imidazolyl, pyrimidinyl, benzimidazolyl, triazolyl, tetrazolyl and pyridinyl. Preferred ring systems include thiadiazolyl, furanyl, thiazolyl, cyclopropyl, cyclobutyl, imidazolyl, oxazolyl, triazolyl, isoxazolyl, thienyl, tetrahydrofuranyl, indazolyl, tetrahydropyranyl and pyrrolyl.

Preferred substituents on the 3- to 10-membered saturated or unsaturated carbocyclic or heterocyclic ring system include alkyl, alkoxy and cyano substituent groups.

In an embodiment of the invention, R¹⁰ represents a 3-, 4- or 5- to 6-, 7- or 8-membered saturated or unsaturated carbocyclic or heterocyclic ring system optionally substituted by one, two, three or four substituents independently selected from halogen, carboxyl, hydroxyl, oxo, nitro, cyano, mercapto, C₁-C₆, or C₁-C₄, or C₁-C₂ alkyl, C₂-C₆ or C₂-C₄ alkenyl, C₁-C₆, or C₁-C₄, or C₁-C₂ haloalkyl, C₁-C₆, or C₁-C₄, or C₁-C₂ hydroxyalkyl, C₁-C₆, or C₁-C₄, or C₁-C₂ alkoxy, C₁-C₆, or C₁-C₄, or C₁-C₂ haloalkoxy, C₁-C₆, or C₁-C₄, or C₁-C₂ alkylthio, C₁-C₆, or C₁-C₄, or C₁-C₂ alkylsulphinyl, C₁-C₆, or C₁-C₄, or C₁-C₂ alkylsulphonyl, C₁-C₆, or C₁-C₄, or C₁-C₂ alkylcarbonyl, C₁-C₆, or C₁-C₄, or C₁-C₂ alkylcarbonyloxy, C₁-C₆, or C₁-C₄, or C₁-C₂ alkoxycarbonyl, amino, carboxamido, (mono) C₁-C₆, or C₁-C₄, or C₁-C₂ alkylamino, (di) C₁-C₆, or C₁-C₄, or C₁-C₂ alkylamino and phenyl.

In another embodiment, R¹⁰ represents a 3- to 6-membered saturated or unsaturated carbocyclic or heterocyclic ring system such as a thiadiazolyl, furanyl, thiazolyl, indazolyl, cyclopropyl, cyclobutyl, imidazolyl, oxazolyl, triazolyl, isoxazolyl, thienyl, tetrahydrofuranyl, tetrahydropyranyl or pyrrolyl ring, the ring system being optionally substituted by at least one substituent (e.g. one, two, three or four, preferably one or two, substituents independently) selected from cyano, C₁-C₄ alkyl (particularly methyl) and C₁-C₄ alkoxy (particularly methoxy).

In still another embodiment, R¹⁰ represents either C₁-C₄, or C₁-C₃, or C₁-C₂ alkyl optionally substituted by C₁-C₂ alkoxy (e.g. methoxymethyl), or a cyclopropyl, oxazolyl, indazolyl, tetrahydrofuranyl or furanyl ring.

In a further embodiment, R¹⁰ represents either C₁-C₄, or C₁-C₃, or C₁-C₂ alkyl optionally substituted by C₁-C₂ alkoxy (e.g. methoxymethyl), or a cyclopropyl, oxazolyl or furanyl ring.

In an embodiment of the invention, R¹¹ represents a hydrogen atom.

Examples of compounds of the invention include:
(1R,3aS,3bS,10aR,10bS,11S,12aS)-1-{[(Cyanomethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aR,10bS,11S,12aS)-1-{[(Fluoromethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aR,10bS,11S,12aS)-1-{[(Fluoromethyl)sulfanyl]carbonyl}-11-hydroxy-7-{3-[(2-methoxyethyl)carbamoyl]phenyl}-10a,12a-dimethyl-1,2,3,3a,3 b,4,5,7,10,10a,10b,11,12,12a-tetradecahydrocyclopenta[5,6]naphtho[1,2-f]indazol-1-ylmethoxyacetate,
(1R,3 aS,3bS,10aR,10bS,11S,12aS)-1-{[(Cyanomethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a, 10b,11,12,12a-tetradecahydrocyclopenta[5,6]naphtho[1,2-f]indazol-1-yl cyclopropanecarboxylate,
(1R,3 aS,3bS,10aR,10bS,11S,12aS)-1-{[(Fluoromethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a, 10b,11,12,12a-tetradecahydrocyclopenta[5,6]naphtho[1,2-f]indazol-1-yl cyclopropanecarboxylate,
(1R,3 aS,3bS,10aR,10bS,11S,12aS)-1-{[(Cyanomethyl)sulfanyl]carbonyl}-11-hydroxy-10a,12a-dimethyl-7-(3-{[2-(methylsulfanyl)ethyl]carbamoyl}phenyl)-1,2,3,3a,3 b,4,5,7,10,10a, 10b,11,12,12a-tetradecahydrocyclopenta[5,6]naphtho[1,2-t]indazol-1-yl propanoate,
(1R,3aS,3bS,10aR,10bS,11S,12aS)-1-{[(Fluoromethyl)sulfanyl]carbonyl}-11-hydroxy-10a,12a-dimethyl-7-(3-{[2-(methylsulfanyl)ethyl]carbamoyl}phenyl)-1,2,3,3a,3b,4,5,7,10,10a,10b,11,12,12a-tetradecahydrocyclopenta[5,6]naphtho[1,2-f]indazol-1-yl propanoate,
(1R,3 aS,3bS,10aR,10bS,11S,12aS)-1-{[(Fluoromethyl)sulfanyl]carbonyl}-11-hydroxy-10a,12a-dimethyl-7-(3-{[2-(methylsulfanyl)ethyl]carbamoyl}phenyl)-1,2,3,3a,3b,4,5,7,10,10a,10b,11,12,12a-tetradecahydrocyclopenta[5,6]naphtho[1,2-f]indazol-1-yl methoxyacetate,
(1R,3 aS,3bS,10aR,10bS,11S,12aS)-1-{[(Cyanomethyl)sulfanyl]carbonyl}-11-hydroxy-10a,12a-dimethyl-7-(3-{[2-(methylsulfanyl)ethyl]carbamoyl}phenyl)-1,2,3,3a,3b,4,5,7,10,10a, 10b,11,12,12a-tetradecahydrocyclopenta[5,6]naphtho[1,2-f]indazol-1-yl cyclopropanecarboxylate,
(1R,3 aS,3bS,10aR,10bS,11S,12aS)-1-{[(Fluoromethyl)sulfanyl]carbonyl}-11-hydroxy-10a,12a-dimethyl-7-(3-{[2-(methylsulfanyl)ethyl]carbamoyl}phenyl)-1,2,3,3a,3b,4,5,7,10,10a, 10b,11,12,12a-tetradecahydrocyclopenta[5,6]naphtho[1,2-f]indazol-1-yl cyclopropanecarboxylate,
(1R,3 aS,3bS,1 OaR,10bS,11S,12aS)-7-{3-[(2-Amino-2-oxoethyl)carbamoyl]phenyl}-1-{[(cyanomethyl)sul fanyl]carbonyl}-11-hydroxy-10a,12a-dimethyl-1,2,3,3a,3b,4,5,7,10,10a, 10b,11,12,12a-tetradecahydrocyclopenta[5,6]naphtho[1,2-f]indazol-1-yl propanoate,
(1R,3 aS,3bS,10aR,10bS,11S,12aS)-1-{[(Cyanomethyl)sulfanyl]carbonyl}-7-{3-[(1,1-dioxidotetrahydrothiophen-3-yl)carbamoyl]phenyl}-11-hydroxy-10a,12a-dimethyl-1,2,3,3a,3b,4,5,7,10,10a,10b,11,12,12a-tetradecahydrocyclopenta[5,6]naphtho[1,2-n]indazol-1-yl propanoate,
(1R,3 aS,3bS,10aR,10bS,11S,12aS)-7-(3-{[(2R)-2-Carbamoylpyrrolidin-1-yl]carbonyl}phenyl)-1-{[(fluoromethyl)sulfanyl]carbonyl}-11-hydroxy-10a,12a-dimethyl-1,2,3,3a,3b,4,5,7,10,10a, 10b,11,12,12a-tetradecahydrocyclopenta[5,6]naphtho[1,2-f]indazol-1-yl propanoate,
(1R,3 aS,3bS,10aR,10bS,11S,12aS)-7-(3-{[(2R)-2-Carbamoylpyrrolidin-1-yl]carbonyl}phenyl)-1-{[(cyanomethyl)sulfanyl]carbonyl}-11-hydroxy-10a,12a-dimethyl-1,2,3,3a,3 b,4,5,7,10,10a,10b,11,12,12a-tetradecahydrocyclopenta[5,6]naphtho[1,2-f]indazol-1-yl propanoate, and
(1R,3aS,3bS,10aS,10bR,11S,12aS)-7-(3-{[(2R)-2-Carbamoylpyrrolidin-1-yl]carbonyl}phenyl)-10b-fluoro-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl methoxyacetate,
(1R,3aS,3bS,10aR,10bS,11S,12aS)-7-(3-{[(2R)-2-Carbamoylpyrrolidin-1-yl]carbonyl}phenyl)-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl methoxyacetate,
(1R,3 aS,3bS,10aS,10bR,11S,12aS)-7-{3-[(2-Amino-2-oxoethyl)carbamoyl]phenyl}-10b-fluoro-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl methoxyacetate,
(1R,3 aS,3bS,10aR,10bS,11S,12aS)-7-{3-[(2-Amino-2-oxoethyl)carbamoyl]phenyl}-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,2R,3aS,3bS,10aS,10bR,11S,12aS)-7-(3-{[(1S)-2-Amino-1-methyl-2-oxoethyl]carbamoyl}phenyl)-10b-fluoro-1-{[(fluoromethyl)sulfanyl]carbonyl}-11-hydroxy-2,10a,12a-trimethyl-1,2,3,3a,3b,4,5,7,10,10a,10b,11,12,12a-tetradecahydrocyclopenta[5,6]naphtho[1,2-f]indazol-1-yl methoxyacetate,
(1R,3aS,3bS,5S,10aR,10bS,11S,12aS)-7-(3-{[(1S)-2-Amino-1-methyl-2-oxoethyl]carbamoyl}phenyl)-1-{[(fluoromethyl)sulfanyl]carbonyl}-11-hydroxy-5,10a,12a-trimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,5S,10aR,10bS,11S,12aS)-7-(3-{[(1R)-2-Amino-1-methyl-2-oxoethyl]carbamoyl}phenyl)-1-{[(fluoromethyl)sulfanyl]carbonyl}-11-hydroxy-5,10a,12a-trimethyl-1,2,3,3a,3b,4,5,7,10,10a,10b,11,12,12a-tetradecahydrocyclopenta[5,6]naphtho[1,2-f]indazol-1-yl propanoate,
(1R,2R,3aS,3bS,10aS,10bR,11S,12aS)-7-(3-{[(1S)-2-Amino-1-methyl-2-oxoethyl]carbamoyl}phenyl)-10b-fluoro-1-{[(fluoromethyl)sulfanyl]carbonyl}-11-hydroxy-2,10a,12a-trimethyl-1,2,3,3a,3b,4,5,7,10,10a,10b,11,12,12a-tetradecahydrocyclopenta[5,6]naphtho[1,2-f]indazol-1-yl propanoate,
(1R,2R,3aS,3bS,10aS,10bR,11S,12aS)-7-(3-{[(1R)-2-Amino-1-methyl-2-oxoethyl]carbamoyl}phenyl)-10b-fluoro-1-{[(fluoromethyl)sulfanyl]carbonyl}-11-hydroxy-2,10a,12a-trimethyl-1,2,3,3a,3b,4,5,7,10,10a,10b,11,12,12a-tetradecahydrocyclopenta[5,6]naphtho[1,2-f]indazol-1-yl propanoate,
(1R,2R,3aS,3bS,10aS,10bR,11S,12aS)-7-[3-(Ethylcarbamoyl)phenyl]-10b-fluoro-1-{[(fluoromethyl)sulfanyl]carbonyl}-11-hydroxy-2,10a,12a-trimethyl-1,2,3,3a,3b,4,5,7,10,10a,10b,11,12,12a-tetradecahydrocyclopenta[5,6]naphtho[1,2-f]indazol-1-yl methoxyacetate,
(1R,2R,3aS,3bS,10aS,10bR,11S,12aS)-10b-Fluoro-1-{[(fluoromethyl)sulfanyl]carbonyl}-11-hydroxy-2,10a,12a-trimethyl-7-[3-(methylcarbamoyl)phenyl]-1,2,3,3a,3b,4,5,7,10,10a,10b,11,12,12a-tetradecahydrocyclopenta[5,6]naphtho[1,2-f]indazol-1-yl methoxyacetate,
(1R,2R,3aS,3bS,10aS,10bR,11S,12aS)-7-(3-{[(1R)-2-Amino-1-methyl-2-oxoethyl]carbamoyl}phenyl)-10b-fluoro-1-{[(fluoromethyl)sulfanyl]carbonyl}-11-hydroxy-2,10a,12a-trimethyl-1,2,3,3a,3b,4,5,7,10,10a,10b,11,12,12a-tetradecahydrocyclopenta[5,6]naphtho[1,2-f]indazol-1-yl methoxyacetate,
(1R,3aS,3bS,5S,10aR,10bS,11S,12aS)-7-{3-[(2-Amino-2-oxoethyl)carbamoyl]phenyl}-1-{[(fluoromethyl)sulfanyl]carbonyl}-11-hydroxy-5,10a,12a-trimethyl-1,2,3,3a,3b,4,5,7,10,10a,10b,11,12,12a-tetradecahydrocyclopenta[5,6]naphtho[1,2-f]indazol-1-yl (2R)-tetrahydrofuran-2-carboxylate,
(1R,3aS,3bS,5S,10aR,10bS,11S,12aS)-7-{3-[(2-Amino-2-oxoethyl)carbamoyl]phenyl}-1-{[(fluoromethyl)sul fanyl]carbonyl}-11-hydroxy-5,10a,12a-trimethyl-1,2,3,3a,3b,4,5,7,10,10a,10b,11,12,12a-tetradecahydrocyclopenta[5,6]naphtho[1,2-f]indazol-1-yl cyclopropanecarboxylate,
(1R,3aS,3bS,10aS,10bR,11S,12aS)-7-(3-{[(1R)-2-Amino-1-methyl-2-oxoethyl]carbamoyl}phenyl)-10b-fluoro-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aS,10bR,11S,12aS)-10b-Fluoro-1-{[(fluoromethyl)sulfanyl]carbonyl}-11-hydroxy-10a,12a-dimethyl-7-[3-(methylcarbamoyl)phenyl]-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aS,10bR,11S,12aS)-7-{3-[(2-Amino-2-oxoethyl)(methyl)carbamoyl]phenyl}-10b-fluoro-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aS,10bR,11S,12aS)-7-(3-{[(1S)-2-Amino-1-methyl-2-oxoethyl]carbamoyl}phenyl)-10b-fluoro-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aS,10bR,11S,12aS)-10b-Fluoro-1-{[(fluoromethyl)sulfanyl]carbonyl}-11-hydroxy-10a,12a-dimethyl-7-(3-{[2-(methylamino)-2-oxoethyl]carbamoyl}phenyl)-1,2,3,3a,3b,4,5,7,10,10a,10b,11,12,12a-tetradecahydrocyclopenta[5,6]naphtho[1,2-f]indazol-1-yl propanoate,
(1R,3aS,3bS,5S,10aR,10bS,11S,12aS)-7-{3-[(2-Amino-2-oxoethyl)carbamoyl]phenyl}-1-{[(fluoromethyl)sulfanyl]carbonyl}-11-hydroxy-5,10a,12a-trimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,5S,10aR,10bS,11S,12aS)-7-{3-[(2-Amino-2-oxoethyl)carbamoyl]phenyl}-1-{[(fluoromethyl)sulfanyl]carbonyl}-11-hydroxy-5,10a,12a-trimethyl-1,2,3,3a,3b,4,5,7,10,10a,10b,11,12,12a-tetradecahydrocyclopenta[5,6]naphtho[1,2-f]indazol-1-yl methoxyacetate,
(1R,3aS,3bS,10aR,10bS,11S,12aS)-1-{[(Fluoromethyl)sulfanyl]carbonyl}-11-hydroxy-10a,12a-dimethyl-7-{3-[(pyridin-3-ylmethyl)carbamoyl]pheny}-1,2,3,3a,3b,4,5,7,10,10a,10b,11,12,12a-tetradecahydrocyclopenta[5,6]naphtho[1,2-f]indazol-1-yl 1,3-oxazole-4-carboxylate,
(1R,3aS,3bS,10aR,10bS,11S,12aS)-1-{[(Fluoromethyl)sulfanyl]carbonyl}-11-hydroxy-10a,12a-dimethyl-7-{3-[(3R)-tetrahydrofuran-3-ylcarbamoyl]phenyl}-1,2,3,3a,3b,4,5,7,10,10a,10b,11,12,12a-tetradecahydrocyclopenta[5,6]naphtho[1,2-f]indazol-1-yl methoxyacetate,
(1R,2R,3aS,3bS,10aS,10bR,11S,12aS)-7-{3-[(2-Amino-2-oxoethyl)carbamoyl]phenyl}-10b-fluoro-1-{[(fluoromethyl)sulfanyl]carbonyl}-11-hydroxy-2,10a,12a-trimethyl-1,2,3,3a,3b,4,5,7,10,10a,10b,11,12,12a-tetradecahydrocyclopenta[5,6]naphtho[1,2-f]indazol-1-yl propanoate,
(1R,2R,3aS,3bS,10aS,10bR,11S,12aS)-7-(3-{[(2R)-2-Carbamoylpyrrolidin-1-yl]carbonyl}phenyl)-10b-fluoro-1-{[(fluoromethyl)sulfanyl]carbonyl}-11-hydroxy-2,10a,12a-trimethyl-1,2,3,3a,3b,4,5,7,10,10a,10b,11,12,12a-tetradecahydrocyclopenta[5,6]naphtho[1,2-f]indazol-1-yl propanoate,
(1R,2R,3aS,3bS,10aS,10bR,11S,12aS)-10b-Fluoro-1-{[(fluoromethyl)sulfanyl]carbonyl}-11-hydroxy-2,10a,12a-trimethyl-7-{3-[(pyridin-3-ylmethyl)carbamoyl]phenyl}-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aS,10bR,11S,12aS)-7-{3-[(2-Amino-2-oxoethyl)carbamoyl]phenyl}-10b-fluoro-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,2R,3aS,3bS,10aS,10bR,11S,12aS)-7-{3-[(2-Amino-2-oxoethyl)carbamoyl]phenyl}-10b-fluoro-1-{[(fluoromethyl)sulfanyl]carbonyl}-11-hydroxy-2,10a,12a-trimethyl-1,2,3,3a,3b,4,5,7,10,10a,10b,11,12,12a-tetradecahydrocyclopenta[5,6]naphtho[1,2-f]indazol-1-yl methoxyacetate,
(1R,2R,3aS,3bS,10aS,10bR,11S,12aS)-10b-Fluoro-1-{[(fluoromethyl)sulfanyl]carbonyl}-11-hydroxy-2,10a,12a-trimethyl-7-{3-[(pyridin-3-ylmethyl)carbamoyl]phenyl}-1,2,3,3a,3b,4,5,7,10,10a,10b,11,12,12a-tetradecahydrocyclopenta[5,6]naphtho[1,2-f]indazol-1-yl methoxyacetate,
(1R,2R,3aS,3bS,10aS,10bR,11S,12aS)-7-(3-{[(2R)-2-Carbamoylpyrrolidin-1-yl]carbonyl}phenyl)-10b-fluoro-1-{[(fluoromethyl)sulfanyl]carbonyl}-11-hydroxy-2,10a,12a-trimethyl-1,2,3,3a,3b,4,5,7,10,10a,10b,11,12,12a-tetradecahydrocyclopenta[5,6]naphtho[1,2-f]indazol-1-yl methoxyacetate,
(1R,3aS,3bS,10aR,10bS,11S,12aS)-7-(3-{[(2R)-2-Carbamoylpyrrolidin-1-yl]carbonyl}phenyl)-11-hydroxy-1-{[(2-hydroxyethyl)sulfanyl]carbonyl}-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aR,10bS,11S,12aS)-7-(3-{[(2R)-2-Carbamoylpyrrolidin-1-yl]carbonyl}phenyl)-1-{[(cyanomethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl 1,3-oxazole-4-carboxylate,
(1R,3aS,3bS,10aR,10bS,11S,12aS)-7-(3-{[(2R)-2-Carbamoylpyrrolidin-1-yl]carbonyl}phenyl)-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl 1,3-oxazole-4-carboxylate,
(1R,3aS,3bS,10aR,10bS,11S,12aS)-7-{3-[(2-Amino-2-oxoethyl)carbamoyl]phenyl}-1-{[(cyanomethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl methoxyacetate,
(1R,3aS,3bS,10aR,10bS,11S,12aS)-1-{[(Cyanomethyl)sulfanyl]carbonyl}-11-hydroxy-10a,12a-dimethyl-7-{3-[(pyridin-3-ylmethyl)carbamoyl]phenyl}-1,2,3,3a,3b,4,5,7,10,10a,10b,11,12,12a-tetradecahydrocyclopenta[5,6]naphtho[1,2-f]indazol-1-yl methoxyacetate,
(1R,3aS,3bS,10aS,10bR,11S,12aS)-7-(3-{[(2R)-2-Carbamoylpyrrolidin-1-yl]carbonyl}phenyl)-10b-fluoro-11-hydroxy-10a,12a-dimethyl-1-[(methylsulfanyl)carbonyl]-1,2,3,3a,3b,4,5,7,10,10a,10b,11,12,12a-tetradecahydrocyclopenta[5,6]naphtho[1,2-f]indazol-1-yl cyclopropanecarboxylate,
(1R,3aS,3bS,10aS,10bR,11S,12aS)-7-(3-{[(2R)-2-Carbamoylpyrrolidin-1-yl]carbonyl}phenyl)-1-{[(cyanomethyl)sulfanyl]carbonyl}-10b-fluoro-11-hydroxy-10a,12a-dimethyl-1,2,3,3a,3b,4,5,7,10,10a,10b,11,12,12a-tetradecahydrocyclo-pcnta[5,6]naphtho[1,2-f]indazol-1-yl cyclopropanecarboxylate,
(1R,3aS,3bS,10aS,10bR,11S,12aS)-7-(3-{[(2R)-2-Carbamoylpyrrolidin-1-yl]carbonyl}phenyl)-10b-fluoro-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl cyclopropanecarboxylate,
(1R,3aS,3bS,10aS,10bR,11S,12aS)-7-(3-{[(2R)-2-Carbamoylpyrrolidin-1-yl]carbonyl}phenyl)-10b-fluoro-11-hydroxy-10a,12a-dimethyl-1-[(methylsulfanyl)carbonyl]-1,2,3,3a,3b,4,5,7,10,10a,10b,11,12,12a-tetradecahydrocyclopenta[5,6]naphtho[1,2-f]indazol-1-yl methoxyacetate,
(1R,3aS,3bS,10aS,10bR,11S,12aS)-7-(3-{[(2R)-2-Carbamoylpyrrolidin-1-yl]carbonyl}phenyl)-1-{[(cyanomethyl)sulfanyl]carbonyl}-10b-fluoro-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl methoxyacetate,
(1R,3aS,3bS,10aS,10bR,11S,12aS)-7-(3-{[(2R)-2-Carbamoylpyrrolidin-1-yl]carbonyl}phenyl)-10b-fluoro-11-hydroxy-1-{[(2-hydroxyethyl)sulfanyl]carbonyl}-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aS,10bR,11S,12aS)-7-(3-{[(2R)-2-Carbamoylpyrrolidin-1-yl]carbonyl}phenyl)-10b-fluoro-11-hydroxy-10a,12a-dimethyl-1-[(methylsulfanyl)carbonyl]-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aS,10bR,11S,12aS)-7-(3-{[(2R)-2-Carbamoylpyrrolidin-1-yl]carbonyl}phenyl)-1-{[(cyanomethyl)sulfanyl]carbonyl}-10b-fluoro-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aS,10bR,11S,12aS)-7-(3-{[(2R)-2-Carbamoylpyrrolidin-1-yl]carbonyl}phenyl)-10b-fluoro-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3 aS,3bS,10aR,10bS,11S,12aS)-7-[3-(Cyclopentylcarbamoyl)phenyl]-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate,
   and pharmaceutically acceptable salts of any one thereof.

It should be noted that each of the chemical compounds listed above represents a particular and independent aspect of the invention.

The present invention further provides a process for the preparation of a compound of formula (I) or a pharmaceutically acceptable salt thereof as defined above which comprises

(i) reacting a compound of formula (II)

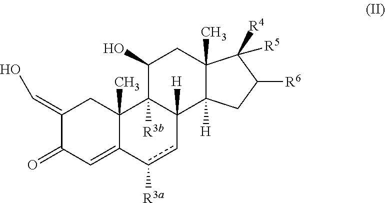

wherein R^{3a}, R^{3b}, R⁴, R⁵ and R⁶ are as defined in formula (I), with a compound of formula (III) or an acid addition salt (e.g. hydrochloride salt) thereof

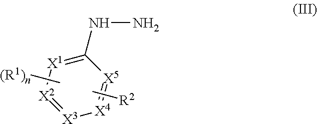

wherein n, R¹, R², X¹, X², X³, X⁴ and X⁵ are as defined in formula (I); or
(ii) when R⁴ represents —C(O)—Y—CH(R¹¹)—R⁹ and Y represents a sulphur atom, reacting a compound of formula (IV)

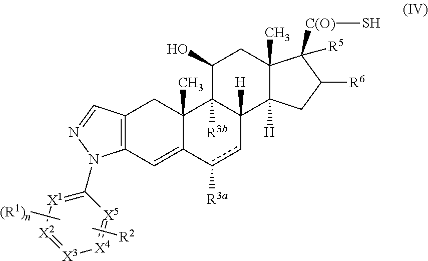

where n, X¹, X², X³, X⁴, X⁵, R¹, R², R^{3a}, R^{3b}, R⁵ and R⁶ defined are as defined in formula (I), with a compound of formula (V), R⁹—CH(R¹¹)-L, where L represents a leaving group (e.g. a halogen atom) and R⁹ and R¹¹ are as defined in formula (I); or
(iii) reacting a compound of formula (VI)

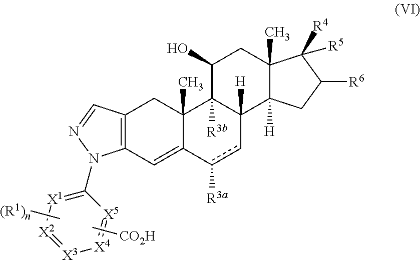

where n, X¹, X², X³, X⁴, X⁵, R¹, R^{3a}, R^{3b}, R⁴, R⁵ and R⁶ are as defined in formula (I), with a compound of formula (VII), HNR⁷R⁸, wherein R⁷ and R⁸ are as defined in formula (I); and optionally thereafter carrying out one or more of the following procedures:
converting a compound of formula (I) into another compound of formula (I)
removing any protecting groups
forming a pharmaceutically acceptable salt.

Process (i) above is conveniently carried out in the presence of an organic solvent such as acetic acid/water mixture at room temperature (20° C.) or, alternatively, in the presence of an organic solvent such as ethanol at a temperature in the range from room temperature (20° C.) to 90° C. Preferably, the reaction is carried out in the presence of a base, e.g. an alkali metal acetate such as potassium acetate.

The process (ii) above is conveniently carried out in the presence of an organic solvent such as dichloromethane, N,N-dimethylformamide or acetone in the presence of a base (e.g. Hünig's base or an alkali metal base such potassium carbonate, sodium carbonate or sodium hydrogen carbonate) at a temperature in the range from, for example, 25° C. to 35° C.

Process (iii) above is conveniently carried out in the presence of an organic solvent such as N,N-diisopropylethylamine, for example, at room temperature. Advantageously, a coupling agent may be used, e.g. 2-(1H-benzo[d][1,2,3]triazol-1-yl)-1,1,3,3-tetramethylisouronium tetrafluoroborate.

The compounds of formula (II) may be prepared by reacting a compound of formula (X)

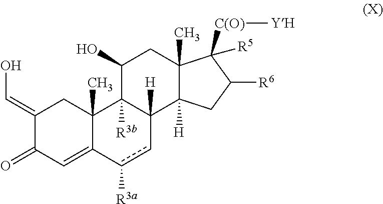

wherein Y′ represents an oxygen or sulphur atom and R^{3a}, R^{3b}, R⁵ and R⁶ are as defined in formula (II), with a compound of formula (V) above optionally followed by reaction with an amine of formula (XI), R⁹—CH(R¹¹)—NH₂, to obtain compounds of formula (II) in which R⁴ is —C(O)—Y—CH(R¹¹)—R⁹ where Y is NH, or with R⁹—Y—CH(R¹¹)-L¹ (formula XIA), wherein L¹ is a leaving group (e.g. a halogen atom) and R⁹ and R¹¹ are as defined in formula (I).

Compounds of formula (X) in which R⁵ is other than hydroxyl may be prepared by reacting a compound of formula (XII)

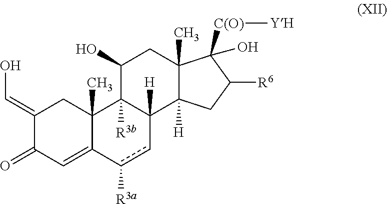

wherein R^{3a}, R^{3b}, R⁶ and Y′ are as defined in formula (X), with L²-CH₂SCH₃ (formula XV), L²-C(O)—R¹⁰ (formula XVI), L²-C(O)—NH—R¹⁰ (formula XVII), L²-C(O)—O—R¹⁰ (formula XVIII) or L²-C(O)—S—R¹⁰ (formula XIX) where L² represents a leaving group and R¹⁰ is as defined in formula (I).

Compounds of formula (XII) (being a compound of formula (X) in which R⁵ is hydroxyl) to wherein Y′ is sulphur may be prepared by reacting a corresponding compound of formula (XII) wherein Y′ is oxygen with hydrogen sulphide according to methods known in the art.

Compounds of formula (XII) wherein Y′ is oxygen may be prepared by reacting a compound of formula (XIII)

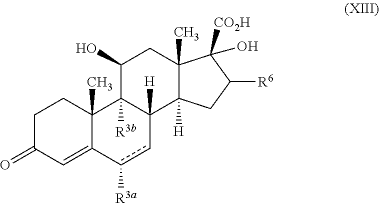

wherein R^{3a}, R^{3b} and R⁶ are as defined in formula (XII), with methyl or ethyl formate in the presence of a base such as sodium hydride, in a manner analogous to the method described in the journal article by Wuest, F. et al., Steroids, 68 (2003), 177-191.

Compounds of formula (XIII) containing a carbon-carbon double bond in the 6,7 position may be prepared from compounds of formula (XIV)

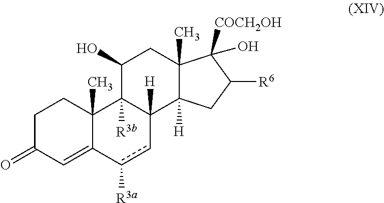

wherein R^{3a}, R^{3b} and R⁶ are as defined in formula (XIII), by introducing a suitable protecting group on the —C(O)CH₂OH group, followed by a dehydrogenation reaction to form a carbon-carbon double bond in the 6,7 position, then followed by removal of the protecting group and lastly by an oxidative degradation reaction, all such reaction steps being carried out according to processes known in the art.

Compounds of formula (IV) may be prepared by reacting a compound of formula (X) as defined above in which Y′ is oxygen with a compound of formula (III) as defined above, followed by reaction with hydrogen sulphide to convert Y′ from oxygen to sulphur according to methods known in the art.

Alternatively, compounds of formula (IV) may be prepared by reacting a compound of formula (XII) in which Y′ is oxygen with a compound of formula (III) as defined above, followed by reaction with hydrogen sulphide to convert Y′ from oxygen to sulphur, optionally followed by reaction with a compound of formula (XV) to (XIX).

Compounds of formula (VI) may be prepared by processes analogous to steps (i) and (ii) above.

Compounds of formulae (III), (V), (VII), (XI), (XIA), (XIV), (XV), (XVI), (XVII), (XVIII) and (XIX) are either commercially available, are well known in the literature or may be prepared easily using known techniques.

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J.W.F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) above may be converted to a pharmaceutically acceptable salt thereof, preferably an acid addition salt such as a hydrochloride, hydrobromide, trifluoroacetate, sulfate, phosphate, acetate, fumarate, maleate, tartrate, lactate, citrate, pyruvate, succinate, oxalate, methanesulphonate or p-toluenesulphonate.

The compounds of formula (I) and pharmaceutically acceptable salts thereof may exist in solvated, for example hydrated, as well as unsolvated forms, and the present invention encompasses all such solvated forms.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically and diastereomerically pure forms are particularly desired.

The compounds of formula (I) and their pharmaceutically acceptable salts have activity as pharmaceuticals, in particular as modulators of glucocorticoid receptor activity, and thus may be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogcnic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia arcata, male-pattern baldness, Sweet's syndrome, Weber-Christian syndrome, erythema multiforme; cellulitis, both infective and non-infective; panniculitis;cutaneous lymphomas, non-melanoma skin cancer and other dysplastic lesions; drug-induced disorders including fixed drug eruptions;
3. eyes: blepharitis; conjunctivitis, including perennial and vernal allergic conjunctivitis; iritis; anterior and posterior uveitis; choroiditis; autoimmune, degenerative or inflammatory disorders affecting the retina; ophthalmitis including sympathetic ophthalmitis; sarcoidosis; infections including viral, fungal, and bacterial;
4. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
5. allograft rejection: acute and chronic following, for example, transplantation of kidney, heart, liver, lung, bone marrow, skin or cornea or following blood transfusion; or chronic graft versus host disease;
6. other auto-immune and allergic disorders including rheumatoid arthritis, irritable bowel syndrome, systemic lupus erythematosus, multiple sclerosis, Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome and Sazary syndrome;
7. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
8. infectious diseases: virus diseases such as genital warts, common warts, plantar warts, hepatitis B, hepatitis C, herpes simplex virus, molluscum contagiosum, variola, human immunodeficiency virus (HIV), human papilloma virus (HPV), cytomegalovirus (CMV), varicella zoster virus (VZV), rhinovirus, adenovirus, coronavirus, influenza, para-influenza; bacterial diseases such as tuberculosis and mycobacterium avium, leprosy; other infectious diseases, such as fungal diseases, chlamydia, candida, aspergillus, cryptococcal meningitis, pneumocystis carnii, cryptosporidiosis, histoplasmosis, toxoplasmosis, trypanosome infection and leishmaniasis.

Thus, the present invention provides a compound of formula (I) or a pharmaceutically acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

In particular, the compounds of the invention (including pharmaceutically acceptable salts) may be used in the treatment of asthma {such as bronchial, allergic, intrinsic, extrinsic or dust asthma, particularly chronic or inveterate asthma (for example late asthma or airways hyper-responsiveness)}, chronic obstructive pulmonary disease (COPD) or allergic rhinitis.

The invention also provides a method of treating, or reducing the risk of, an obstructive airways disease or condition (e.g. asthma or COPD) which comprises administering to a patient in need thereof a therapeutically effective amount of a compound of formula (I) or a pharmaceutically acceptable salt thereof as hereinbefore defined.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). Alternatively, if the compound is administered orally, then the daily dosage of the compound of the invention may be in the range from 0.01 micrograms per kilogram body weight (μg/kg) to 100 milligrams per kilogram body weight (mg/kg).

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

Dry powder formulations and pressurized HFA aerosols of the compounds of the invention (that is, compounds of formula (I) and pharmaceutically acceptable salts thereof) may be administered by oral or nasal inhalation. For inhalation, the compound is desirably finely divided. The finely divided compound preferably has a mass median diameter of less than 10 micrometers (μm), and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention (that is, compounds of formula (I) and pharmaceutically is acceptable salts thereof) may also be administered in conjunction with other compounds used for the treatment of the above conditions.

The invention therefore further relates to combination therapies wherein a compound of the invention or a pharmaceutical composition or formulation comprising a compound of the invention is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with the following agents: non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention with a monoclonal antibody targeting B-Lymphocytes (such as CD20 (rituximab), MRA-aIL16R and T-Lymphocytes, CTLA4-Ig, HuMax I1-15).

The present invention still further relates to the combination of a compound of the invention with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention further relates to the combination of a compound of the invention with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY×1005.

The present invention further relates to the combination of a compound of the invention and a receptor antagonist for leukotrienes (LT) B4, LTC4, LTD4, and LTE4 selected from the group consisting of the phenothiazin-3-1 s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY×7195.

The present invention still further relates to the combination of a compound of the invention and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention and a proton pump inhibitor (such as omeprazole) or a gastroprotective histamine type 2 receptor antagonist.

The present invention further relates to the combination of a compound of the invention and an antagonist of the histamine type 4 receptor.

The present invention still further relates to the combination of a compound of the invention and an alpha-1/alpha-2 adrenoreceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention and an anticholinergic agents including muscarinic receptor (M1, M2, and M3) antagonist such as atropine, hyoscine, glycopyrrolate, ipratropium bromide, tiotropium bromide, oxitropium bromide, pirenzepine or telenzepine.

The present invention still further relates to the combination of a compound of the invention and a beta-adrenoreceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention and a chromone, such as sodium cromoglycate or nedocromil sodium.

The present invention further relates to the combination of a compound of the invention with an agent that modulates a nuclear hormone receptor such as PPARs.

The present invention still further relates to the combination of a compound of the invention together with an immunoglobulin (Ig) or Ig preparation or an antagonist or antibody modulating Ig function such as anti-IgE (for example omalizumab).

The present invention further relates to the combination of a compound of the invention and another systemic or topically-applied anti-inflammatory agent, such as thalidomide or a derivative thereof, a retinoid, dithranol or calcipotriol.

The present invention still further relates to the combination of a compound of the invention and combinations of aminosalicylates and sulfapyridine such as sulfasalazine, mesalazine, balsalazide, and olsalazine; and immunomodulatory agents such as the thiopurines.

The present invention further relates to the combination of a compound of the invention together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention and a cardiovascular agent such as a calcium channel blocker, a beta-adrenoreceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention and an agent for the treatment of acute or chronic pain, such as a centrally or peripherally-acting analgesic (for example an opioid or derivative thereof), carbamazepine, phenyloin, sodium valproate, amitryptiline or other anti-depressant agent-s, paracetamol, or a non-steroidal anti-inflammatory agent.

The present invention further relates to the combination of a compound of the invention together with a parenterally or topically-applied (including inhaled) local anaesthetic agent such as lignocaine or a derivative thereof.

A compound of the present invention can also be used in combination with an anti-osteoporosis agent including a hormonal agent such as raloxifene, or a biphosphonate such as alendronate.

The present invention still further relates to the combination of a compound of the invention together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sul rinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3 receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2X7; (xxvii) inhibitor of transcription factor activation such as NFkB, API, or STATS; or (xxviii) a glucocorticoid receptor agonist.

In a further aspect the present invention provides a (fixed dose) combination (for example for the treatment of COPD, asthma or allergic rhinitis) of a compound of formula (I) or a pharmaceutically acceptable salt thereof as hereinbefore defined, one or more agents independently selected from:
a selective β₂ adrenoreceptor agonist (such as metaproterenol, isoproterenol, isoprenaline, albuterol, salbutamol, formoterol, salmeterol, terbutaline, orciprenaline, bitolterol mesylate, pirbuterol or indacaterol);
a phosphodiesterase inhibitor (such as a PDE4 inhibitor);
a protease inhibitor (such as a neutrophil elastase or matrix metalloprotease MMP-12 inhibitor);
an anticholinergic agent;
a modulator of chemokine receptor function (such as a CCR1 receptor antagonist); and
an inhibitor of kinase function (such as the kinases p38 or IKK);
   and optionally one or more pharmaceutically acceptable excipients.

The invention also provides a pharmaceutical product comprising a preparation of a first active ingredient which is a compound of formula (I) or a pharmaceutically acceptable salt thereof as hereinbefore defined, and a preparation of a second active ingredient which is:
a selective β₂ adrenoreceptor agonist;
a phosphodiesterase inhibitor;
a protease inhibitor;
an anticholinergic agent;
a modulator of chemokine receptor function; or
an inhibitor of kinase function;
   wherein the preparations are for simultaneous, sequential or separate administration to a patient in need thereof.

In another aspect, the invention provides a kit comprising a preparation of a first active ingredient which is a compound of formula (I) or a pharmaceutically acceptable salt thereof as hereinbefore defined, and a preparation of a second active ingredient which is:
a selective β₂ adrenoreceptor agonist;
a phosphodiesterase inhibitor;
a protease inhibitor;
an anticholinergic agent;
a modulator of chemokine receptor function; or
an inhibitor of kinase function;
   and instructions for the simultaneous, sequential or separate administration of the preparations to a patient in need thereof.

A compound of the invention can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine is kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI-1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitrorcductasc enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The present invention will now be further explained by reference to the following illustrative examples in which the following abbreviations are used:
EtOAc ethyl acetate
HCl hydrochloric acid
H₂S hydrogen sulphide
CH₂Cl₂ dichloromethane (DCM)
DMF N,N-dimethylformamide
NaH sodium hydride
MgSO₄ magnesium sulfate
NaNO₂ sodium nitrite
K₂CO₃ potassium carbonate
SnCl₂ tin (II) chloride
NaOH sodium hydroxide
Na₂SO₄ sodium sulfate
NH₄Cl ammonium chloride
DIEA diisopropylethylamine
NMP N-methylpyrrolidone
DME dimethyl ether
DMSO dimethylsulfoxide
EtOH ethanol
THF tetrahydrofuran
TFA trifluoroacetic acid
HCl hydrochloric acid
DCM dichloromethane
NaHCO₃ sodium hydrogen carbonate
Et₃N triethylamine
MeOH methanol
MeCN/acetonitrile
CH₃CN
TBME tert-butyl methyl ether
EDTA ethylenediaminetetraacetic acid
cone. concentrated
rt room temperature
h hours
min minutes
M molar
MS mass spectrometry
APCI atmospheric chemical ionisation method
ESI electron spray ionisation method
NMR nuclear magnetic resonance
SCX solid phase extraction with a sulfonic acid sorbent
HPLC high performance liquid chromatography
LC-MS liquid chromatography with mass spectrometry detection

### General Methods

NMR spectra were recorded on a Varian Mercury-VX 300 MHz instrument or a Varian Inova 400 MHz instrument. The central peaks of chloroform-d (H 7.26 ppm), acetone-d₆ (H 2.05 ppm), acetonitrile-d₃ (δ_{H} 1.94 ppm) or DMSO-d₆ (H 2.50 ppm) were used as internal references.

The following method was used for LC/MS analysis:

Instrument Agilcnt 1100; Column Waters Symmetry 2.1×30 mm; Mass APCI; Flow rate 0.7 mL/min; Wavelength 254 nm; Solvent A: water+0.1% TFA; Solvent B: acetonitrile+0.1% TFA; Gradient 15-95%/B 2.7 min, 95% B 0.3 min.

Column chromatography was carried out using silica gel (0.040-0.063 mm, Merck).

For preparative HPLC either a Kromasil® KR-100-5-C18 column (250×20 mm, Akzo Nobel) and mixtures of acetonitrile/water (0.1% TFA) at a flow rate of 10 ml/min or a XTerra® Prep MS C₁₈ OBD™ Column, 5 μm, 19×50 mm (acetonitrile/water/0.1% NH₃) at a flow rate of 20 ml/min was used. UV=254 nm or 220 nm was used for detection.

Unless stated otherwise, starting materials were commercially available. All solvents and commercial reagents were of laboratory grade and were used as received.

### Intermediate 1

### (8S,9S,10R,11S,13S,14S,17R)-11,17-Dihydroxy-10,13-dimethyl-3-oxo-2,3,6,7,8,9,10,11,12,13,14,15,16,17-tetradecahydro-1H-cyclopenta[a]phenanthrene-17-carboxylic acid

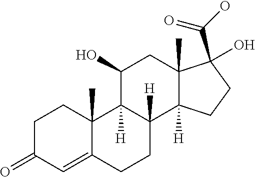

A solution of orthoperiodic acid (21.4 g, 94 mmol) in water (80 ml) was added to a solution of (8S,9S,10R,11S,13S,14S,17R)-11,17-dihydroxy-17-(2-hydroxyacetyl)-10,13-dimethyl-6,7,8,9,10,11,12,13,14,15,16,17-dodecahydro-1H-cyclopenta[a]phenanthren-3(2H)-one (17.0 g, 46.9 mmol) in THF (350 ml) and the reaction mixture was stirred at room temperature in an open flask for 2 h. The obtained mixture was poured onto ice and after the ice had molten, the mixture was extracted with ethyl acetate (3×150 ml). The combined organic fractions were concentrated in vacuo to yield white solid, which was dissolved in aq. NaOH (1 M, 150 ml). The aqueous solution was washed with ethyl acetate and acidified with conc. aqueous HCl. The obtained precipitate was collected by filtration and dried on the sinter in air overnight to give 15.51 g (95%) of the desired compound as an an off-white powder. APCI-MS m/z: 349 [MH⁺].

¹H NMR (400 MHz, DMSO-d₆) δ 12.20 (s, 1H), 5.55 (s, 1H), 4.74 (s, 1H), 4.24 (s, 2H), 2.43 (m, 3H), 2.18 (m, 2H), 2.09 (m, 1H), 2.00-1.44 (m, 6H), 1.37 (s, 3H), 1.31-1.15 (m, 1H), 0.98 (m, 1H), 0.89 (s, 3H), 0.83 (d, 1H).

### Intermediate 2

### (8S,9S,10R,11S,13S,14S,17R)-2-Formyl-11,17-dihydroxy-10,13-dimethyl-3-oxo-2,3,6,7,8,9,10,11,12,13,14,15,16,17-tetradecahydro-1H-cyclopenta[a]phenanthrene-17-carboxylic acid

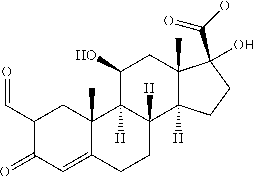

To a stirred suspension of sodium hydride (5.73 g, 143.5 mmol, 60% suspension in mineral oil) in THF (100 ml) under argon was added intermediate 1 (5.00 g, 14.35 mmol) in small portions at room temperature. After stirring for 5 minutes ethyl formate (58.4 ml, 717.5 mmol) was added and stirring was continued at room temperature overnight. Formic acid was added until no more gas evolution was observed, affording a thick suspension. 2M aq. NaOH solution (50 ml) was added and the obtained mixture was stirred for 10 min. The layers were separated, the aqueous layer was acidified with cone. aq. HCl and extracted with ethyl acetate (3×50 ml). The combined organic extracts were dried with sodium sulfate, filtered and the solvent was evaporated in vacuo to afford 5.65 g of the desired compound as a yellow foam which solidified. APCI-MS m/z: 377 [MH⁺].

¹H NMR (400 MHz, DMSO-d₆) δ 5.56 (s, 1H), 4.26 (m, 2H), 2.54-2.37 (m, 2H), 2.29-2.11 (m, 2H), 1.96-1.78 (m, 2H), 1.77-1.43 (m, 6H), 1.34-1.20 (m, 1H), 1.25 (s, 3H), 1.09-0.85 (m, 1H), 0.89 (s, 3H). APCI-MS m/z: 377 [MH⁺].

### Intermediate 3

### tert-Butyl 2-(3-(2-methoxyethylcarbamoyl)phenyl)hydrazinecarboxylate

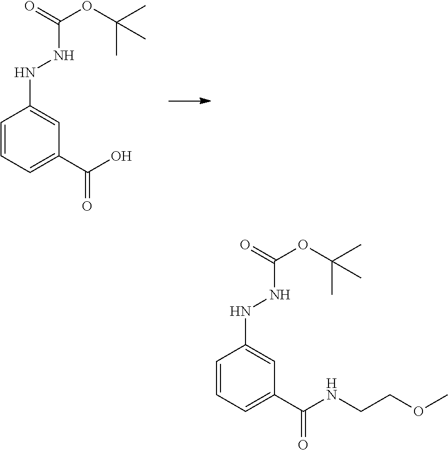 [SMILES: CC(C)(C)OC(=O)NNc1cccc(C(=O)O)c1 ; COCCNC(=O)c1cccc(NNC(=O)OC(C)(C)C)c1]

To a stirred solution of 3-(2-(tert-butoxycarbonyl)hydrazinyl)benzoic acid (505 mg, 2 mmol) in DMF (3 ml) was added di(1H-imidazol-1-yl)methanone (811 mg, 5 mmol) at room temperature. The mixture was stirred for 1 h and 2-Methoxyethanamine (451 mg, 6 mmol) was subsequently added. Stirring was continued overnight at the same temperature. The resulting mixture was poured into an aq. HCl solution (0.5 M, 25 ml) and extracted with EtOAc (2×25 ml). The combined organic extracts were washed with water and dried with Na₂SO₄. The drying agent was filtered off and the organic solution was concentrated in vacuo, resulting in a yellow oil which was purified by flash chromatography (silica gel, n-heptane/EtOAc, 50% to 90% gradient) to afford 446 mg (72%) of the target compound as a colourless oil which solidified slowly.

APCI-MS m/z: 310 [MH⁺].

¹H NMR (400 MHz, CDCl₃) δ 7.30-7.19 (3H, m), 6.95 (1H, ddd), 6.60 (1H, br.s), 6.53 (1H, br.s), 3.69 (3H, br.s), 3.63 (2H, m), 3.57 (2H, m), 3.39 3H, s), 1.47 (9H, s).

### Intermediate 4

### 3-Hydrazinyl-N-(2-methoxyethyl)benzamide

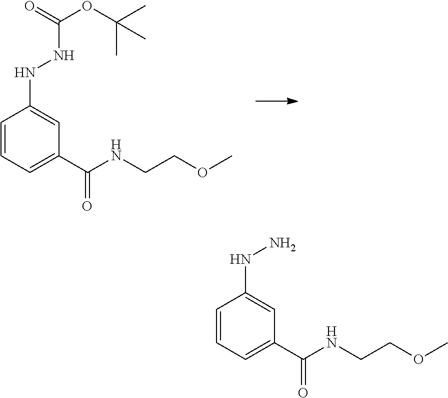 [SMILES: COCCNC(=O)c1cccc(NNC(=O)OC(C)(C)C)c1 ; COCCNC(=O)c1cccc(NN)c1]

To a stirred solution of intermediate 3 (440 mg, 1.42 mmol) in dichloromethane (10 ml) was added trifluoroacetic acid (2 ml). The mixture was stirred at room temperature for 1 h and the resulting mixture was extracted with water (25 ml). The aqueous extract was made alkaline by addition of aq. NaOH (40% wt.) and the product was extracted with EtOAc (3×15 ml). The combined organic extracts were dried with Na₂SO₄, the drying agent was filtered and the solvent was removed in vacuo to obtain 238 mg (80%) of a yellow oil which was used in the next step without any further purification.

APCI-MS m/z: 210 [MH⁺].

### Intermediate 5

### (1R,3aS,3bS,10aR,10bS,11S,12aS)-1,11-Dihydroxy-7-{3-[(2-methoxyethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazole-1-carboxylic acid

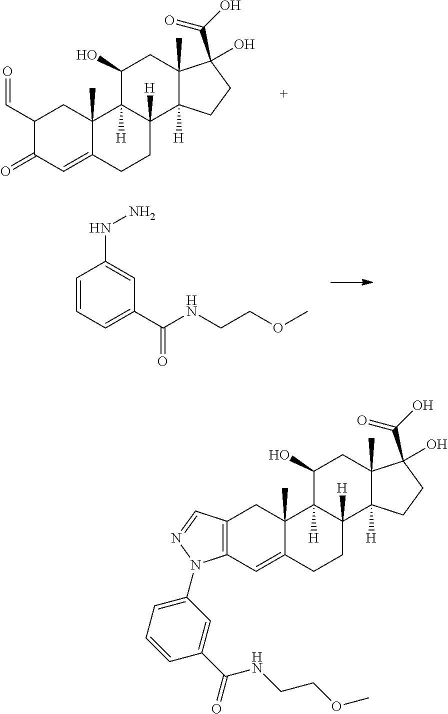

To a stirred solution of intermediate 2 (471 mg, 1.25 mmol) in acetic acid (10 ml) and water (2 ml) was added intermediate 4 (238 mg, 1.14 mmol) at room temperature. The mixture was stirred overnight and subsequently poured into water (100 ml). The resulting precipitate was collected by filtration and dried on the sinter in air to yield 455 mg (73%) of a yellow solid which was used as such without any further purification.

APCI-MS m/z: 550 [MH⁺].

### Intermediate 6

### (1R,3aS,3bS,10aR,10bS,11S,12aS)-7-{3-[(2-Methoxyethyl)carbamoyl]phenyl}-1,11-dihydroxy-10a,12a-dimethyl-1,2,3,3a,3b,4,5,7,10,10a,10b,11,12,12a-tetradecahydrocyclopenta[5,6]naphtho[1,2-f]indazole-1-carbothioic S-acid

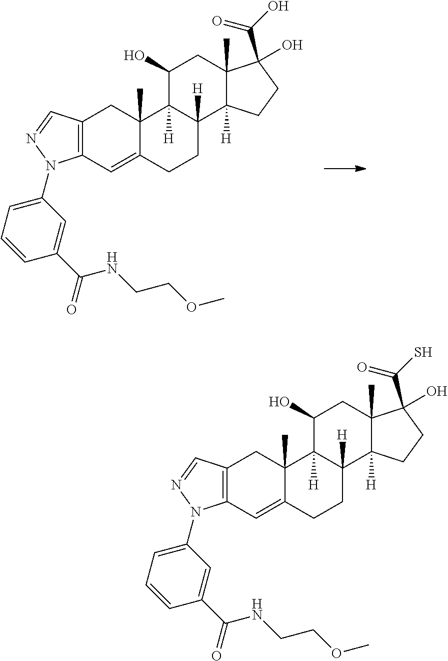

To a stirred solution of intermediate 5 (454 mg, 0.83 mmol) in DMF (5 ml) was added di(1H-imidazol-1-yl)methanone (335 mg, 2.07 mmol) at room temperature. The mixture was stirred for 3 h followed by bubbling through hydrogen sulfide gas through the stirred solution for 5 min. Stirring was continued for an additional 10 min. in a sealed flask and the mixture was subsequently poured into a mixture of ice (100 g) and aq. HCl (10 ml, 2 M). After the ice had melted, the resulting precipitate was collected by filtration and dried on a sinter in air to yield 431 mg (92%) the desired compound as a yellow solid.

APCI-MS m/z: 566 [MH⁺].

### Intermediate 7

### tert-Butyl 2-(3-(2-(methylthio)ethylcarbamoyl)phenyl)hydrazinecarboxylate

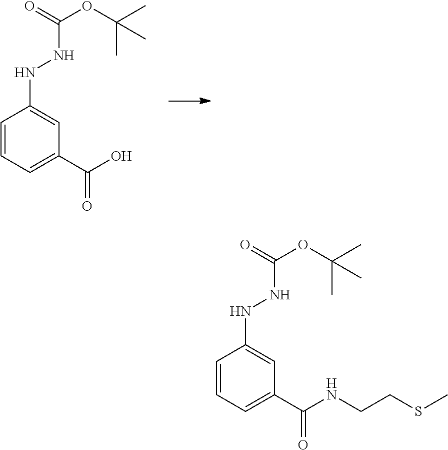 [SMILES: CC(C)(C)OC(=O)NNc1cccc(C(=O)O)c1 ; CSCCNC(=O)c1cccc(NNC(=O)OC(C)(C)C)c1]

The compound was prepared from 3-(2-(tert-butoxycarbonyl)hydrazinyl)benzoic acid according to the procedure described for intermediate 3.

APCI-MS m/z: 326 [MH⁺].

¹H NMR (400 MHz, CDCl₃) δ 7.30 (2H, m), 7.22 (1H, m), 6.97 (1H, dd), 6.60 (1H, br.s), 6.47 (1H, br.s), 3.66 (2H, q), 3.17 (1H, br.s), 2.76 (2H, t), 2.15 3H, s), 1.47 (9H, br.s).

### Intermediate 8

### 3-Hydrazinyl-N-(2-(methylthio)ethyl)benzamide

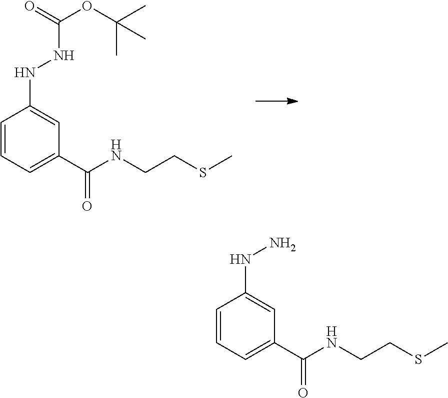 [SMILES: CSCCNC(=O)c1cccc(NNC(=O)OC(C)(C)C)c1 ; CSCCNC(=O)c1cccc(NN)c1]

The compound was prepared from intermediate 7 according to the procedure described for intermediate 4. APCI-MS m/z: 226 [MH⁺].

### Intermediate 9

### (1R,3aS,3bS,10aR,10bS,11S,12aS)-1,11-Dihydroxy-7-{3-[(2-(methylthio)ethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazole-1-carboxylic acid

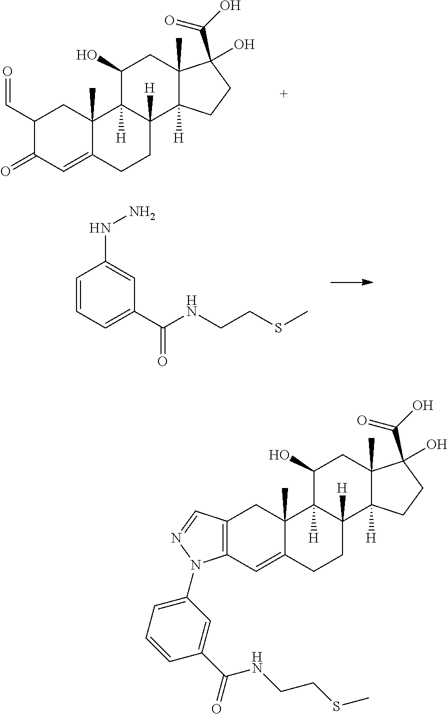

The compound was prepared from intermediate 2 and Intermediate 8 according to the procedure described for intermediate 5. APCI-MS m/z: 566 [MH⁺].

### Intermediate 10

### (1R,3aS,3bS,10aR,10bS,11S,12aS)-7-{3-[(2-(Methylthio)ethyl)carbamoyl]phenyl}-1,11-dihydroxy-10a,12a-dimethyl-1,2,3,3a,3b,4,5,7,10,10a,10b,11,12,12a-tetradecahydrocyclopenta[5,6]naphthol[1,2-f]indazole-1-carbothioic S-acid

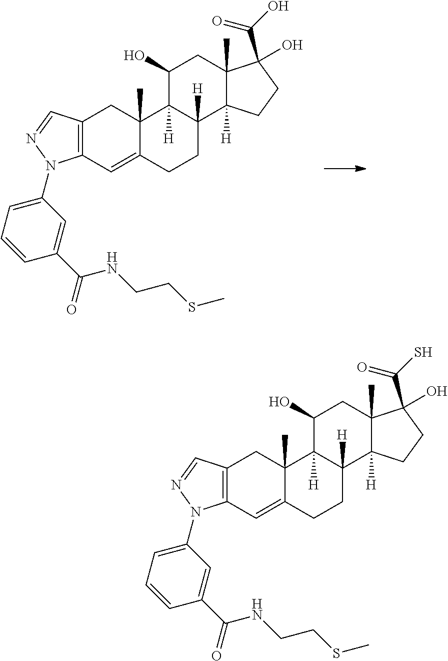

The compound was prepared from intermediate 9 according to the procedure described for intermediate 6. APCI-MS m/z: 582 [MH⁺].

### Intermediate 11

### (1R,3aS,3bS,10aR,10bS,11S,12aS)-7-(3-Carboxyphenyl)-1,11-dihydroxy-10a,12a-dimethyl-1,2,3,3a,3b,4,5,7,10,10a,10b,11,12,12a-tetradecahydrocyclopenta[5,6]naphthol[1,2-f]indazole-1-carboxylic acid

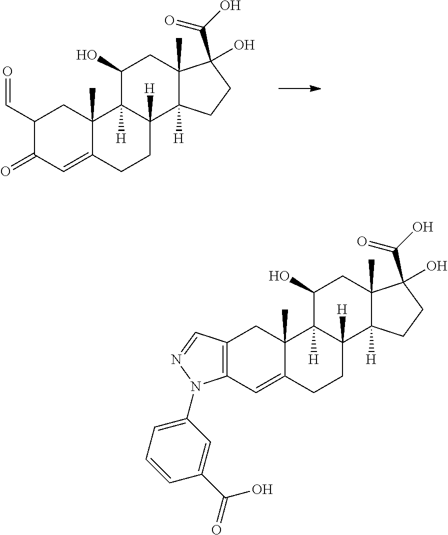

The compound was prepared from intermediate 2 and 3-hydrazinylbenzoic acid according to the procedure described for intermediate 5. APCI-MS m/z: 492 [MH⁺].

### Intermediate 12

### (1R,3aS,3bS,10aR,10bS,11S,12aS)-7-(3-Carboxyphenyl)-11-hydroxy-10a,12a-dimethyl-1-(propanoyloxy)-1,2,3,3a,3b,4,5,7,10,10a,10b,11,12,12a-tetradecahydrocyclopenta[5,6]naphtho[1,2-f]indazole-1-carboxylic acid

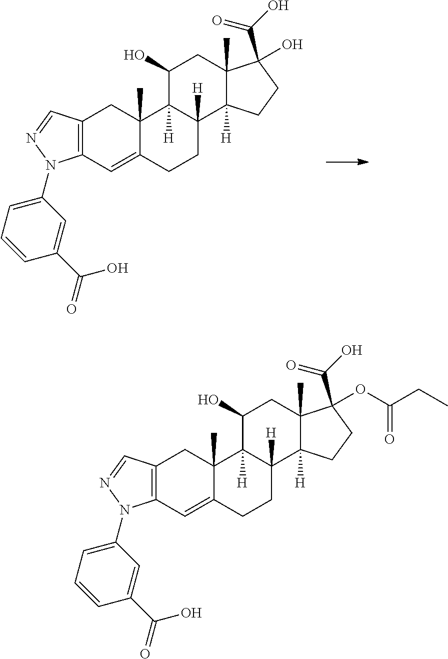

A stirred solution of intermediate 11 (919 mg, 1.87 mmol) and triethylamine (1.16 ml, 8.4 mmol) in dichloromethane (25 ml) was cooled to 0° C. under an argon atmosphere and a solution of propionyl chloride (691 mg, 7.46 mmol) in dichloromethane (5 ml) was added. The mixture was stirred for 1 h at the same temperature. N₁,N₁,N₂-Trimethylethane-1,2-diamine (0.95 ml, 7.46 mmol) was added and stirring was continued at room temperature for 30 min. The obtained mixture was diluted with dichloromethane (50 ml) and washed with aq. HCl (1M, 20 ml). An oily precipitate formed which was collected, dissolved in acetonitrile and dried with Na₂SO₄. Filtration of the drying agent followed by evaporation of the solvent in vacuo yielded 792 mg of the crude desired compound which was taken on as such without any further purification. APCI-MS m/z: 549 [MH⁺].

### Intermediate 13

### (1R,3aS,3bS,10aR,10bS,11S,12aS)-7-{3-[(2-Amino-2-oxoethyl)carbamoyl]phenyl}-11-hydroxy-10a,12a-dimethyl-1-(propanoyloxy)-1,2,3,3a,3b,4,5,7,10,10a,10b,11,12,12a-tetradecahydrocyclopenta[5,6]naphtho[1,2-f]indazole-1-carboxylic acid

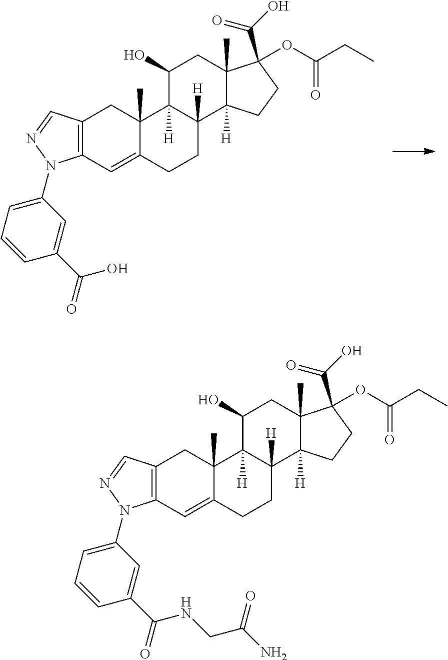

To a stirred solution of intermediate 12 (350 mg, 0.64 mmol) in DMF (10 ml) was added di(1H-imidazol-1-yl)methanone (259 mg, 1.59 mmol) and the mixture was stirred at room temperature for 5 h. 2-Aminoacetamide hydrochloride (212 mg, 1.91 mmol) was added, followed by triethylaminc (0.5 ml, mmol) and stirring was continued at the same temperature overnight. The obtained mixture was poured into aq. HCl solution (2 M, 100 ml) and extracted with EtOAc (2×50 ml). The combined organic extracts were washed with water (100 ml) and sat. aq. NaCl (50 ml) was added to the remaining aqueous layer, followed by extraction with EtOAc (50 ml). The combined organic extracts were dried over Na₂SO₄, filtered and the solvent was removed in vacuo. The resulting residue was dissolved in acetonitrile (2 ml)/water (0.5 ml), the resulting solution was purified by preparative HPLC and the product containing fractions were combined and freeze-dried to afford 102 mg (26%) of the desired ompound as yellowish solid. APCI-MS m/z: 605 [MH⁺].

### Intermediate 14

### (1R,3aS,3bS,10aR,10bS,11S,12aS)-7-{3-[(2-Amino-2-oxoethyl)carbamoyl]phenyl}-1-{[(dimethylcarbamoyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate

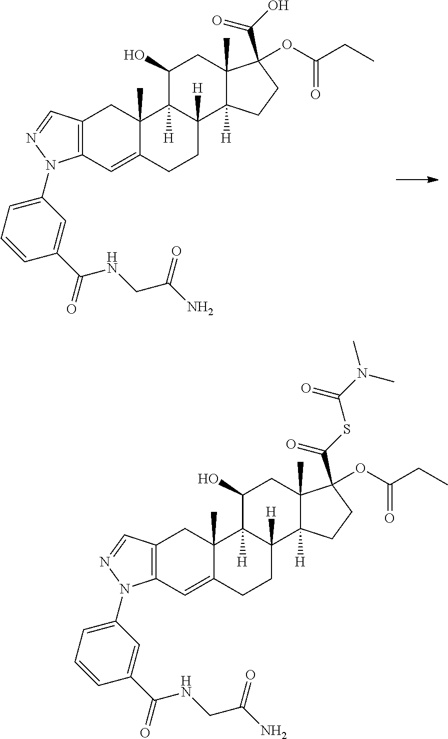

To a stirred solution of intermediate 13 (102 mg, 0.17 mmol) in acetone (5 ml) was added dimethylcarbamothioic chloride (62.5 mg, 0.51 mmol), followed by triethylamine (51.2 mg, 0.51 mmol), sodium iodide (5.06 mg, 0.03 mmol) and water (0.1 ml). Stirring was continued at room temperature overnight. N,N-Dimethylacetamide (1 ml) was added to the mixture followed by a second portion of dimethylcarbamothioic chloride (62.5 mg, 0.51 mmol) and stirring was continued for an additional 24 h. The obtained solution was poured into cold water (30 ml), acetone was removed in vacuo and the obtained precipitate was collected by filtration and dried on the sinter in air to afford 47 mg (40%) of the desired compound as a brownish solid. APCI-MS m/z: 692 [MH⁺].

### Intermediate 15

### (1R,3aS,3bS,10aR,10bS,11S,12aS)-7-{3-[(2-Amino-2-oxoethyl)carbamoyl]phenyl}-11-hydroxy-10a,12a-dimethyl-1-(propanoyloxy)-1,2,3,3a,3b,4,5,7,10,10a,10b,11,12,12a-tetradecahydrocyclopenta[5,6]naphtho[1,2-f]indazole-1-carbothioic S-acid

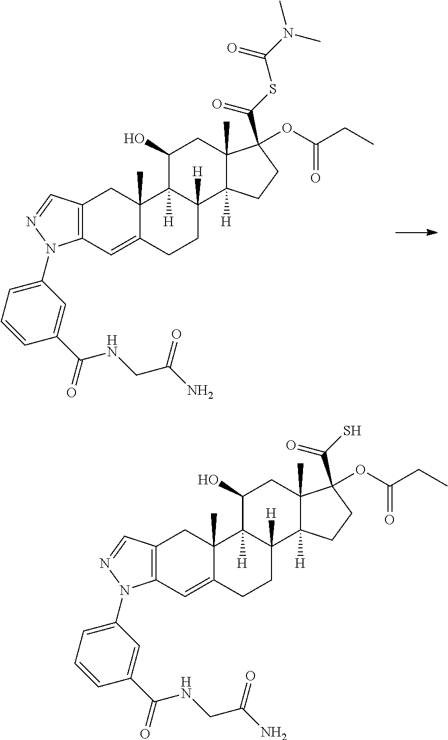

To a stirred suspension of intermediate 14 (47 mg, 70 μmol) in methanol (2 ml) was added potassium carbonate (18.8 mg, 140 μmol) and the resulting mixture was stirred at room temperature for 2 h. The obtained solution was poured into cold water (20 ml) and washed with toluene (20 ml). The aqueous layer was acidified with aq. HCl (2 M) and extracted with EtOAc (2×15 ml). The combined organic extracts were dried with Na₂SO₄, the drying agent was filtered and the solvent was removed in vacuo to afford 25 mg (59%) of the desired product as a brown oil. APCI-MS m/z: 621 [MH⁺].

### Intermediate 16

### (1R,3aS,3bS,10aR,10bS,11S,12aS)-7-{3-[(1,1-Dioxidotetrahydrothionhen-3-yl)carbamoyl]phenyl}-11-hydroxy-10a,12a-dimethyl-1-(propanoyloxy)-1,2,3,3a,3b,4,5,7,10,10a,10b,11,12,12a-tetradecahydrocyclopenta[5,6]naphtho[1,2-f]indazole-1-carboxylic acid

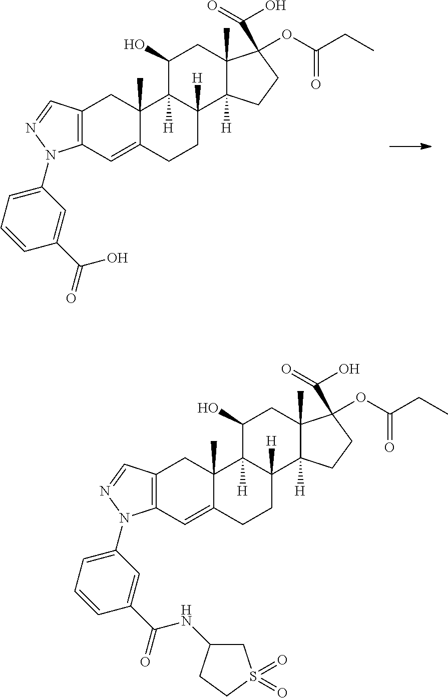

To a stirred solution of intermediate 12 (350 mg, 0.64 mmol) in DMF (5 ml) was added di(1H-imidazol-1-yl)methanone (259 mg, 1.59 mmol) and the resulting mixture was stirred at room temperature for 5 h. Tetrahydrophiophen-3-amine 1,1-dioxide (259 mg, 1.91 mmol) was added and stirring was continued at the same temperature overnight. The mixture was poured into aq. HCl (2 M, 100 ml), extracted with EtOAc (2×50 ml) and the combined organic extracts were washed with water (100 ml), dried with Na₂SO₄, filtered and the solvent was removed in vacuo. The crude material was dissolved in acetonitrile (2 ml)/water (0.5 ml), the resulting solution was purified by preparative HPLC and the product containing fractions were combined and freeze-dried to afford 110 mg (26%) of product as yellowish solid. APCI-MS m/z: 666 [MH⁺].

### Intermediate 17

### (1R,3aS,3bS,10aR,10bS,11S,12aS)-1-{[(Dimethylcarbamoyl)sulfanyl]carbonyl}-7-{3-[(1,1-dioxidotetrahydrothiophen-3-yl)carbamoyl]phenyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate

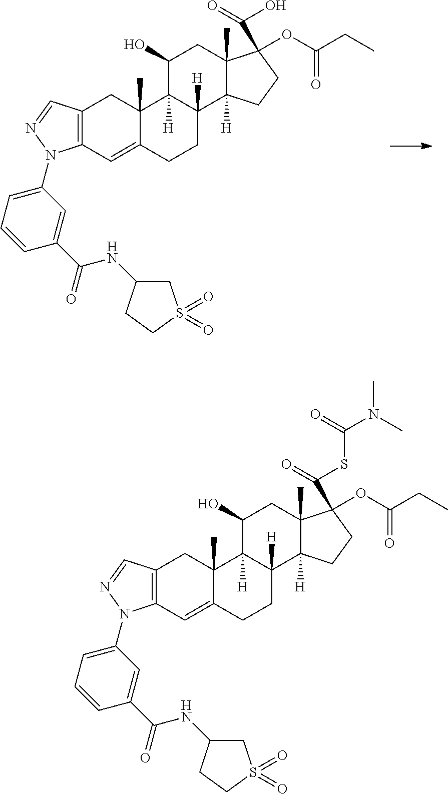

The compound was prepared from intermediate 6 according to the procedure described for intermediate 14. APCI-MS m/z: 753 [MH⁺].

### Intermediate 18

### (1R,3aS,3bS,10aR,10bS,11S,12aS)-7-{3-[(1,1-Dioxidotetrahydrothiophen-3-ylcarbamoyl]phenyl}-11-hydroxy-10a,12a-dimethyl-1-(propanoyloxy)-1,2,3,3a,3b,4,5,7,10,10a,10b,11,12,12a-tetradecahydrocyclopenta[5,6]naphtho[1,2-f]indazole-1-carbothioic S-acid

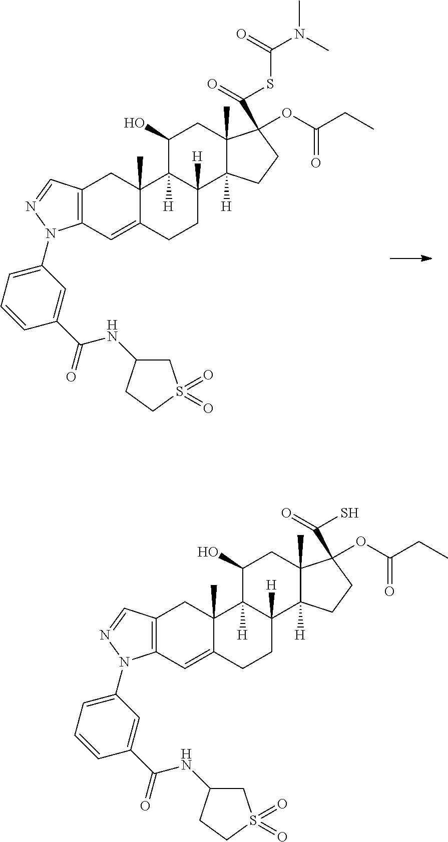

The compound was prepared from intermediate 7 according to the procedure described for intermediate 15. APCI-MS m/z: 682 [MH⁺].

### Intermediate 19

### tert-Butyl 2-(3-{[(2R)-2-carbamoylpyrrolidin-1-yl]carbonyl}phenyl)-hydrazinecarboxylate

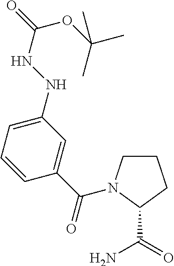 [SMILES: CC(C)(C)OC(=O)NNc1cccc(C(=O)N2CCC[C@@H]2C(N)=O)c1]

3-(2-Tert-butoxycarbonyl)hydrazinyl)benzoic acid (1.96 g, 7.77 mmol) was dissolved in NMP (10 ml) and 2-(3H-[1,2,3]triazolo[4,5-b]pyridin-3-yl)-1,1,3,3-tetramethylisouronium hexafluorophosphate(V) (5.91 g, 15.55 mmol), N-ethyl-N-isopropylpropan-2-amine (4.1 ml, 23.32 mmol) and (R)-pyrrolidine-2-carboxamide (0.89 g, 7.77 mmol) were added at room temperature. Stirring was continued overnight, the mixture was poured into aqueous HCl (˜0.5 M) and the product was extracted with EtOAc (3 times 100 ml). The combined organic extracts were washed with aqueous NaHCO₃, brine and dried with Na₂SO₄. The crude product (1.3 g) was obtained after filtration and evaporation of the solvent in vacuo and was used in the next step without any further purification. APCI-MS m/z: 293 [MH⁺].

### Intermediate 20

### 1-[(3-Hydrazinophenyl)carbonyl]-D-prolinamide trifluoracetic acid

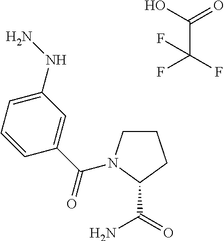 [SMILES: NNc1cccc(C(=O)N2CCC[C@@H]2C(N)=O)c1 ; O=C(O)C(F)(F)F]

Intermediate 19 (1.3 g, 3.73 mmol) was dissolved in CH₂Cl₂ (30 ml) and TFA (8 ml) and stirring was continued for 40 min. at room temperature. The solvent was removed in vacuo. CH₂Cl₂ and toluene were added and concentration of the solution in vacuo yielded the crude product as a yellow oil which was used in the next step without any further purification. APCI-MS m/z: 249 [MH⁺].

### Intermediate 21

### (1R,3aS,3bS,10aR,10bS,11S,12aS)-7-(3-{[(2R)-2-Carbamoylpyrrolidin-1-yl]carbonyl}phenyl)-1,11-dihydroxy-10a,12a-dimethyl-1,2,3,3a,3b,4,5,7,10,10a,10b,11,12,12a-tetradecahydrocyclopenta[5,6]naphtho[1,2-f]indazole-1-carboxylic acid

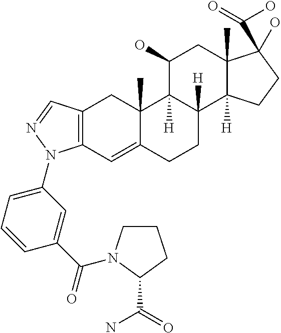

To a stirred ice-cold solution of intermediate 20 (1.4 g, 3.73 mmol) in acetic acid (15 ml) and water (3 ml) was added intermediate 2 (0.93 g, 3.73 mmol) and potassium acetate (0.73 g, 7.46 mmol). The mixture was warmed up to room temperature and stirring was continued for 2 hours. The mixture was poured into water (200 ml) and the resulting precipitate was filtered off, washed with water and dried on the sinter in air to yield 1.04 g of the desired compound as a solid which was used in the next step without any further purification. APCI-MS m/z: 589 [MH⁺].

### Intermediate 22

### (1R,3aS,3bS,10aR,10bS,11S,12aS)-7-(3-{[(2R)-2-Carbamoylpyrrolidin-1-yl]carbonyl}phenyl)-1,11-dihydroxy-10a,12a-dimethyl-1,2,3,3a,3b,4,5,7,10,10a,10b,11,12,12a-tetradecahydrocyclopenta[5,6]naphtho[1,2-f]indazole-1-carbothioic S-acid

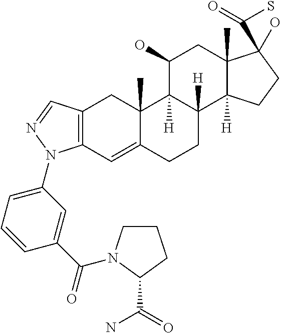

Intermediate 21 (1.04 g, 1.77 mmol) was dissolved in DMF (10 ml) and di(1H-imidazol-1-yl)methanone (0.58 g, 3.55 mmol) was added at room temperature. The mixture was stirred overnight. H₂S was bubbled through for a few minutes and the mixture was stirred for an additional 15 min. The mixture was poured into 1 M HCl and a yellow precipitate was formed which was filtered off, washed with water and dried on the sinter in air to yield 0.84 g of the desired compound as a solid which was taken on as such in the next step without any further purification. APCI-MS m/z: 605 [M⁺].

### Intermediate 23

### (8S,9R,10S,11S,13S,14S,17R)-9-Fluoro-11,17-dihydroxy-10,13-dimethyl-3-oxo-2,3,6,7,8,9,10,11,12,13,14,15,16,17-tetradecahydro-1H-cyclopenta[a]phenanthrene-17-carboxylic acid

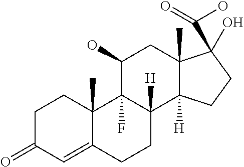

In a 1000 mL round-bottomed flask was suspended 2-((8S,9R,10S,11S,13S,14S,17R)-9-fluoro-11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxoethyl acetate (Fludrocortisone-21-acetate, 22.8 g, 53.97 mmol) in MeOH (200 mL) and the suspension was degassed with argon. 2M sodium hydroxide (40.5 mL, 80.95 mmol) was added to the solution and the mixture was stirred for 10 minutes. To the solution was added 4M HCl (20 ml, 80 mmol) and MeOH was removed in vacuo. The obtained residue was dissolved in THF (200 ml), a solution of orthoperiodic acid (15.99 g, 70.16 mmol) in water (40 ml) was added at room temperature and the obtained mixture was stirred for 1 hour. 100 ml of water was added and the organic solvent was removed in vacuo. An additional 100 ml of water was added to the aqueous residue and the obtained solid was collected by filtration, was washed with water (2×200 ml) and was air dried on the sinter, followed by drying in vacuo to yield 20 g of the desired compound as an off-white solid. APCI-MS m/z: 367 [MH⁺].

### Intermediate 24

### (8S,9R,10S,11S,13S,14S,17R,Z)-9-Fluoro-11,17-dihydroxy-2-(hydroxymethylene)-10,13-dimethyl-3-oxo-2,3,6,7,8,9,10,11,12,13,14,15,16,17-tetradecahydro-1H-cyclopenta[a]phenanthrene-17-carboxylic acid

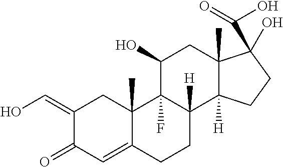

To a stirred suspension of sodium hydride (6.55 g, 272.91 mmol) (10.9 g, 60% suspension in mineral oil) in THF (130 mL) was added intermediate 23 (10 g, 27.29 mmol) in 2-3 portions followed by ethyl formate (111 mL, 1364.54 mmol). The mixture was stirred at room temperature for approximately 2 hours in an argon atmosphere. The reaction was quenched by careful addition of 2M NaOH (50 ml) and the phases were separated. The organic phase was extracted with an additional 2×20 ml of 2M NaOH. The combined aqueous solutions were diluted with water (15 ml), washed with Et₂O (40 ml) and acidified by addition of 4M HCl. The product was extracted with EtOAc (3×100 ml) and the combined organic phases were washed with brine (30 ml), dried over Na₂SO₄, filtered and evaporated in vacuo to give 8.6 g of the desired product as an orange semi-solid which was used directly in the next step without any further purification. APCI-MS m/z: 395 [MH⁺].

### Intermediate 25

### (1R,3aS,3bS,10aS,10bR,11S,12aS)-7-(3-{[(2R)-2-Carbamoylpyrrolidin-1-yl]carbonyl}phenyl)-10b-fluoro-1,11-dihydroxy-10a,12a-dimethyl-1,2,3,3a,3b,4,5,7,10,10a,10b,11,12,12a-tetradecahydrocyclopenta[5,6]naphtho[1,2-f]indazole-1-carboxylic acid

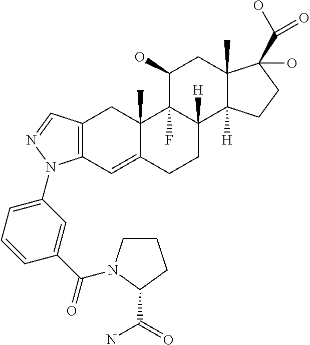

The compound was prepared according to the procedure for intermediate 21, starting from intermediate 24 and intermediate 20. APCI-MS m/z: 607 [MITI.

### Intermediate 26

### 1R,3aS,3bS,10aS,10bR,11S,12aS)-7-(3-{1(2R)-2-Carbamoylpyrrolidin-1-yl]carbonyl}phenyl)-10b-fluoro-1,11-dihydroxy-10a,12a-dimethyl-1,2,3,3a,3b,4,5,7,10,10a,10b,11,12,12a-tetradecahydrocyclopenta[5,6]naphtho[1,2-f]indazole-1-carbothioic S-acid

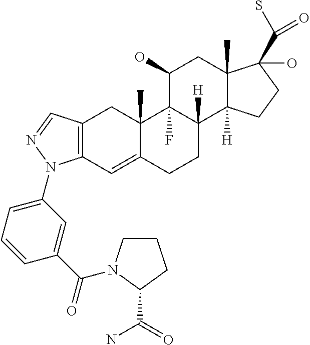

The compound was prepared from intermediate 25 according to the same procedure as for intermediate 22. APCI-MS m/z: 623 [MH⁺].

### EXAMPLE 1

### (1R,3aS,3bS,10aRJObS,11S,12aS)-1-{[(Cyanomethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazol-1-ylpropanoate

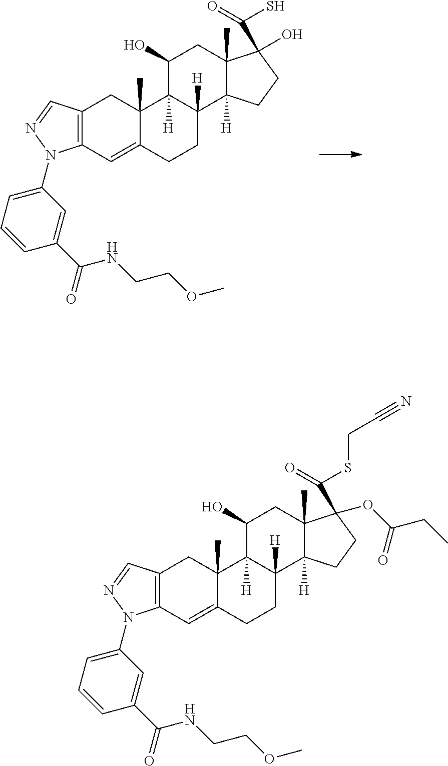

A stirred solution of intermediate 6 (86 mg, 0.15 mmol) and triethylamine (84 μl, 0.61 mmol) in DCM (10 ml) was cooled to 0° C. in an argon atmosphere and a solution of propionyl chloride (42 mg, 0.46 mmol) in DCM (2 ml) was added. The mixture was stirred for 1 h at the same temperature, followed by addition of N₁,N₁,N₂-trimethylethane-1,2-diamine (58 μl, 0.46 mmol). Stirring was continued at the same temperature for 30 min. and 2-bromoacetonitrile (73 mg, 0.61 mmol) was subsequently added. The cooling bath was removed and stirring was continued for 1 h whilst warming up to room temperature. The reaction mixture was diluted with DCM (10 ml), washed with aq. HCl (1M, 10 ml) and water (10 ml) and the organic layer was dried with Na₂SO₄. After filtration and evaporation of the solvent in vacuo the crude product dissolved in acetonitrile (2 ml)/water (0.5 ml), the resulting solution was purified by preparative HPLC and the product containing fractions were combined and freeze-dried to afford 12 mg (12%) of the desired product as a yellowish solid.

APCI-MS m/z: 661 [MH⁺].

¹H NMR (400 MHz, CDCl₃) δ 8.05 (1H, s), 7.83 (1H, d), 7.58 (3H, m), 6.98 (1H, br.s), 6.17 (1H, s), 4.57 (1H, m), 3.79 (1H, d), 3.68 (2H, q), 3.58 (3H, m), 3.41 (3H, s), 3.06 (1H, d), 2.98 (1H, dd), 2.75 (1H, d), 2.54 (1H, m), 2.38 (5H, m), 2.21-1.39 (17H, m), 1.35 (3H, s), 1.28 (1H, d), 1.15 (4H, m), 1.01 (3H, s), 0.99 (1H, m).

### EXAMPLE 2

### (1R,3aS,3bS,10aR,10bS,11S,12aS)-1-{[(Fluoromethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazol-1-yl propanoate

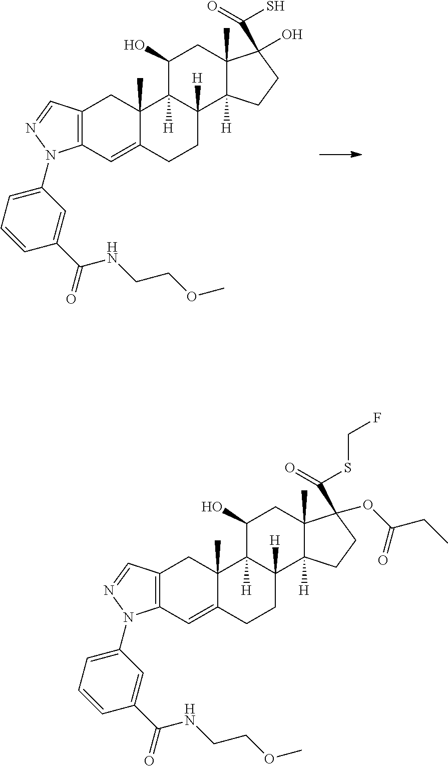

The compound was prepared from intermediate 6 and bromofluoromethane according to the procedure described in Example 1.

APCI-MS m/z: 654 [MH⁺].

¹H NMR (400 MHz, CDCl₃) δ 0.03 (1H, s), 7.82 (1H, d), 7.64-7.51 (3H, m), 6.87 (1H, br.s), 6.16 (1H, s), 5.96 (1H, dd), 5.70 (1H, dd), 4.54 (1H, m), 3.68 (2H, q), 3.59 (2H, t), 3.40 (3H, s), 3.05 (1H, d), 3.00 (1H, m), 2.53 (1H, d), 2.47-1.40 (17H, m), 1.34 (3H, s), 1.28 (1H, m), 1.22-1.06 (4H, m), 1.00 (3H, s), 0.98 (1H, m).

### EXAMPLE 3

### (1R,3aS,3bS,10aR,10bS,11S,12aS)-1-{[(Fluoromethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazol-1-yl methoxyacetate

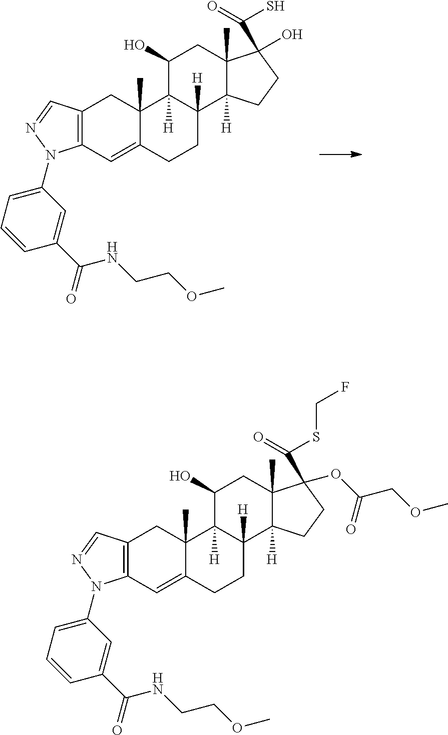

The compound was prepared from intermediate 6, 2-methoxyacetyl chloride and bromofluoromethane according to the procedure described in Example 1.

APC₁-MS m/z: 670 [MH⁺].

¹H NMR (400 MHz, CDCl₃) δ 8.21 (1H, br.s), 7.92 (2H, br.s), 7.70-7.50 (3H, m), 7.37 (1H, br.s), 6.17 (1H, s), 5.96 (1H, dd), 5.72 (1H, dd), 4.55 (1H, m), 4.10 (1H, m), 4.07 (2H, s), 3.68 (2H, t), 3.65-3.56 (2H, m), 3.47 (1H, m), 3.41 (3H, s), 3.40 (3H, m), 3.10 (1H, d), 3.04 (1H, m), 2.76 (1H, d), 2.58 (2H, m), 2.39 (1H, d), 2.16-1.97 (6H, m), 1.83 (2H, m), 1.70-1.41 (3H, m), 1.36 (3H, s), 1.29 (1H, m), 1.16 (1H, m), 1.02 (3H, s), 0.99 (1H, m).

### EXAMPLE 4

### (1R,3aS,3bS,10aR,10bS,11S,12aS)-1-{[(Cyanomethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazol-1-yl cyclopropanecarboxylate

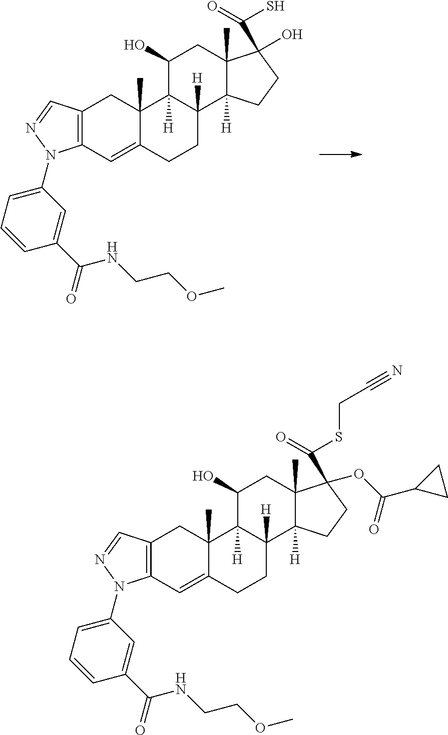

The compound was prepared from intermediate 6 and cyclopropanecarbonyl chloride and 2-bromoacetonitrile according to the procedure described in Example 1.

APCI-MS m/z: 673 [MH⁺].

¹H NMR (400 MHz, CDCl₃) δ 8.13 (1H, s), 7.87 (1H, d), 7.64-7.51 (3H, m), 7.14 (1H, br.s), 6.17 (1H, s), 4.58 (1H, m), 3.78 (1H, d), 3.68 (2H, q), 3.61 (2H, t), 3.56 (1H, d), 3.41 (3H, s), 3.09 (1H, d), 2.96 (1H, dd), 2.76 (1H, d), 2.57 (1H, m), 2.37 (1H, m), 2.26-1.80 (10H, m), 1.67 (2H, m), 1.47 (2H, m), 1.36 (3H, s), 1.28 (1H, m), 1.20-0.90 (7H, m).

### EXAMPLE 5

### (1R,3aS,3bS,10aR,10bS,11S,12aS)-1-{[(Fluoromethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazol-1-yl cyclopropanecarboxylate

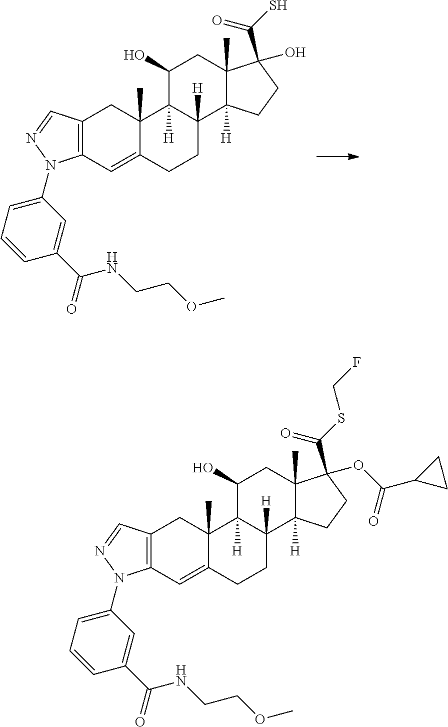

The compound was prepared from intermediate 6, cyclopropanecarbonyl chloride and bromofluoromethane according to the procedure described in Example 1.

APCI-MS m/z: 667 [MH⁺].

¹H NMR (400 MHz, CDCl₃) δ ¹H NMR (400 MHz, CDCl₃) δ 8.03 (1H, s), 7.82 (1H, d), 7.64-7.52 (3H, m), 6.86 (1H, br.s), 6.17 (1H, s), 5.96 (1H, dd), 5.71 (1H, dd), 4.57 (1H, m), 3.68 (2H, q), 3.59 (2H, t), 3.40 (3H, s), 3.05 (1H, d), 2.98 (1H, m), 2.75 (1H, d), 2.53 (1H, m), 2.34 (1H, d), 2.19-1.64 (10H, m), 1.46 (2H, m), 1.34 (3H, s), 1.29 (1H, dd), 1.21-0.88 (7H, m).

### EXAMPLE 6

### (1R,3aS,3bS,10aR,10bS,11S,12aS)-1-{[(Cyanomethyl)sulfanyl]carbonyl}-11-hydroxy-10a,12a-dimethyl-7-(3-{[2-(methylsulfanyl)ethyl]carbamoyl}phenyl)-1,2,3,3a,3b,4,5,7,10,10a,10b,11,12,12a-tetradecahydrocyclopenta[5,6]naphtho[1,2-f]indazol-1-yl propanoate

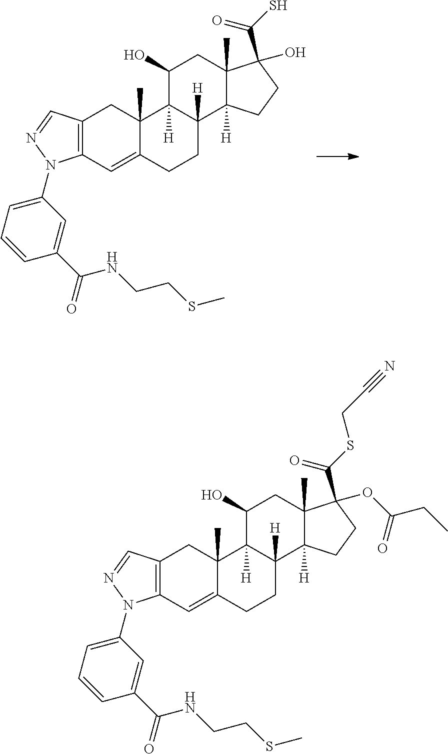

The compound was prepared from intermediate 10, propionyl chloride and bromofluoromethane according to the procedure described in Example 1.

APCI-MS m/z: 678 [MH⁺].

¹H NMR (400 MHz, CDCl₃) δ 7.93 (1H, s), 7.76 (1H, d), 7.66 (1H, m), 7.55 (1H, t), 7.47 (1H, s), 6.66 (1H, t), 6.16 (1H, s), 4.57 (1H, m), 3.78 (1H, d), 3.69 (2H, q), 3.57 (1H, d), 3.02 (1H, d), 2.97 (1H, m), 2.78 (2H, t), 2.73 (1H, d), 2.52 (1H, m), 2.39 (2H, q), 2.32 (1H, m), 2.16 (3H, s), 2.14-1.93 (6H, m), 1.82 (1H, m), 1.64 (2H, m), 1.47 (2H, m), 1.33 (3H, s), 1.27 (1H, dd), 1.16-1.04 (95H, m), 1.01 (3H, s).

### EXAMPLE 7

### (1R,3aS,3bS,10aR,10bS,11S,12aS)-1-{[(Fluoromethyl)sulfanyl]carbonyl}-11-hydroxy-(10a,12a-dimethyl-7-(3-{[2-(methylsulfanyl)ethyl]carbamoyl}phenyl)-1,2,3,3a,3b,4,5,7,10,10a,10b,11,12,12a-tetradecahydrocyclopenta[5,6]naphtho[1,2-f]indazol-1-yl propanoate

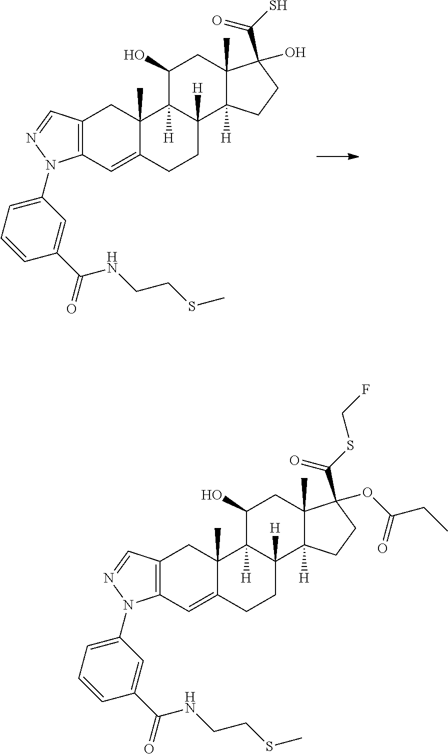

The compound was prepared from intermediate 10, propionyl chloride and bromofluoromethane according to the procedure described in Example 1.

APCI-MS m/z: 670 [MH⁺].

¹H NMR (400 MHz, CDCl₃) δ 7.93 (1H, s), 7.77 (1H, d), 7.66 (1H, d), 7.55 (1H, t), 7.47 (1H, s), 6.67 (1H, m), 6.16 (1H, s), 5.96 (1H, dd), 5.71 (1H, dd), 4.57 (1H, d), 3.70 (2H, q), 3.00 (2H, m), 2.78 (2H, t), 2.74 (1H, d), 2.51 (1H, m), 2.39 (1H, qd), 2.31 (1H, m), 2.16 (3H, s), 2.13-1.92 (5H, m), 1.82 (1H, m), 1.66 (1H, m), 1.46 (2H, m), 1.33 (3H, s), 1.28 (1H, dd), 1.17 (6H, m), 1.00 (3H, s).

### EXAMPLE 8

### (1R,3aS,3bS,10aR,10bS,11S,12aS)-1-{[(Fluoromethyl)sulfanyl]carbonyl}-11-hydroxy-10a,12a-dimethyl-7-(3-{[2-(methylsulfanyl)ethyl]carbamoyl}phenyl)-1,2,3,3a,3b,4,5,7,10,10a,10b,11,12,12a-tetradecahydrocyclopenta[5,6]naphtho[1,2-f]indazol-1-yl methoxyacetate

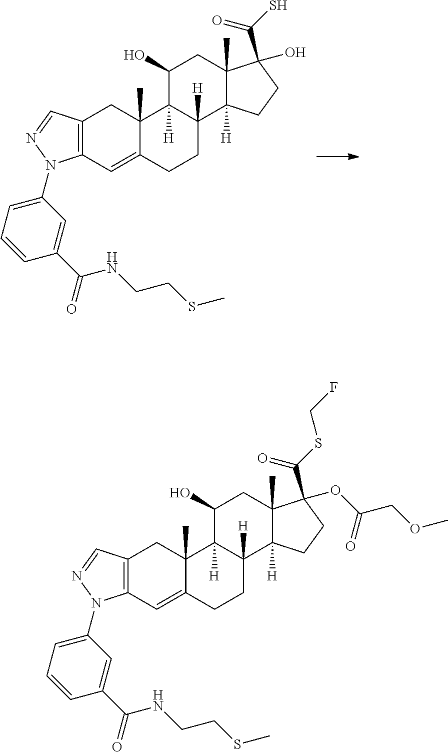

The compound was prepared from intermediate 10, 2-methoxyacetyl chloride and bromofluoromethane according to the procedure described in Example 1.

APCI-MS m/z: 686 [MH⁺].

¹H NMR (400 MHz, CDCl₃) δ 7.93 (1H, s), 7.77 (1H, d), 7.65 (1H, d), 7.57 (1H, t), 7.47 (1H, s), 6.65 (1H, m), 6.17 (1H, s), 5.94 (1H, dd), 5.74 (1H, dd), 4.57 (1H, m), 4.08 (2H, s), 3.70 (2H, q), 3.47 (3H, s), 3.03 (2H, m), 2.78 (2H, t), 2.73 (1H, d), 2.51 (1H, m), 2.32 (1H, m), 2.16 (3H, s), 2.15-1.93 (6H, m), 1.83 (1H, m), 1.65 (1H, m), 1.47 (2H, m), 1.33 (3H, s), 1.26 (1H, d), 1.18-1.04 (73H, m), 1.01 (3H, s).

### EXAMPLE 9

### (1R,3aS,3bS,10aR,10bS,11S,12aS)-1-{[(Cyanomethyl)sulfanyl]carbonyl}-11-hydroxy-10a,12a-dimethyl-7-(3-{[2-(methylsulfanyl)ethyl]carbamoyl}phenyl)-1,2,3,3a3b,4,5,7,10,10a,10b,11,12,12a-tetradecahydrocyclopenta[5,6]naphtho[1,2-f]indazol-1-yl cyclopropanecarboxylate

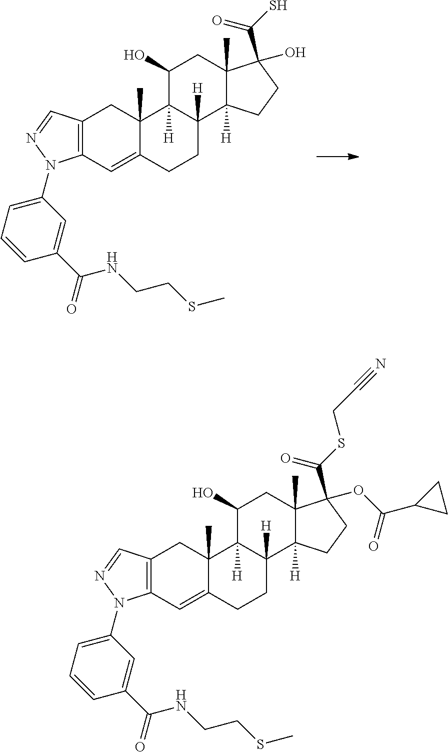

The compound was prepared from intermediate 10 and cyclopropanecarbonyl chloride and bromofluoromethane according to the procedure described in Example 1.

APCI-MS m/z: 690 [MH⁺].

¹H NMR (400 MHz, CDCl₃) δ 7.93 (1H, s), 7.77 (1H, d), 7.66 (1H, d), 7.55 (1H, t), 7.47 (1H, s), 6.68 (1H, t), 6.17 (1H, s), 4.58 (1H, d), 3.77 (1H, d), 3.69 (2H, q), 3.57 (1H, d), 3.03 (1H, d), 2.95 (1H, m), 2.78 (2H, t), 2.74 (1H, d), 2.52 (1H, m), 2.31 (1H, m), 2.16 (3H, s), 2.15-1.92 (5H, m), 1.84 (1H, m), 1.72-1.63 (2H, m), 1.47 (1H, m), 1.33 (3H, s), 1.31-0.91 (11H, m).

### EXAMPLE 10

### (1R,3aS,3 bS,10aR,10bS,11S,12aS)-1-{[(Fluoromethyl)sulfanyl]carbonyl}-11-hydroxy-10a,12a-dimethyl-7-(3-{[2-(methylsulfanyl)ethyl]carbamoyl}phenyl)-1,2,3,3a,3b,4,5,7,10,10a,10b,11,12,12a-tetradecahydrocyclopenta[5,6]naphtho[1,2-f]indazol-1-yl cyclopropanecarboxylate

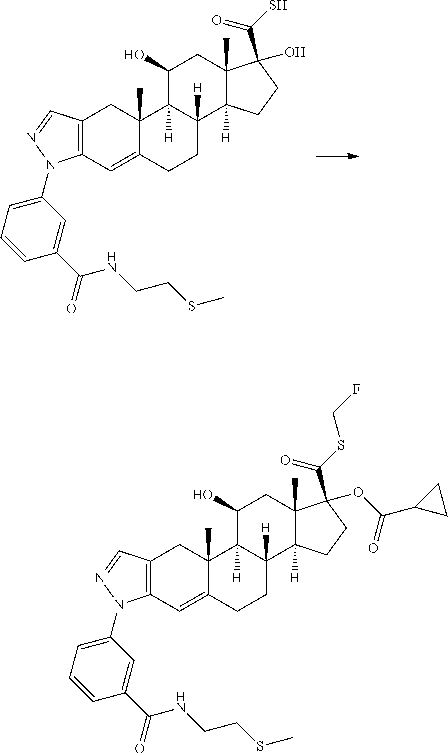

The compound was prepared from intermediate 10, cyclopropanecarbonyl chloride and bromofluoromethane according to the procedure described in Example 1.

APCI-MS m/z: 683 [MH⁺].

¹H NMR (400 MHz, CDCl₃) δ 7.93 (1H, s), 7.77 (1H, d), 7.66 (1H, d), 7.55 (1H, t), 7.47 (1H, s), 6.67 (1H, t), 6.17 (1H, s), 5.96 (1H, dd), 5.71 (1H, dd), 4.57 (1H, s), 3.69 (2H, q), 3.03 (1H, d), 2.98 (1H, m), 2.78 (2H, t), 2.73 (1H, d), 2.51 (1H, m), 2.32 (1H, m), 2.16 (3H, s), 2.14 (1H, m), 2.09-1.90 (4H, m), 1.83 (1H, m), 1.68 (2H, m), 1.46 (1H, m), 1.33 (43H, s), 1.28 (1H, dd), 1.20-0.87 (11H, m).

### EXAMPLE 11

### (1R,3aS,3bS,10aR,10bS,11S,12aS)-7-{3-[(2-Amino-2-oxoethyl)carbamoyl]phenyl}-1-{[(cyanomethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2f]indazol-1-yl propanoate

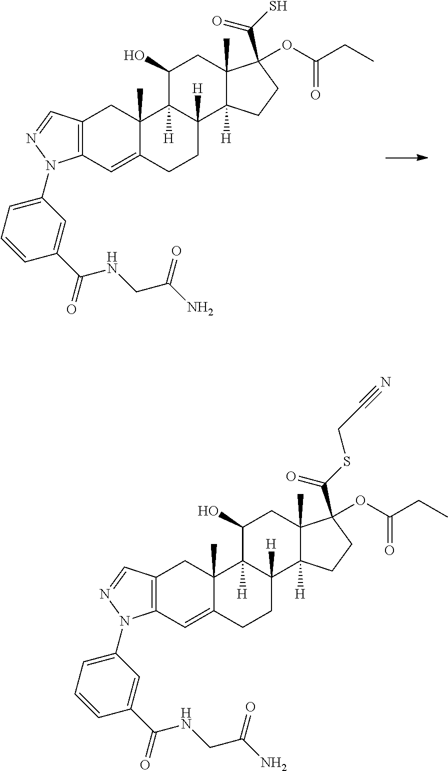

To a stirred solution of intermediate 15 (25 mg, 40 μmol) in dichloromethane (2 ml) and triethylamine (200 μl) was added 2-bromoacetonitrile (14.5 mg, 120 μmol) at room temperature and stirring was continued overnight. The mixture was concentrated in vacuo, the residue was dissolved in acetonitrile (2 ml)/water (0.5 ml) and the product was purified by preparative HPLC. The product containing fractions were combined and freeze-dried to give 2 mg (8%) of the desired product as a white solid.

APCI-MS m/z: 660 [MH⁺].

¹H NMR (400 MHz, CDCl₃) δ 8.00 (1H, s), 7.80 (1H, d), 7.66 (1H, d), 7.55 (1H, t), 7.46 (1H, s), 7.23 (4H, t), 6.14 (2H, s), 5.65 (1H, br.s), 4.57 (1H, d), 4.17 (2H, d), 3.79 (1H, d), 3.57 (1H, d), 3.01 (1H, d), 2.97 (1H, m), 2.72 (1H, d), 2.51 (1H, m), 2.39 (2H, q), 2.31 (1H, m), 2.11 (1H, dd), 2.07-1.92 (3H, m), 1.83 (1H, m), 1.64 (3H, m), 1.47 (1H, m), 1.32 (43H, s), 1.26 (1H, m), 1.16 (3H, t), 1.10 (1H, m), 1.01 (3H, s).

### EXAMPLE 12

### (1R,3aS,3bS,10aR,10bS,11S,12aS)-1-{[(Cyanomethyl)sulfanyl]carbonyl}-7-{3-[(1,1-dioxidotetrahydrothiophen-3-yl)carbamoyl]phenyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate

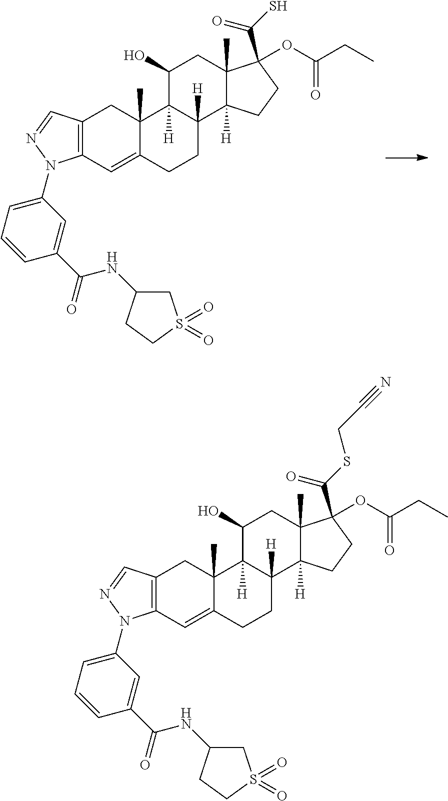

The compound was prepared from intermediate 18 and bromofluoromethane according to the procedure described in Example 11.

APCI-MS m/z: 721 [MH⁺].

¹H NMR (400 MHz, CDCl₃) δ 8.10 (1H, s), 7.85 (1H, dd), 7.69-7.52 (4H, m), 6.16 (1H, s), 4.94 (1H, m), 4.56 (1H, d), 3.79 (1H, d), 3.56 (1H, d), 3.52-3.35 (2H, m), 3.24-2.92 (4H, m), 2.77-2.31 (11H, m), 2.16-1.93 (6H, m), 1.82 (1H, m), 1.65 (1H, m), 1.48 (2H, m), 1.34 (3H, d), 1.27 (1H, m), 1.16 (3H, t), 1.13-1.03 (2H, m), 1.01 (3H, s).

### EXAMPLE 13

### (1R,3aS,3bS,10aR,10bS,11S,12aS)-7-(3-{[(2R)-2-Carbamoylpyrrolidin-1-yl]carbonyl}phenyl)-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate

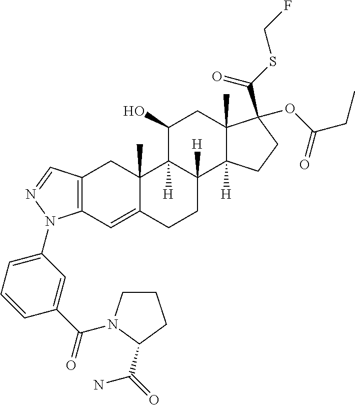

Intermediate 22 (100 mg, 0.17 mmol) was suspended in CH₂Cl₂ (10 ml) and triethylamine (0.115 ml, 0.83 mmol) was added. The mixture was cooled in an ice bath under argon when propionyl chloride (0.064 ml, 0.74 mmol) was added. The mixture was stirred for appr. 10 min and N₁,N₁,N₂-trimethylethane-1,2-diamine (0.042 ml, 0.33 mmol) was added and after 10 min. 110 μl of a 40% solution of bromofluoromethane in DMF was added and the resulting mixture was stirred for 10 min. The solvent was removed in vacuo and the residue was dissolved in CH₂Cl₂ (50 ml). The organic phase was washed with 0.5 M HCl (50 ml), water (50 ml) and brine (50 ml), filtered and evaporated in vacuo. The obtained residue was dissolved in acetonitrile/water (5 ml/1 ml) and the resulting solution was purified using preparative HPLC (MeCN 35%-85%, eluted at 70%, TFA). The product containing fractions were combined and freeze-dried to yield 14 mg (12%) of the desired compound as a solid.

APCI-MS m/z: 693 [MH⁺].

¹H NMR (400 MHz, CDCl₃, TFA-d) δ 7.95-7.93 (1H, m), 7.84 (1H, d), 7.78-7.54 (3H, m), 6.02-5.66 (3H, m), 4.75-4.48 (2H, m), 3.90-3.59 (2H, m), 3.22 (1H, d), 3.07-2.99 (1H, m), 2.85-2.78 (1H, m), 2.63-2.52 (1H, m), 2.50-2.39 (4H, m), 2.27-1.94 (8H, m), 1.92-1.81 (1H, m), 1.73-1.62 (1H, m), 1.56-1.46 (1H, m), 1.40-1.32 (4H, m), 1.24-1.15 (4H, m), 1.00 (3H, s).

### EXAMPLE 14

### (1R,3aS,3bS,10aR,10bS,11S,12aS)-7-(3-{[(2R)-2-Carbamoylpyrrolidin-1-yl]carbonyl}phenyl)-1-{[(cyanomethyl)sulfanyl]carbonyl}-11-hydroxy-10a,12a-dimethyl-1,2,3,3a3b,4,5,7,10,10a,10b,11,12,12a-tetradecahydrocyclopenta[5,6]naphtho[1,2-f]indazol-1-yl propanoate

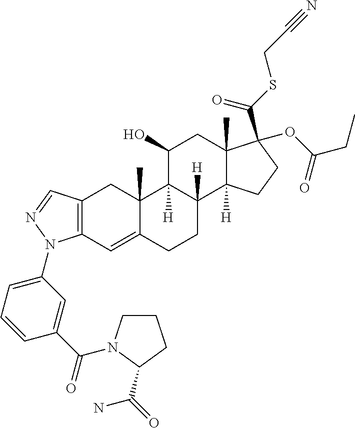

The compound was prepared according to the procedure for example 13, starting from intermediate 22 and 2-bromoacetonitrile.

APCI-MS m/z: 700 [MH⁺].

¹H NMR (400 MHz, CDCl₃, TFA-d) δ 7.95-7.93 (1H, m), 7.83 (1H, d), 7.77-7.53 (3H, m), 6.01-5.98 (1H, m), 4.75-4.48 (2H, m), 3.90-3.59 (4H, m), 3.21 (1H, d), 3.03-2.94 (1H, m), 2.84-2.77 (1H, m), 2.65-2.54 (1H, m), 2.48-2.35 (4H, m), 2.26-1.96 (8H, m), 1.93-1.82 (1H, m), 1.70-1.61 (1H, m), 1.57-1.47 (1H, m), 1.43-1.35 (4H, m), 1.21-1.14 (4H, m), 1.02 (3H, s).

### EXAMPLE 15

### (1R,3aS,3bS,10aS,10bR,11S,12aS)-7-(3-{[(2R)-2-Carbamoylpyrrolidin-1-yl]carbonyl}phenyl)-10b-fluoro-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl methoxyacetate

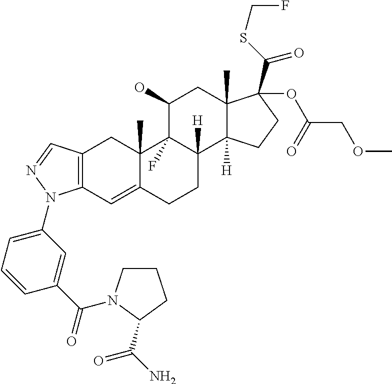

The compound was prepared according to the procedure for example 13, starting from intermediate 26, 2-methoxyacetyl chloride and bromofluoromethane.

APCI-MS m/z: 727 [MH⁺].

¹H NMR (400 MHz, CDCl₃, TFA-d) δ 7.98-7.96 (1H, m), 7.85 (1H, d), 7.78-7.56 (3H, m), 6.08 (1H, s), 5.85 (2H, m), 4.75-4.43 (2H, m), 4.26 (2H, m), 3.90-3.60 (2H, m), 3.57 (3H, s), 3.39 (1H, d), 3.10-2.97 (2H, m), 2.73-2.60 (1H, m), 2.51-1.79 (12H, m), 1.68-1.45 (5H, m), 1.02 (3H, s).

### EXAMPLES 16 TO 18

The compounds of Examples 16 to 18 were prepared by processes analogous to those described in the above Examples or by processes known in the art.

| | Ex. | | | APCI |
|---|---|---|---|---|
| Structure | No. | Compound | ¹H NMR | m/z |
| 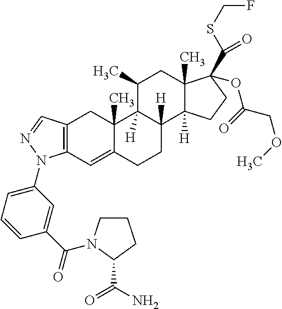 | 16 | (1R,3aS,3bS,10aR,10bS,11S,12aS)-7- (3-{[(2R)-2-carbamoylpyrrolidin-1- yl]carbonyl}phenyl-1- {[(fluoromethyl)sulfanyl]carbonyl}-11- hydroxy-10a,12a-dimethyl- 1,2,3,3a,3b,4,5,7,10,10a,10b,11,12,12a- tetradecahydrocyclopenta[5,6]naphtho [1,2-f]indazol-1-yl methoxyacetate | 1H NMR (400 MHz, CDCl3, TFA-d) δ 7.95 (1H, s), 7.85-7.54 (4H, m), 6.01- 5.69 (3H, m), 4.75-4.49 (2H, m), 4.22 (2H, m), 3.91-3.59 (2H, m), 3.56 (3H, s), 3.19 (1H, d), 3.10-3.01 (1H, m), 2.84-2.77 (1H, m), 2.64-2.50 (1H, m), 2.47-2.38 (2H, m), 2.28-1.98 (8H, m), 1.92-1.82 (1H, m), 1.68-1.59 (1H, m), 1.57-1.46 (1H, m), 1.41-1.30 (4H, m), 1.21-1.10 (1H, m), 1.01 (3H, s). | 709 |
| 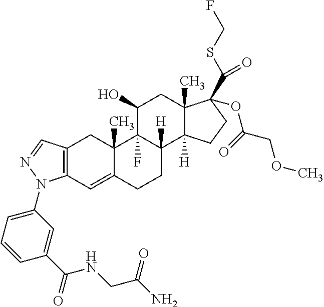 | 17 | (1R,3aS,3bS,10aS,10bR,11S,12aS)-7- {3-[(2-amino-2-oxoethyl)carbamoyl]- phenyl}-10b-fluoro-1- {[(fluoromethyl)sulfanyl]carbonyl}-11- hydroxy-10a,12a-dimethyl- 1,2,3,3a,3b,4,5,7,10,10a,10b,11,12,12a- tetradecahydrocyclopenta[5,6]naphtho [1,2-f]indazol-1-yl methoxyacetate | 1H NMR (400 MHz, DMSO-d6) δ 8.86 (1H, t), 7.99 (1H, s), 7.89 (1H, d), 7.67- 7.59 (1H, m), 7.54 (1H, s), 7.40 (1H, s), 7.04 (1H, s), 6.27 (1H, s), 5.99 (1H, s), 5.86 (1H, s), 5.23 (1H, s), 4.26 (1H, s), 4.18-4.05 (2H, m), 3.82 (2H, d), 3.32 (3H, s), 3.15 (1H, d), 2.86-2.78 (2H, m), 2.38-2.17 (4H, m), 2.10-1.90 (2H, m), 1.84 (1H, d), 1.77-1.64 (2H, m), 1.48- 1.35 (2H, m), 1.33 (3H, s), 0.88 (3H, s). | 687 |
| 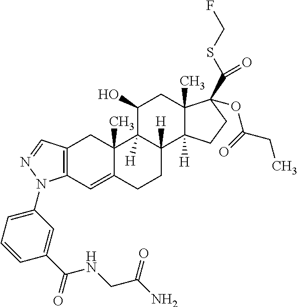 | 18 | (1R,3aS,3bS,10aR,10bS,11S,12aS)-7- {3-[(2-amino-2- oxoethyl)carbamoyl]phenyl}-1- {[(fluoromethyl)sulfanyl]carbonyl}-11- hydroxy-10a,12a-dimethyl- 1,2,3,3a,3b,4,5,7,10,10a,10b,11,12,12a- tetradecahydrocyclopenta[5,6]naphtho [1,2-f]indazol-1-yl propanoate | 1H NMR (400 MHz, DMSO-d6) δ 8.85 (1H, t), 7.99 (1H, s), 7.88 (1H, d), 7.66- 7.59 (2H, m), 7.51 (1H, s), 7.40 (1H, s), 7.04 (1H, s), 6.19 (1H, s), 5.99-5.93 (1H, m), 5.87-5.80 (1H, m), 4.57-4.49 (1H, m), 4.37 (1H, s), 3.82 (2H, d), 2.96 (1H, d), 2.82-2.74 (1H, m), 2.69-2.63 (1H, m), 2.45-2.27 (4H, m), 1.96-1.83 (5H, m), 1.78-1.70 (1H, m), 1.66-1.57 (1H, m), 1.42-1.30 (1H, m), 1.23 (3H, s), 1.15 (1H, d), 1.03 (3H, t), 0.88 (3H, s). | 653 |

### Intermediate 27

### (8S,9R,10S,11S,13S,14S,16R,17R)-9-Fluoro-2-formyl-11,17-dihydroxy-10,13,16-trimethyl-3-oxo-2,3,6,7,8,9,10,11,12,13,14,15,16,17-tetradecahydro-1H-cyclopenta[a]phenanthrene-17-carbothioic S-acid

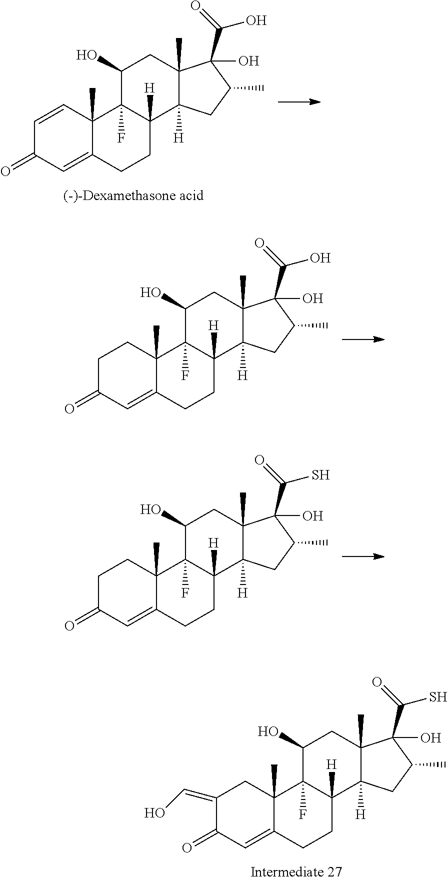

A 2 L flask was charged with (−)-Dexamethasone acid (92.31 g, 243.93 mmol), tris(triphenylphosphine)rhodium(I)chloride (11.28 g, 12.20 mmol), toluene (1000 mL) and ethanol (300 mL) and the mixture was heated at 50° C. under pressure 55 psi for 50 hours. The solvents were removed under pressure and the residue coevaporated with EtOH twice (2×250 mL). Dichloromethane 1.2 L was added and the slurry stirred on the rotavapor overnight. Filtered, washed three times with dichloromethane (3×100 mL) and dried to afford 86.5 g of a solid material. NMR showed some 10% unreacted starting material and approx 1.5 mol % of the catalyst. The 86.5 g of the impure product above was hydrogenated with the same solvent composition and with tris(triphenylphosphine)rhodium(I)chloride (1.5 g, 1.62 mmol) at 50° C. under pressure 55 psi for a further 30 hours. The solvents were removed under pressure and the residue coevaporated with EtOH two times (2×250 mL). Dichloromethane 1.2 L was added and the slurry stirred for one hour before being filtered, washed three times with dichloromethane (3×100 mL) and dried to afford 79.4 g of the di-hydro product. HPLC purity approx. 92%. This material was used in the next step without further purification. APCI-MS M/z: 381.2 [MH+].

The di-hydro product above (79.4 g, 208.70 mmol) was dissolved in DMF (620 mL) in a 5 L 5-neck reactor flask (equipped with overhead stirrer, thermometer and dropping funnel) and di(1H-imidazol-1-yl)methanone (67.7 g, 417.40 mmol) was added in portions. During the last addition, another 100 mL of DMF was used to rinse the vessel and the mixture stirred at room temperature overnight. A gas trap containing sodium hypochlorite was connected to the reactor and H₂S (g) was bubbled for 60 minutes and stirring was continued for a further 60 minutes before adding water (2 L) into the reaction mixture. While keeping the temperature between 25-30° C., 2 N HCl (aq, 600 mL) was added dropwise and the reaction mixture was stirred for 60 minutes. The resulting precipitate was filtered, air dried overnight and for two days in vacuo at 50° C. to give 82.8 g of the thioacid as a white solid. This impure material was used in the next step without further purification. APCI-MS M/z: 397.0 [MH+]

To a stirred suspension of sodium hydride (160 g, 3994.80 mmol) in THF (3000 mL) under argon atmosphere the thio acid from the step above (158.4 g, 399.48 mmol) was added in small portions over a period of 20 minutes, while keeping the temperature below 25° C. The reaction mixture was cooled to 15° C. and ethyl formate (1614 mL, 19973.98 mmol) was added carefully during the addition of the first 2-300 mL. After 5 hours stirring the reaction mixture was quenched by the careful addition of 1M NaOH (1500 mL). The aqueous phase was collected and the organic phase was extracted with an additional 2×750 mL of 1M NaOH. The combined alkaline aqueous phases were washed with 2×1.5 L of TBME. The aqueous phase was acidified to pH 3-4 with 5 NHCL added in portions while cooling. Stirring continued for one hour before filtering, washing with some water and drying. The solid was washed with small amounts of TBME and then dried again in vacuo at 50° C. affording 102.5 g (60%) of the title compound.

APCI-MS M/z: 425.0 [MH+]. ¹H NMR (400.0 MHz, cdcl3) δ 7.96 (1H, s), 7.79 (1H, d), 7.64 (1H, d), 7.54 (1H, t), 7.48 (1H, s), 7.09 (1H, d), 6.23 (1H, d), 6.19 (1H, s), 5.94 (1H, dd), 5.82 (1H, dd), 5.57 (1H, s), 4.74 (1H, p), 4.45 (1H, d), 4.17-4.07 (2H, dd), 3.50-3.39 (4H, m), 3.32 (1H, d), 2.78 (1H, d), 2.58 (1H, ddd), 2.45-2.18 (4H, m), 1.88 (2H, m), 1.75-1.66 (1H, m), 1.54 (3H, d), 1.42-1.33 (4H, m), 1.11 (3H, s), 1.04 (3H, d).

### Intermediate 28

### (8S,9R,10S,11S,13S,14S,16R,17R)-9-Fluoro-2-formyl-11,17-dihydroxy-10,13-dimethyl-3-oxo-2,3,6,7,8,9,10,11,12,13,14,15,16,17-tetradecahydro-1H-cyclopenta[a]phenanthrene-17-carbothioic S-acid

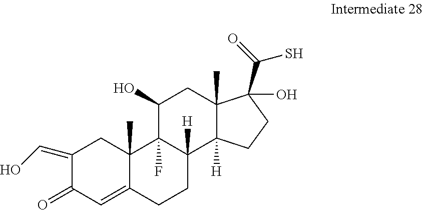

Intermediate 28 was prepared starting from Intermediate 23 using processes analogous to those described for Intermediate 27.

APCI-MS M/z: 411.1 [MH+].

### Intermediate 29

### (8S,9R,10S,11S,13S,14S,16R,17R)-2-Formyl-11,17-dihydroxy-10,13-dimethyl-3-oxo-2,3,6,7,8,9,10,11,12,13,14,15,16,17-tetradecahydro-1H-cyclopenta[a]phenanthrene-17-carbothioic S-acid

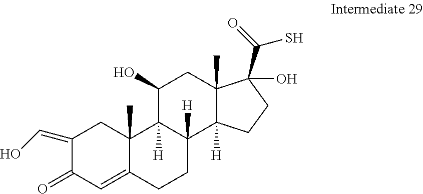

Intermediate 29 was prepared starting from Intermediate 1 using processes analogous to those described for Intermediate 27.

APCI-MS M/z: 393.3[MH+].

### Intermediate 30

### (2Z,6alpha,11beta,17alpha)-11,17-Dihydroxy-2-(hydroxymethylidene)-6-methyl-3-oxoandrost-4-ene-17-carbothioic S-acid

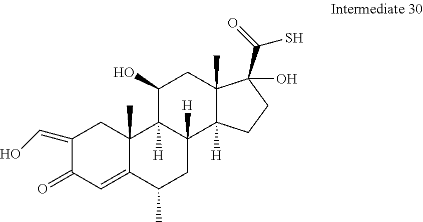

Intermediate 30 was prepared starting from (6alpha,11beta,17alpha)-11,17-dihydroxy-6-methyl-3-oxoandrosta-1,4-diene-17-carboxylic acid using processes analogous to those described for Intermediate 27.

APCI-MS M/z: 407.3 [MH+].

### EXAMPLE 22

### (1R,2R,3aS,3bS,10aS,10bR,11S,12aS)-7-(3-{[(1S)-2-Amino-1-methyl-2-oxoethyl]carbamoyl}phenyl)-10b-fluoro-1-{[(fluoromethyl)sulfanyl]carbonyl}-11-hydroxy-2,10a,12a-trimethyl-1,2,3,3a,3b,4,5,7,10,10a,10b,11,12,12a-tetradecahydrocyclopenta[5,6]naphtho[1,2-f]indazol-1-yl methoxyacetate

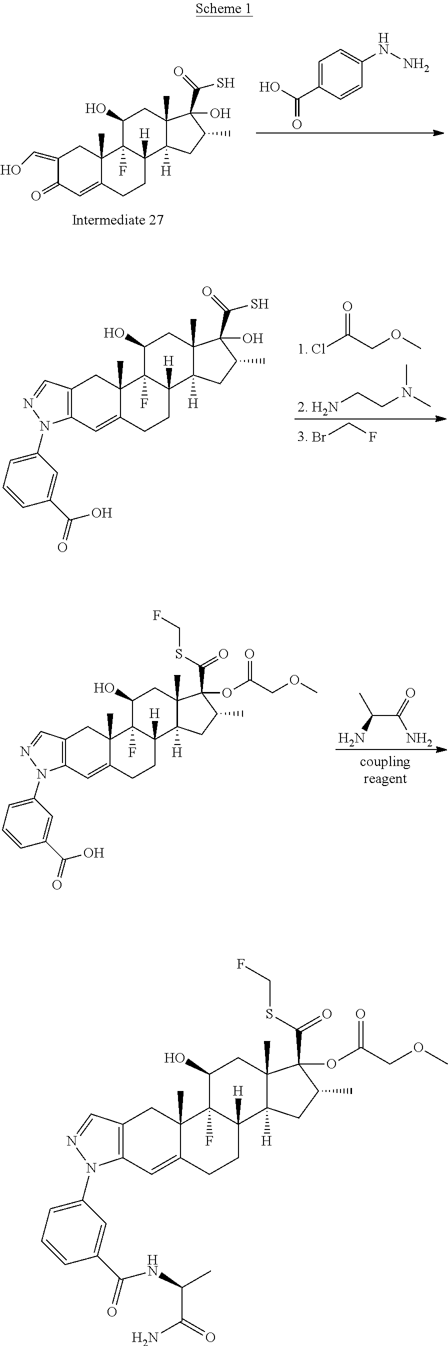 [0425] (i) Intermediate 27 (1.274 g, 3 mmol) and 3-hydrazinylbenzoic acid (0.456 g, 3.00 mmol) were stirred in a mixture of acetic acid (15 mL) and water (3 mL) overnight at room temperature. Then the reaction mixture was poured into water, and the precipitate collected by filtration, and dried. The product obtained (1.128 g; (70% yield) was used in the next step.
(ii) The product from the step (i) above (1.128 g, 2.09 mmol) was dissolved in acetone (30 ml) to give a brown solution, which was cooled to 0° C. To the mixture was added triethylamine (1.157 ml, 8.35 mmol), and a sticky precipitate formed. Then a solution of 2-methoxyacetyl chloride (0.702 g, 6.47 mmol) in acetone (5 ml) was added, and the mixture was stirred for 30 minutes. N1-ethyl-N2,N2-dimethylethane-1,2-diamine (0.688 ml, 4.38 mmol) was added, and the mixture was stirred for another 10 minutes at 0° C. A solution of bromofluoromethane (0.353 g, 3.13 mmol) in DMF (28% wt., 1.26 g) was added, the cooling bath was removed, and the mixture was stirred at room temperature overnight. Then EtOAc (100 ml) was added, and the mixture was washed with aqueous HCl (0.5 M, 2×100 ml). The organic phase was then dried over Na₂SO₄, and was filtered and evaporated to give a brown solid (1.192 g of the crude product, 89% yield) which was used in the next step without further purification.
(iii) To a stirred solution of crude product from step (ii) (200 mg, 0.31 mmol) in NMP (5 ml) was added 2-(1H-benzo[d][1,2,3]triazol-1-yl)-1,1,3,3-tetramethylisouronium tetrafluoroborate (199 mg, 0.88 mmol), followed by (S)-2-aminopropanamide hydrochloride (38.6 mg, 0.31 mmol) and N-ethyl-N-isopropylpropan-2-amine (0.159 ml, 0.93 mmol). The mixture was stirred at room temperature for 1 hour. EtOAc (50 ml) was added, and the organic phase was washed with aqueous 1M NaHCO₃, 0.5 M HCl, and brine. The organic phase was dried with Na₂SO₄, filtered and evaporated. The product was purified by semi-prep HPLC (Kromasil column, methanol/water). The HPLC purification was repeated using MeCN/water as eluant (gradient from 50 to 90%). Yield 37 mg (17%) of the desired compound.

¹H NMR (400.0 MHz, CDCl₃) δ 7.96 (1H, s), 7.79 (1H, d), 7.64 (1H, d), 7.54 (1H, t), 7.48 (1H, s), 7.09 (1H, d), 6.23 (1H, d), 6.19 (1H, s), 5.94 (1H, dd), 5.82 (1H, dd), 5.57 (1H, s), 4.74 (1H, p), 4.45 (1H, d), 4.17-4.07 (2H, dd), 3.50-3.39 (4H, m), 3.32 (1H, d), 2.78 (1H, d), 2.58 (1H, ddd), 2.45-2.18 (4H, m), 1.88 (2H, m), 1.75-1.66 (1H, m), 1.54 (3H, d), 1.42-1.33 (4H, m), 1.11 (3H, s), 1.04 (3H, d).

### EXAMPLES 25-29, 41-43, 45-47

The compounds of Examples 25-29, 41-43, 45-47 were prepared starting from Intermediate 27 using processes analogous to those described in Example 22.

### EXAMPLES 32-36, 44

The compounds of Examples 32-36, 44 were prepared starting from Intermediate 28 using processes analogous to those described in Example 22.

### EXAMPLES 39-40, 48-52

The compounds of Examples 39-40, 48-52 were prepared starting from Intermediate 29 using processes analogous to those described in Example 22.

### EXAMPLES 23-24, 30-31, 37-38

The compounds of Examples 23-24, 30-31, 37-38 were prepared starting from Intermediate 30 using processes analogous to those described in Example 22.

### EXAMPLE 53

### (1R,3aS,3bS,10aS,10bR,11S,12aS)-7-(3-{[(2R)-2-carbamoylpyrrolidin-1-yl]carbonyl}phenyl)-10b-fluoro-11-hydroxy-10a,12a-dimethyl-1-[(methylsulfanyl)carbonyl]-1,2,3,3a,3b,4,5,7,10,10a,10b,11,12,12a-tetradecahydrocyclopenta[5,6]naphtho[1,2-f]indazol-1-yl cyclopropanecarboxylate

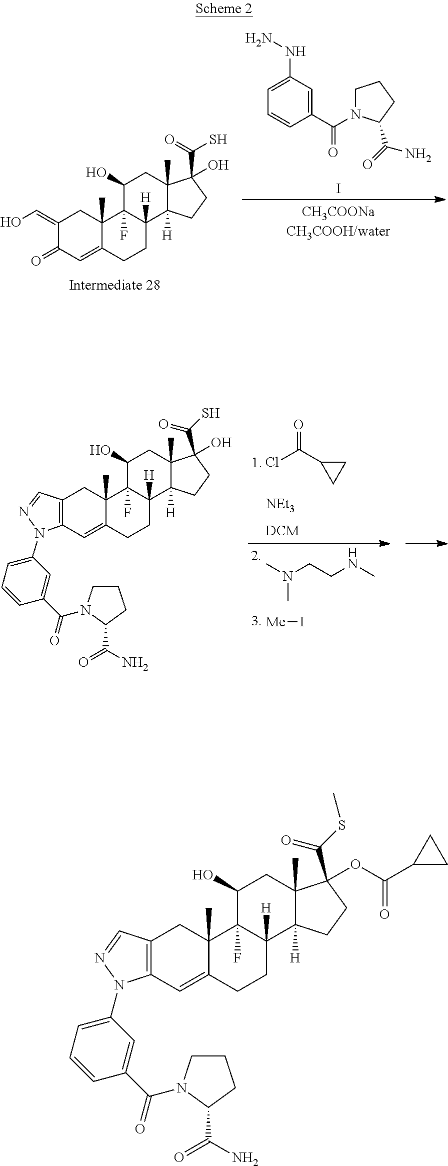 [0434] (i) To a stirred solution of Intermediate 28 (0.747 g, 1.82 mmol) in acetic acid (15 ml) and water (3 ml) was added sodium acetate (0.149 g, 1.82 mmol), followed by (R)-1-(3-hydrazinylbenzoyl)pyrrolidine-2-carboxamide (0.452 g, 1.82 mmol). The mixture was stirred overnight. The mixture was poured into water (100 ml) and the precipitate was collected by filtration and used directly in the next step.
(ii) A stirred solution of the product from the previous step and triethylamine (0.227 ml, 1.64 mmol) in DCM (20 ml) was cooled to 0° C. under argon, and a solution of cyclopropanecarbonyl chloride in DCM (20 ml) was added. The mixture was stirred for 1 hour. N1,N1,N2-trimethylethane-1,2-diamine (0.157 ml, 1.23 mmol) in was added. Stirring was continued at 0° C., and the reaction was monitored by LC-MS, and after the diacetylated product had disappeared (30 minutes), the solution of methyl iodide in DCM (20 ml) was added. The cooling bath was removed, and stirring was continued for 1 hour. The reaction mixture was diluted with DCM (100 ml), transferred into a separation funnel, and washed with HCl (1M, aq, 100 ml). The aqueous layer was then extracted with DCM (100 ml), and the combined organic layers were dried with Na₂SO₄. Eveporation of solvent afforded crude product as brown oil, which solidified (4.273 g crude product). It was dissolved in EtOAc, silica gel was added (about 60 g), and the solvent was removed. The obtained plug was placed on the top of a silica gel column and the product was eluted with n-heptane/EtOAc mixture (1:1). The fractions containing product were collected, and the solvent was removed, affording the yellow solid (1.21 g). It was recrystallized fron MeCN/water mixture, yielding pale-yellow crystalline material, 809 mg.

¹H NMR (400.0 MHz, CDCl₃) δ 7.68 (1H, s), 7.62-7.47 (4H, m), 6.91 (1H, s), 6.24 (1H, d), 5.37 (1H, s), 4.81 (1H, dd), 4.48 (1H, d), 3.66-3.50 (2H, m), 3.35 (1H, d), 3.05-2.95 (1H, m), 2.81 (1H, d), 2.60 (1H, m), 2.49 (2H, m), 2.40-2.29 (5H, m), 2.27-1.58 (11H, m), 1.43 (4H, m), 1.25 (3H, d), 1.14-1.01 (2H, m), 0.94 (3H, s), 0.92 (2H, m). APCI-MS m/z: 705 [MH+].

### EXAMPLES 54-55

The compounds of Examples 54-55 were prepared starting from Intermediate 28 using processes analogous to those described for Example 53.

### EXAMPLE 56

### (1R,3aS,3bS,10aSJObR,11S,12aS)-7-(3-{[(2R)-2-carbamoylpyrrolidin-1-yl]carbonyl}phenyl)-10b-fluoro-11-hydroxy-10a,12a-dimethyl-1-[(methylsulfanyl)carbonyl]-1,2,3,3a,3b,4,5,7,10,10a,10b,11,12,12a-tetradecahydrocyclopenta[5,6]naphtho[1,2-f]indazol-1-yl methoxyacetate

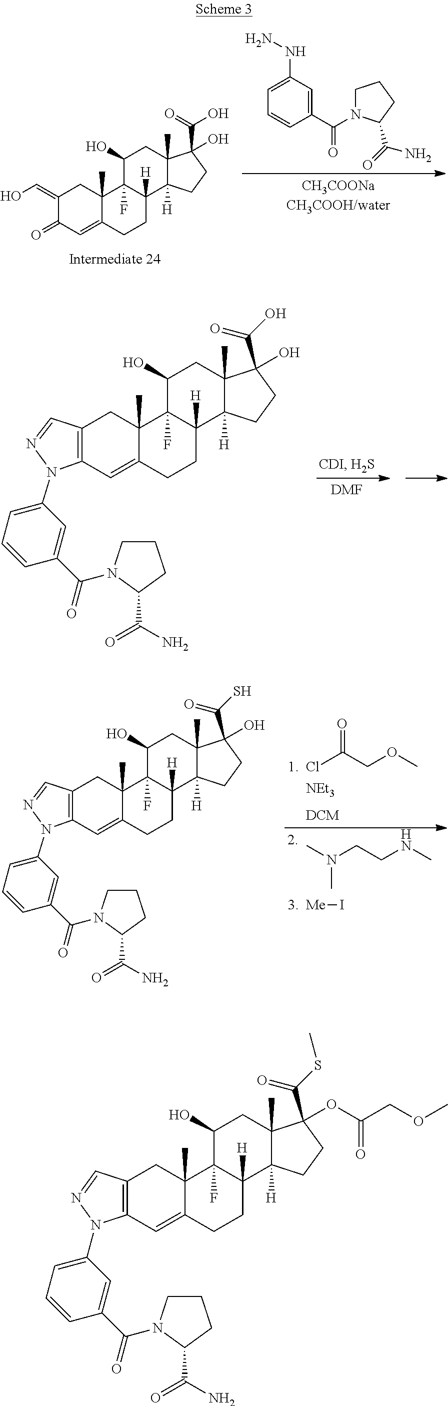 [0439] (i) To a stirred solution of Intermediate 24 (3.38 g, 8.57 mmol) in acetic acid (30 ml) and water (6 mL) was added (R)-1-(3-hydrazinylbenzoyl)pyrrolidine-2-carboxamide (prepared analogously as for Intermediate 3, 3.72 g, 10.28 mmol) and potassium acetate (1.682 g, 17.13 mmol). The mixture was poured into water (200 ml) and the precipitate was filtered off. 4.828 g of solid was retrieved. It was used in the next step without further purification.
(ii) The product obtained in step (i) above (971 mg, 1.60 mmol) was dissolved in DMF (10 mL), and di(1H-imidazol-1-yl)methanone (649 mg, 4.00 mmol) was added. The mixture was stirred at room temperature for 3 hours. Hydrogen sulfide (1.60 mmol) was bubbled through the stirred solution for 5 min, then the stirring was continued at room temperature for 10 minutes in a sealed flask. The mixture was poured into a mixture of ice (150 ml) and aq. HCl (20 ml, 2 M). After the ice had melted the mixture was extracted with EtOAc (2×50 ml); some insoluble material which had formed was removed. The organic layer was dried with Na₂SO₄, and the solvent was removed in vacuo to give a dark-yellow oil which was used in the next step without further purification.
(iii) A stirred solution of the product from the previous step and triethylamine (0.134 ml, 0.96 mmol) in DCM (20 ml) was cooled to 0° C. under argon, and a solution of methoxyacetyl chloride in DCM (20 ml) was added. The mixture was stirred for 1 hour. N1,N1,N2-trimethylethane-1,2-diamine (0.092 ml, 0.72 mmol) was added. Stirring was continued at 0° C., and the reaction was monitored by LC-MS. After the diacetylated product had disappeared (30 minutes), the solution of methyl iodide in DCM (20 ml) was added. The cooling bath was removed, and stirring was continued for 1 hour. The reaction mixture was diluted with DCM (100 ml), transferred into a separation funnel, and washed with HCl (1M, aq, 100 ml). The aqueous layer was then extracted with DCM (100 ml), and the combined organic layers were dried with Na₂SO₄. Evaporation of solvent afforded crude product as brown oil, which solidified (4.273 g crude product). It was dissolved in EtOAc, silica gel was added (about 60 g), and the solvent was removed. The obtained plug was placed on the top of a silica gel column, and the product was eluted with n-heptane/EtOAc mixture (1:1). The fractions containing product were collected, and the solvent was removed, affording a yellow solid (1.21 g). It was recrystallized from MeCN/water mixture, yielding pale-yellow crystalline material, 809 mg.

¹H NMR (400.0 MHz, CDCl₃) δ 7.68 (1H, s), 7.53 (4H, ddd), 6.92 (1H, s), 6.23 (1H, d), 5.42 (1H, s), 4.80 (1H, dd), 4.46 (1H, d), 4.07 (2H, dd), 3.67-3.50 (2H, m), 3.47 (3H, s), 3.33 (1H, d), 3.11-3.01 (1H, m), 2.79 (1H, d), 2.61 (1H, m), 2.48 (2H, m), 2.37 (1H, m), 2.33 (3H, s), 2.30-2.02 (6H, m), 1.93-1.43

### EXAMPLES 57-62

The compounds of Examples 57-62were prepared starting from Intermediate 24 using processes analogous to those described in Example 56.

| | Ex. | | | APCI |
|---|---|---|---|---|
| Structure | No. | Compound | ¹H NMR | m/z |
| 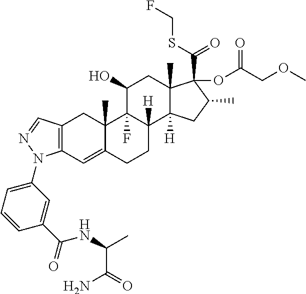 | 22 | (1R,2R,3aS,3bS,10aS,10bR,11S,12aS)- 7-(3-{[(1S)-1-amino-1-methyl-2- oxoethyl]carbamoyl}phenyl)-10b- fluoro-1-{[(fluoromethyl)sulfanyl]- carbonyl}-11-hydroxy-2,10a,12a- trimethyl-1,2,3,3a,3b,4,5,7,10,10a- 10b,11,12,12a-tetradecahydro- cyclopenta[5,6]naphtho[1,2-f]indazol- 1-yl methoxyacetate | 1H NMR (400.0 MHz, CDCl3) δ 7.96 (1H, s), 7.79 (1H, d), 7.64 (1H, d), 7.54 (1H, t), 7.48 (1H, s), 7.09 (1H, d), 6.23 (1H, d), 6.19 (1H, s), 5.94 (1H, dd), 5.82 (1H, dd), 5.57 (1H, s), 4.74 (1H, p), 4.45 (1H, d), 4.17- 4.07 (2H, dd), 3.50-3.39 (4H, m), 3.32 (1H, d), 2.78 (1H, d), 2.58 (1H, ddd), 2.45-2.18 (4H, m), 1.88 (2H, m), 1.75- 1.66 (1H, m), 1.54 (3H, d), 1.42-1.33 (4H, m), 1.11 (3H, s), 1.04 (3H, d). | 715 |
| 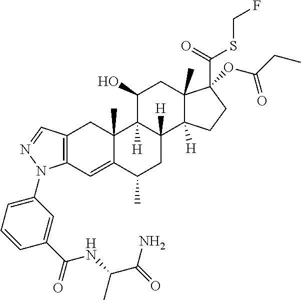 | 23 | (1R,3aS,3bS,5S,10aR,10bS,11S,12aS)- 7-(3-{[(1S)-2-amino-1-methyl-2- oxoethyl]carbamoyl}phenyl)-1- {[(fluoromethyl)sulfanyl]carbonyl}-11- hydroxy-5,10a-12a-trimethyl- 1,2,3,3a,3b,4,5,7,10,10a,10b,11,12,12a- tetradecahydrocyclopenta[5,6]naphtho [1,2-f]indazol-1-yl propanoate | 1H NMR (499.875 MHz, DMSO) δ: 8.61 (1H, d), 8.03 (1H, s), 7.91 (1H, d), 7.56-7.70 (3H, m), 7.52 (1H, s), 7.38 (1H, s), 6.99 (1H, s), 6.15 (1H, s), 5.94 (1H, m), 5.84 (1H, m), 4.53 (1H, d), 4.43 (2H, quintet), 4.37 (1H, s), 2.94 (1H, d), 2.70 (2H, d), 2.35 (2H, m), 1.96 (3H, m), 1.73 (1H, dd), 1.59 (1H, m), 1.36 (5H, m), 1.26 (3H, s), 1.13 (1H, dd), 1.04 (6H, m), 0.90 (3H, d), 0.78 (1H, q). | 681.3 |
| 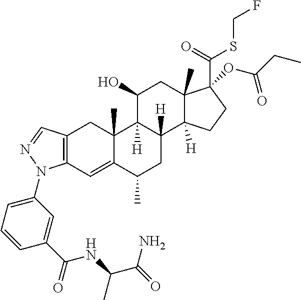 | 24 | (1R,3aS,3bS,5S,10aR,10bs,11S,12aS)- 7-(3-{[(1R)-2-amino-1-methyl-2- oxoethyl]carbamoyl}phenyl)-1- {[(fluoromethyl)sulfanyl]carbonyl}-11- hydroxy-5,10a,12a-trimethyl- 1,2,3,3a,3b,4,5,7,10,10a,10b,11,12,12a- tetradecahydrocyclopenta[5,6]naphtho [1,2-f]indazol-1-yl propanoate | 1H NMR (499.875 MHz, DMSO) δ: 8.62 (1H, d), 8.03 (1H, s), 7.91 (1H, d), 7.65 (3H, m), 7.52 (1H, s), 7.38 (1H, s), 6.98 (1H, s), 6.15 (1H, s), 5.94 (1H, dd), 5.84 (1H, dd), 4.53 (1H, d), 4.43 (1H, m), 4.37 (1H, s), 2.94 (1H, d), 2.72 (2H, m), 2.34 (2H, m), 1.92 (3H, m), 1.73 (1H, d), 1.58 (1H, m), 1.36 (5H, dd), 1.26 (3H, s), 1.13 (1H, d), 1.04 (6H, m), 0.88 (3H, s), 0.78 (1H, q). | 681.3 |
| 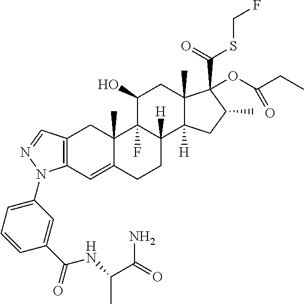 | 25 | (1R,2R,3aS,3bS,10aS,10bR,11S,12aS)- 7-(3-{[(1S)-2-amino-1-methyl-2- oxoethyl]carbamoyl}phenyl)-10b- fluoro-1-{[(fluoromethyl)- sulfanyl]carbonyl}-11-hydroxy- 2,10a,12a-trimethyl- 1,2,3,3a,3b,4,5,7,10,10a,10b,11,12,12a- tetradecahydrocyclopenta[5,6]naphtho [1,2-f]indazol-1-yl propanoate | 1H NMR (399.99 MHz, DMSO) δ: 8.64 (1H, d), 8.09 (1H, s), 7.91 (1H, d), 7.62 (2H, s), 7.62 (2H, s), 7.53 (1H, s), 7.42 (1H, s), 7.01 (1H, s), 6.29 (1H, s), 6.00 (1H, m), 5.86 (1H, m), 5.22 (1H, m), 4.43 (1H, m), 4.24 (1H, m), 4.08 (2H, q), 3.17 (3H, d), 2.79 (1H, d), 2.26 (4H, m), 1.86 (2H, m), 1.62 (1H, m), 1.37 (3H, m), 1.25 (3H, t), 0.96 (3H, s), 0.90 (3H, d) | 699.3 |
| 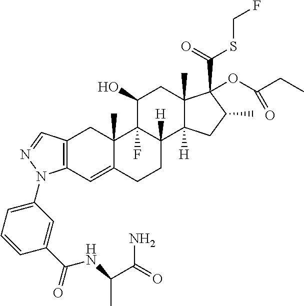 | 26 | (1R,2R,3aS,3bS,10aS,10bR,11S,12aS)- 7-(3-{[(1R)-2-amino-1-methyl-2- oxoethyl]carbamoyl}phenyl)-10b- fluoro-1-{[(fluoromethyl)- sulfanyl]carbonyl}-11-hydroxy- 2,10a,12a-trimethyl- 1,2,3,3a,3b,4,5,7,10,10a,10b,11,12,12a- tetradecahydrocyclopenta[5,6]naphtho [1,2-f]indazol-1-yl propanoate | 1H NMR (399.99 MHz, DMSO) δ: 8.63 (1H, d), 7.97 (1H, s), 7.91 (1H, d), 7.60 (2H, m), 7.48 (1H, s), 7.48 (1H, s), 7.04 (1H, s), 6.34 (1H, s), 6.00 (1H, m), 5.88 (1H, m), 5.23 (1H, m), 4.43 (1H, m), 4.24 (1H, dd), 2.58 (1H, m), 2.37 (5H, m), 2.13 (4H, m), 1.86 (4H, m), 1.63 (1H, m), 1.41 (1H, m), 1.33 (4H, m), 1.26 (1H, t), 1.04 (3H, t), 1.01 (3H, s), 0.91 (3H, d). | 699.3 |
| 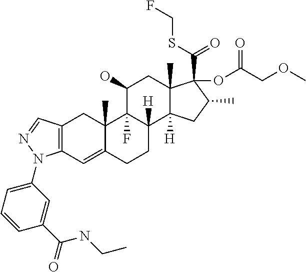 | 27 | (1R,2R,3aS,3bS,10aS,10bR,11S,12aS)- 7-[3-(ethylcarbamoyl)phenyl]-10b- fluoro-1-{[(fluoromethyl)sulfanyl]- carbonyl}-11-hydroxy-2,10a,12a- trimethyl-1,2,3,3a,3b,4,5,7,10,10a, 10b,11,12,12a- tetradecahydrocyclopenta[5,6]naphtho [1,2-f]indazol-1-yl methoxyacetate | 1H NMR (400.0 MHz, CDCl3) δ 7.88 (1H, t), 7.79-7.74 (1H, m), 7.61 (1H, d), 7.53 (1H, t), 7.48 (1H, s), 6.28 (1H, s), 6.25 (1H, d), 5.94 (1H, dd), 5.82 (1H, dd), 4.45 (1H, d), 4.12 (2H, d), 3.58- 3.47 (2H, m), 3.45 (4H, m), 3.32 (1H, d), 2.79 (1H, d), 2.67-2.52 (1H, m), 2.46-2.19 (4H, m), 1.89 (2H, m), 1.73-1.65 (1H, m), 1.38 (4H, m, 1.27 (3H, t), 1.11 (3H, s), 1.04 (3H, d). | 672 |
| 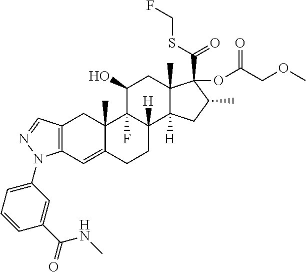 | 28 | 1R,2R,3aS,3bS,10aS,10bR,11S,12aS)- 10b-fluoro-1- {[(fluoromethyl)sulfanyl]carbonyl}-11- hydroxy-2,10a,12a-trimethyl-7-[3- (methylcarbamoyl)phenyl]- 1,2,3,3a,3b,4,5,7,10,10a,10b,11,12,12a- tetradecahydrocyclopenta[5,6]naphtho [1,2-f]indazol-1-yl methoxyacetate | 1H NMR (400.0 MHz, CDCl3) δ 7.87 (1H, t), 7.75 (1H, d), 7.63 (1H, d), 7.53 (1H, t), 7.47 (1H, s), 6.35 (1H, d), 6.26-6.22 (1H, m), 5.94 (1H, dd), 5.82 (1H, dd), 4.44 (1H, d), 4.11 (2H, dd), 3.49-3.39 (4H, m), 3.32 (1H, d), 3.03 (3H, d), 2.79 (1H, d), 2.59 (1H, td), 2.47- 2.17 (4H, m), 1.94-1.85 (2H, m), 1.42-1.33 (4H, m), 1.11 (3H, s), 1.06-1.02 (3H, m). | 658 |
| 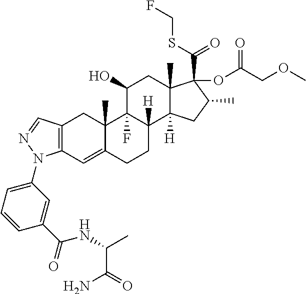 | 29 | (1R,2R,3aS,3bS,10aS,10bR,11S,12aS)- 7-(3-{[(1R)-2-amino-1-methyl-2- oxoethyl]carbamoyl}phenyl)-10b- fluoro-1-{[(fluoromethyl)- sulfanyl]carbonyl}-11-hydroxy- 2,10a,12a-trimethyl- 1,2,3,3a,3b,4,5,7,10,10a,10b,11,12,12a- tetradecahydrocyclopenta[5,6]naphtho [1,2-f]indazol-1-yl methoxyacetate | 1H NMR (400.0 MHz, CDCl3) δ 7.94 (1H, s), 7.78 (1H, d), 7.62 (1H, d), 7.53 (1H, t), 7.46 (1H, s), 7.23 (1H, d), 6.26 (1H, s), 6.21 (1H, d), 5.94 (1H, dd), 5.82 (1H, dd), 5.61 (1H, s), 4.73 (1H, p), 4.44 (1H, d), 4.17-4.06 (2H, dd), 3.49-3.39 (4H, m), 3.32 (1H, d), 2.77 (1H, d), 2.64-2.52 (1H, m), 2.45-2.18 (4H, m), 1.95- 1.81 (2H, m), 1.54 (3H, d), 1.34-1.41 (4H, m), 1.11 (3H, s), 1.04 (3H, d). | 715 |
| 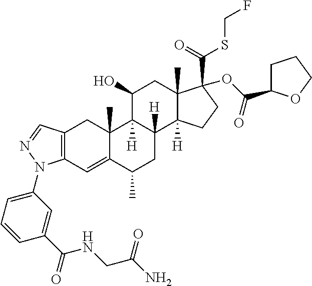 | 30 | (1R,3aS,3bS,5S,10aR,10bS,11S,12aS)- 7-{3-[(2-amino-2- oxoethyl)carbamoyl]phenyl}-1- {[(fluoromethyl)sulfanyl]carbonyl}-11- hydroxy-5,10a,12a-trimethyl- 1,2,3,3a,3b,4,5,7,10,10a,10b,11,12,12a- tetradecahydrocyclopenta[5,6]naphtho [1,2-f]indazol-1-yl (2R)- tetrahydrofuran-2-carboxylate | 1H NMR (399.99 MHz, DMSO) δ: 8.85 (1H, m), 8.15 (1H, s), 7.87 (1H, d), 7.66 (2H, m), 7.54 (1H, s), 7.46 (1H, s), 7.26 (1H, s), 7.16 (1H, s), 7.03 (1H, m), 6.96 (1H, s), 6.11 (1H, s), 5.97 (1H, m), 5.83 (1H, m), 4.60 (1H, m), 4.48 (1H, m), 4.42 (1H, s), 3.82 (4H, d), 2.79 (2H, m), 2.67 (3H, ml), 1.95 (9H, m), 1.59 (1H, m), 1.37 (1H, m), 1.24 (3H, s), 1.14 (1H, m), 1.03 (3H, d), 0.87 (3H, m), 0.79 (1H, m). | 709.2 |
| 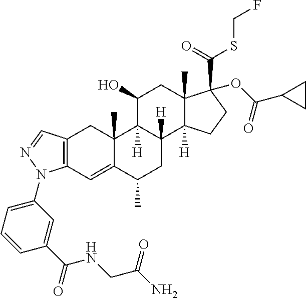 | 31 | (1R,3aS,3bS,5S,10aR,10bS,11S,12aS)- 7-{3-[(2-amino-2- oxoethyl)carbamoyl]phenyl}-1- {[(fluoromethyl)sulfanyl]carbonyl}-11- hydroxy-5,10a,12a-trimethyl- 1,2,3,3a,3b,4,5,7,10,10a,10b,11,12,12a- tetradecahydrocyclopenta[5,6]naphtho [1,2-f]indazol-1-yl cyclopropanecarboxylate | 1H NMR (499.875 MHz, DMSO) δ: 8.85 (1H, t), 8.01 (1H, s), 7.52 (1H, s), 7.39 (1H, s), 7.04 (1H, s), 6.15 (1H, d), 5.94 (1H, d), 5.84 (1H, d), 4.54 (1H, d), 4.37 (1H, d), 3.82 (2H, d), 2.95 (1H, d), 2.63 (1H, t), 2.36 (1H, t), 2.07 (1H, s), 1.85 (8H, m), 1.56 (1H, m), 1.37 (1H, dd), 1.26 (3H, s), 1.15 (2H, m), 1.06 (3H, d), 0.86 (9H, m). | 679.3 |
| 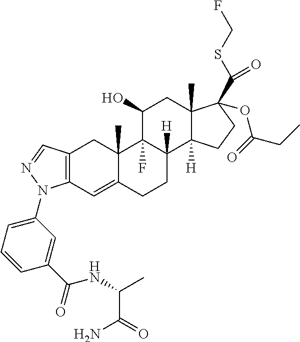 | 32 | (1R,3aS,3bS,10aS,10bR,11S,12aS)-7- (3-{[(1R)-2-amino-1-methyl-2- oxoethyl]carbamoyl}phenyl)-10b- fluoro-1-{[(fluoromethyl)- sulfanyl]carbonyl}-11-hydroxy- 10a,12a-dimethyl- 1,2,3,3a,3b,4,5,7,10,10a,10b,11,12,12a- tetradecahydrocyclopenta[5,6]naphtho [1,2-f]indazol-1-yl propanoate | 1H NMR (400 MHz, CDCl3) δ 7.99 (1H, s), 7.79 (1H, d), 7.63 (1H, d), 7.55 (1H, t), 7.47 (1H, s), 7.10 (1H, d), 6.25 (1H, s), 6.23 (1H, d), 5.99 (1H, dd), 5.70 (1H, dd), 5.66 (1H, s), 4.74 (1H, quintet), 4.47 (1H, d), 3.32 (1H, d), 3.01 (1H, dd), 2.79 (1H, d), 2.66-2.46 (2H, m), 2.41 (2H, q), 2.39-2.14 (3H, m), 2.01 (1H, m), 1.89 (1H, d), 1.83-1.70 (2H, m), 1.60-1.41 (6H, m), 1.40 (3H, s), 1.17 (3H, t), 0.99 (3H, s). | 685 |
| 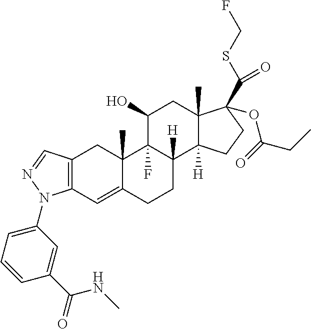 | 33 | (1R,3aS,3bS,10aS,10bR,11S,12aS)- 10b-fluoro-1- {[(fluoromethyl)sulfanyl]carbonyl}-11- hydroxy-10a,12a-dimethyl-7-[3- (methylcarbamoyl)phenyl]- 1,2,3,3a,3b,4,5,7,10,10a,10b,11,12,12a- tetradecahydrocyclopenta[5,6]naphtho [1,2-f]indazol-1-yl propanoate | 1H NMR (400 MHz, CDCl3) δ 7.89 (1H, t), 7.76 (1H, d), 7.63 (1H, d), 7.54 (1H, t), 7.48 (1H, s), 6.27 (2H, m), 5.99 (1H, dd), 5.70 (1H, dd), 4.47 (1H, d), 3.34 (1H, d), 3.03 (3H, d), 3.01 (1H, m), 2.80 (1H, d), 2.66-2.48 (2H, m), 2.41 (3H, q), 2.39-2.14 (2H, m), 2.01 (1H, ddd), 1.89 (1H, d), 1.84-1.71 (2H, m), 1.58-1.43 (2H, m), 1.41 (3H, s), 1.17 (3H, t), 0.99 (3H, s). | 628 |
| 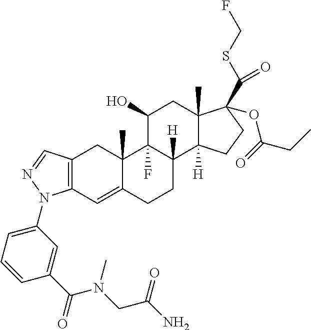 | 34 | (1R,3aS,3bS,10aS,10bR,11S,12aS)-7- {3-[(2-amino-2- oxoethyl)(methyl)carbamoyl]phenyl}- 10b-fluoro-1- {[(fluoromethyl)sulfanyl]carbonyl}-11- hydroxy-10a,12a-dimethyl- 1,2,3,3a,3b,4,5,7,10,10a,10b,11,12,12a- tetradecahydrocyclopenta[5,6]naphtho [1,2-f]indazol-1-yl propanoate | 1H NMR (400 MHz, CDCl3) δ 7.66-7.51 (3H, m), 7.48 (1H, s), 7.45 (1H, d), 6.38 (1H, brs), 6.24 (1H, s), 5.99 (1H, dd), 5.70 (1H, dd), 5.43 (1H, br. s), 4.47 (1H, d), 4.18 (1H, s), 3.33 (1H, d), 3.16 (3H, s), 3.02 (1H, dd), 2.80 (1H, d), 2.66-2.47 (2H, m), 2.41 (2H, q), 2.39-2.14 (3H, m), 2.01 (1H, m), 1.89 (1H, d), 1.77 (2H, m), 1.56-1.42 (2H, m), 1.40 (3H, s), 1.17 (3H, t), 0.99 (3H, s). | 685 |
| 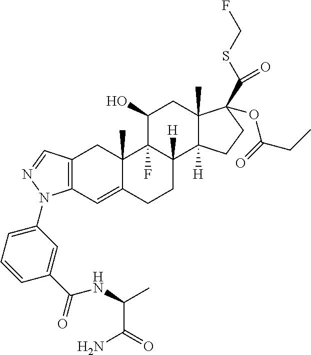 | 35 | (1R,3aS,3bS,10aS,10bR,11S,12aS)-7- (3-{[(1S)-2-amino-1-methyl-2- oxoethyl]carbamoyl}phenyl)-10b- fluoro-1- {[(fluoromethyl)sulfanyl]carbonyl}-11- hydroxy-10a,12a-dimethyl- 1,2,3,3a,3b,4,5,7,10,10a,10b,11,12,12a- tetradecahydrocyclopenta[5,6]naphtho [1,2-f]indazol-1-yl propanoate | 1H NMR (400 MHz, CDCl3) δ 8.00 (1H, s), 7.80 (1H, d), 7.63 (1H, d), 7.54 (1H, t), 7.47 (1H, s), 7.15 (1H, d), 6.28 (1H, s), 6.23 (1H, d), 5.99 (1H, dd), 5.69 (2H, dd), 5.74 (2H, s), 4.75 (1H, quintet), 4.46 (1H, d), 3.32 (1H, d), 3.01 (1H, dd), 2.79 (1H, d), 2.65-2.47 (2H, m), 2.41 (2H, q), 2.39-2.14 (3H, m), 2.00 (1H, m), 1.89 (1H, d), 1.76 (2H, m), 1.59 (1H, m), 1.53 (3H, d), 1.46 (1H, m), 1.40 (3H, s), 1.17 (3H, t), 0.98 (3H, s). | 685 |
| 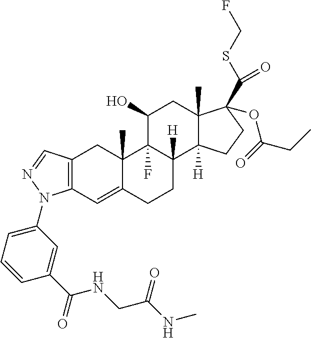 | 36 | (1R,3aS,3bS,10aS,10bR,11S,12aS)- 10b-fluoro-1- {[(fluoromethyl)sulfanyl]carbonyl}-11- hydroxy-10a,12a-dimethyl-7-(3-{[2- (methylamino)-2- oxoethyl]carbamoyl}phenyl)- 1,2,3,3a,3b,4,5,7,10,10a,10b,11,12,12a- tetradecahydrocyclopenta[5,6]naphtho [1,2-f]indazol-1-yl propanoate | 1H NMR (400 MHz, CDCl3) δ 7.99 (1H, s), 7.80 (1H, d), 7.66 (1H, d), 7.55 (1H, t), 7.47 (1H, s), 7.25 (1H, s), 6.22 (2H, m), 5.99 (1H, dd), 5.70 (1H, dd), 4.47 (1H, d), 4.12 (2H, d), 3.33 (1H, d), 3.01 (1H, dd), 2.86 (3H, d), 2.79 (1H, d), 2.65-2.47 (2H, m), 2.41 (2H, q), 2.38-2.13 (3H, m), 2.01 (1H, m), 1.89 (1H, d), 1.83-1.43 (4H, m), 1.40 (3H, s), 1.17 (3H, t), 0.98 (3H, s). | 685 |
| 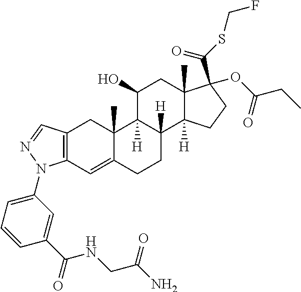 | 37 | (1R,3aS,3bS,5S,10aR,10bS,11S,12aS)- 7-{3-[(2-amino-2- oxoethyl)carbamoyl]phenyl}-1- {[(fluoromethyl)sulfanyl]carbonyl}-11- hydroxy-5,10a,12a-trimethyl- 1,2,3,3a,3b,4,5,7,10,10a,10b,11,12,12a- tetradecahydrocyclopenta[5,6]naphtho [1,2-f]indazol-1-yl propanoate | 1H NMR (499.875 MHz, DMSO) δ: 8.85 (1H, t), 8.01 (1H, s), 7.90 (1H, d), 7.65 (2H, m), 7.52 (1H, s), 7.39 (1H, s), 7.04 (1H, s), 6.15 (1H, d), 5.94 (1H, dd), 5.84 (1H, dd), 4.53 (1H, d), 4.37 (1H, d), 3.82 (2H, d), 2.94 (1H, d), 2.63 (3H, t), 2.33 (1H, m), 1.94 (6H, m), 1.73 (1H, d), 1.58 (1H, td), 1.37 (1H, dd), 1.26 (3H, s), 1.13 (7H, dd), 0.88 (3H, s), 0.78 (1H, q). | 667.3 |
| 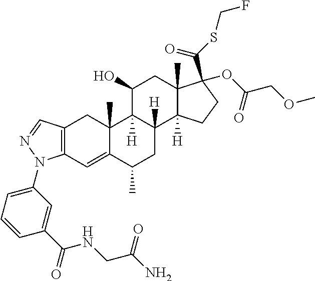 | 38 | (1R,3aS,3bS,5S,10aR,10bS,11S,12aS)- 7-{3-[(2-amino-2- oxoethyl)carbamoyl]phenyl}-1- {[(fluoromethyl)sulfanyl]carbonyl}-11- hydroxy-5,10a,12a-trimethyl- 1,2,3,3a,3b,4,5,7,10,10a,10b,11,12,12a- tetradecahydrocyclopenta[5,6]naphtho [1,2-f]indazol-1-yl methoxyacetate | 1H NMR (499.875 MHz, DMSO) δ: 8.85 (1H, s), 8.01 (1H, s), 7.90 (1H, d), 7.64 (2H, m), 7.52 (1H, s), 7.39 (1H, s), 7.04 (1H, s), 6.15 (1H, s), 5.97 (1H, s), 5.87 (1H, s), 4.55 (1H, d), 4.41 (1H, s), 4.36 (1H, s), 4.13 (1H, d), 4.05 (1H, d), 3.82 (2H, d), 2.94 (1H, d), 2.80 (1H, m), 2.70 (1H, d), 1.96 (7H, m), 1.73 (2H, m), 1.57 (1H, m), 1.38 (1H, m), 1.26 (3H, s), 1.05 (4H, m), 0.89 (3H, s), 0.77 (1H, m). | 683.3 |
| 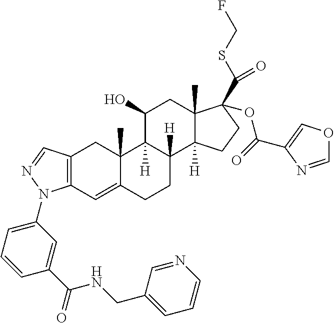 | 39 | (1R,3aS,3bS,10aR,10bS,11S,12aS)-1- {[(fluoromethyl)sulfanyl]carbonyl}-11- hydroxy-10a,12a-dimethyl-7-{3- [(pyridin-3-ylmethyl)carbamoyl]- phenyl}-1,2,3,3a,3b,4,5,7,10,10a,10b, 11,12,12a-tetradecahydrocyclopenta [5,6]naphtho[1,2-f]indazol-1-yl 1,3- oxazole-4-carboxylate | 1H NMR (499.875 MHz, DMSO) δ: 9.28 (1H, s), 9.09 (1H, s), 8.64 (1H, s), 8.58 (2H, s), 8.00 (1H, s), 7.89 (2H, d), 7.69-7.57 (2H, m), 7.53 (1H, s), 6.18 (1H, s), 5.14 (2H, d), 4.55 (1H, d), 4.40 (3H, s), 2.99 (2H, d), 2.86 (1H, m), 2.73 (1H, s), 2.43 (1H, m), 2.30 (1H, m), 1.98 (5H, m), 1.73 (2H, m), 1.39 (2H, m), 1.24 (3H, s), 1.05 (1H, m), 0.94 (3H, s). | 726.2 |
| 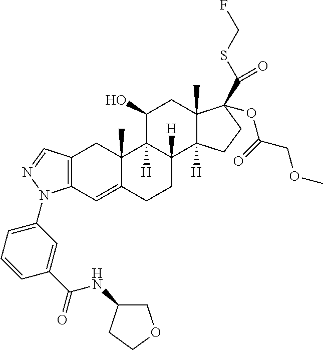 | 40 | (1R,3aS,3bS,10aR,10bS,11S,12aS)-1- {[(fluoromethyl)sulfanyl]carbonyl}-11- hydroxy-10a,12a-dimethyl-7-{3-[(3R)- tetrahydrofuran-3- ylcarbamoyl]phenyl}- 1,2,3,3a,3b,4,5,7,10,10a,10b,11,12,12a- tetradecahydrocyclopenta[5,6]naphtho [1,2-f]indazol-1-yl methoxyacetate | 1H NMR (499.875 MHz, DMSO) δ: 8.70 (1H, d), 7.97 (1H, s), 7.87 (1H, dt), 7.61 (1H, m), 7.51 (1H, s), 6.16 (1H, s), 5.97 (1H, d), 5.87 (1H, d), 4.61 (1H, s), 4.47 (1H, m), 4.36 (1H, d), 4.15 (1H, d), 4.06 (1H, d), 3.86 (2H, m), 3.72 (2H, td), 3.59 (1H, dd), 2.96 (1H, d), 2.80 (1H, dd), 2.65 (2H, d), 2.43 (1H, m), 2.36 (1H, t), 2.31 (1H, s), 2.16 (1H, dd), 1.91 (6H, m), 1.75 (1H, d), 1.61 (1H, d), 1.39 (1H, m), 1.24 (4H, t), 1.13 (1H, dd), 1.01 (1H, m), 0.88 (3H, s). | 682.3 |
| 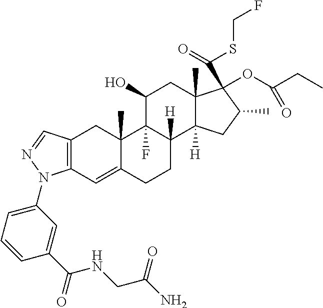 | 41 | (1R,2R,3aS,3bS,10aS,10bR,11S,12aS)- 7-{3-[(2-amino-2- oxoethyl)carbamoyl]phenyl}-10b- fluoro-1-{[(fluoromethyl) sulfanyl]carbonyl}-11- hydroxy-2,10a,12a-trimethyl- 1,2,3,3a,3b,4,5,7,10,10a,10b,11,12,12a- tetradecahydrocyclopenta[5,6]naphtho [1,2-f]indazol-1-yl propanoate | 1H NMR (499.875 MHz, DMSO) δ: 8.86 (1H, t), 7.98 (1H, s), 7.89 (1H, d), 7.53 (1H, s), 7.40 (1H, s), 7.04 (1H, s), 6.26 (1H, s), 5.98 (1H, s), 5.88 (1H, s), 5.22 (1H, d), 4.24 (1H, s), 3.82 (2H, d), 3.15 (1H, d), 2.81 (1H, d), 2.63 (1H, t), 2.26 (8H, m), 1.86 (2H, dd), 1.63 (1H, t), 1.40 (1H, dd), 1.32 (3H, s), 1.25 (1H, m), 1.06 (4H, m), 1.01 (3H, s), 0.90 (3H, d). | 685.3 |
| 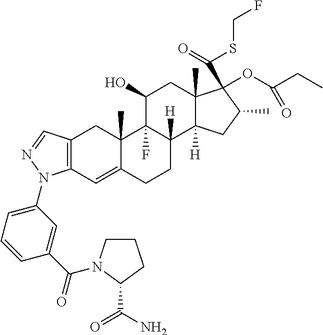 | 42 | (1R,2R,3aS,3bS,10aS,10bR,11S,12aS)- 7-(3-{[(2R)-2-carbamoylpyrrolidin-1- yl]carbonyl}phenyl)-10b-fluoro-1- {[(fluoromethyl)sulfanyl]carbonyl}- 11-hydroxy-2,10a,12a-trimethyl- 1,2,3,3a,3b,4,5,7,10,10a,10b,11,12,12a- tetradecahydrocyclopenta[5,6]naphtho [1,2-f]indazol-1-yl propanoate | 1H NMR (499.875 MHz, DMSO) δ: 7.72 (1H, s), 7.58 (4H, m), 7.52 (4H, m), 7.46 (1H, s), 7.40 (1H, s), 7.33 (1H, d), 7.30 (1H, s), 6.98 (1H, s), 6.25 (1H, m), 5.98 (1H, m), 5.88 (1H, m), 5.25 (1H, s), 4.36 (1H, m), 4.27 (1H, s), 3.60 (1H, m), 3.46 (1H, m), 3.14 (1H, m), 2.79 (1H, m), 2.24 (4H, m), 1.84 (3H, m), 1.63 (1H, m), 1.42 (1H, m), 1.38 (3H, s), 1.24 (1H, t), 1.01 (3H, s), 0.89 (3H, m). | 725.3 |
| 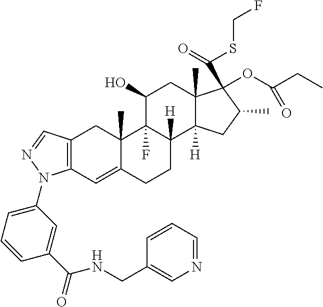 | 43 | (1R,2R,3aS,3bS,10aS,10bR,11S,12aS)- 10b-fluoro-1- {[(fluoromethyl)sulfanyl]carbonyl}-11- hydoxy-2,10a,12a-trimethyl-7-{3- [(pyridin-3- ylmethyl)carbamoyl]phenyl}- 1,2,3,3a,3b,4,5,7,10,10a,10b,11,12,12a- tetradecahydrocyclopenta[5,6]naphtho [1,2-f]indazol-1-yl propanoate | 1H NMR (499.875 MHz, DMSO) δ: 9.26 (1H, s), 8.56 (1H, d), 8.46 (1H, dd), 7.99 (1H, s), 7.90 (1H, d), 7.73 (1H, d), 7.53 (1H, s), 7.36 (2H, dd), 6.25 (1H, d), 5.98 (1H, s), 5.88 (1H, s), 5.22 (1H, d), 4.51 (2H, d), 3.14 (1H, d), 2.80 (1H, d), 2.64 (2H, t), 2.37 (5H, m), 2.15 (2H, m), 1.84 (2H, m), 1.63 (1H, m), 1.43 (1H, m), 1.32 (1H, s), 1.25 (1H, t), 1.04 (3H, t), 1.01 (3H, s), 0.90 (3H, d). | 719.5 |
| 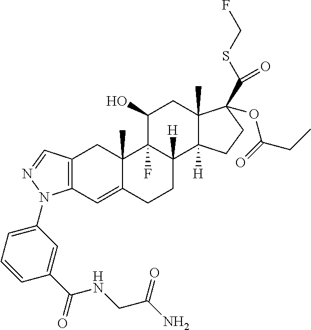 | 44 | (1R,3aS,3bS,10aS,10bR,11S,12aS)-7- {3-[(2-amino-2- oxoethyl)carbamoyl]phenyl}-10b- fluoro-1- {[(fluoromethyl)sulfanyl]carbonyl}-11- hydroxy-10a,12a-dimethyl- 1,2,3,3a,3b,4,5,7,10,10a,10b,11,12,12a- tetradecahydrocyclopenta[5,6]naphtho [1,2-f]indazol-1-yl propanoate | 1H NMR (499.875 MHz, DMSO) δ: 9.25 (1H, d), 8.56 (1H, s), 8.46 (1H, d), 7.99 (1H, s), 7.90 (1H, d), 7.74 (1H, d), 7.63 (2H, m), 7.53 (1H, s), 7.36 (1H, dd), 6.25 (1H, s), 6.00 (1H, d), 5.90 (1H, d), 5.23 (1H, d), 4.51 (2H, d), 4.17 (3H, dd), 3.13 (1H, d), 2.80 (1H, d), 2.22 (9H, m), 1.87 (1H, m), 1.64 (1H, m), 1.39 (2H, m), 1.32 (3H, s), 1.26 (1H, m), 0.99 (3H, d), 0.93 (3H, d). | 735.4 |
| 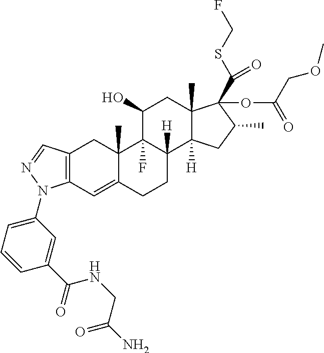 | 45 | (1R,2R,3aS,3bS,10aS,10bR,11S,12aS)- 7-{3-[(2-amino-2- oxoethyl)carbamoyl]phenyl}-10b- fluoro-1- {[(fluoromethyl)sulfanyl]carbonyl}-11- hydroxy-2,10a,12a-trimethyl- 1,2,3,3a,3b,4,5,7,10,10a,10b,11,12,12a- tetradecahydrocyclopenta[5,6]naphtho [1,2-f]indazol-1-yl methoxyacetate | 1H NMR (399.99 MHz, DMSO) δ: 8.86 (1H, m), 8.04 (1H, s), 7.89 (1H, m), 7.63 (1H, m), 7.55 (1H, s), 7.43 (1H, s), 7.07 (1H, s), 6.30 (1H, s), 6.02 (1H, m), 5.89 (1H, m), 5.23 (1H, m), 4.24 (1H, m), 4.17 (1H, d), 3.82 (1H, d), 3.14 (1H, d), 2.81 (1H, d), 2.21 (8H, m), 1.86 (3H, m), 1.63 (2H, m), 1.41 (1H, m), 1.34 (4H, s), 1.25 (2H, m), 1.03 (3H, s), 0.92 (3H, d). | 701.2 |
| 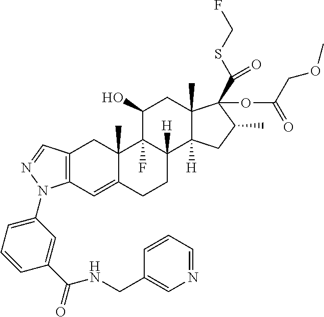 | 46 | (1R,2R,3aS,3bS,10aS,10bR,11S,12aS)- 10b-fluoro-1- {[(fluoromethyl)sulfanyl]carbonyl}-11- hydroxy-2,10a,12a-trimethyl-7-{3- [(pyridin-3- ylmethyl)carbamoyl]phenyl}- 1,2,3,3a,3b,4,5,7,10,10a,10b,11,12,12a- tetradecahydrocyclopenta[5,6]naphtho [1,2-f]indazol-1-yl methoxyacetate | 1H NMR (499.875 MHz, DMSO) δ: 9.25 (1H, d), 8.56 (1H, s), 8.46 (1H, d), 7.99 (1H, s), 7.90 (1H, d), 7.74 (1H, d), 7.63 (2H, m), 7.53 (1H, s), 7.36 (1H, dd), 6.25 (1H, s), 6.00 (1H, d), 5.90 (1H, d), 5.23 (1H, d), 4.51 (2H, d), 4.17 (3H, dd), 3.13 (1H, d), 2.80 (1H, d), 2.22 (9H, m), 1.87 (1H, m), 1.64 (1H, m), 1.39 (2H, m), 1.32 (3H, s), 1.26 (1H, m), 0.99 (3H, d), 0.93 (3H, d). | 735.4 |
| 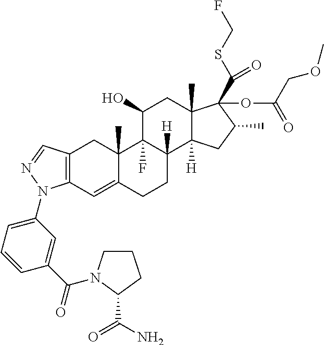 | 47 | (1R,2R,3aS,3bS,10aS,10bR,11S,12aS)- 7-(3-{[(2R)-carbamoylpyrrolidin-1- yl]carbonyl}phenyl)-10b-fluoro-1- {[(fluoromethyl)sulfanyl]carbonyl}-11- hydroxy-2,10a,12a-trimethyl- 1,2,3,3a,3b,4,5,7,10,10a,10b,11,12,12a- tetradecahydrocyclopenta[5,6]naphtho [1,2-f]indazol-1-yl methoxyacetate | 1H NMR (399.99 MHz, DMSO) δ: 7.75 (1H, s), 7.57 (2H, m), 7.52 (2H, m), 7.45 (1H, s), 7.41 (1H, s), 7.31 (1H, m), 7.19 (1H, s), 7.03 (1H, s), 6.95 (1H, m), 6.24 (1H, m), 6.01 (1H, m), 5.89 (1H, m), 5.20 (1H, s), 4.36 (1H, m), 4.21 (3H, m), 3.59 (1H, m), 3.44 (2H, m), 3.13 (1H, m), 2.79 (1H, m), 2.14 (4H, m), 1.84 (6H, m), 1.64 (1H, m), 1.41 (1H, m), 1.35 (3H, s), 1.25 (1H, m), 1.03 (3H, s), 0.93 (3H, d). | 741.4 |
| 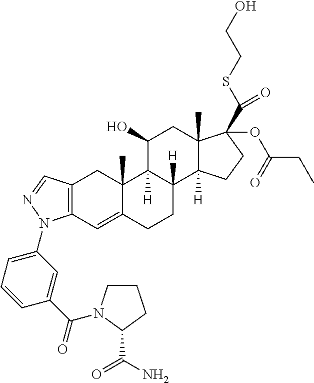 | 48 | (1R,3aS,3bS,10aR,10bS,11S,12aS)-7- (3-{[(2R)-2-carbamoylpyrrolidin-1- yl]carbonyl}phenyl)-11-hydroxy-1- {[(2-hydroxyethyl)sulfanyl]carbonyl}- 10a,12a-dimethyl- 1,2,3,3a,3b,4,5,7,10,10a,10b,11,12,12a- tetradecahydrocyclopenta[5,6]naphtho [1,2-f]indazol-1-yl propanoate | 1H NMR (400 MHz, CDCl3, TFA-d) δ 7.96-7.92 (1H, m), 7.88- 7.58 (4H, m), 6.03-6.00 (1H, m), 4.76-4.50 (2H, m), 3.96-3.60 (4H, m), 3.26-3.19 (3H, m), 3.00 (1H, m), 2.83 (1H, d), 2.65-2.54 (1H, m), 2.51-2.41 (4H, m), 2.27-1.93 (8H, m), 1.92-1.83 (1H, m), 1.74- 1.65 (1H, m), 1.56-1.47 (1H, m), 1.41-1.36 (4H, m), 1.18 (4H, t), 0.98 (3H, s). | 705 |
| 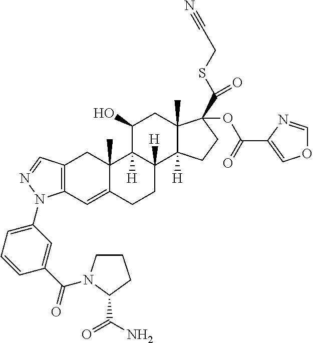 | 49 | (1R,3aS,3bS,10aR,10bS,11S,12aS)-7- (3-{[(2R)-2-carbamoylpyrrolidin-1- yl]carbonyl}phenyl)-1- {[(cyanomethyl)sulfanyl]carbonyl}-11- hydroxy-10a,12a-dimethyl- 1,2,3,3a,3b,4,5,7,10,10a,10b,11,12,12a- tetradecahydrocyclopenta[5,6]naphtho [1,2-f]indazol-1-yl 1,3-oxazole-4- carboxylate | 1H NMR (400 MHz, CDCl3, TFA-d) δ 8.48-8.46 (2H, m), 7.96- 7.92 (1H, m), 7.87-7.57 (4H, m), 6.00 (1H, s), 4.75-4.52 (2H, m), 3.89-3.60 (4H, m), 3.22 (1H, d), 3.14-3.05 (1H, m), 2.82 (1H, d), 2.67-2.56 (1H, m), 2.50-2.40 (2H, m), 2.24-1.89 (9H, m), 1.85-1.77 (1H, m), 1.63-1.54 (1H, m), 1.46 (1H, m), 1.42-1.36 (3H, m), 1.24-1.13 (1H, m), 1.08 (3H, s). | 739 |
| 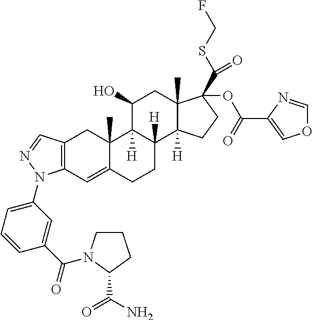 | 50 | (1R,3aS,3bS,10aR,10bS,11S,12aS)-7- (3-{[(2R)-2-carbamoylpyrrolidin-1- yl]carbonyl}phenyl)-1- {[(fluoromethyl)sulfanyl]carbonyl}-11- hydroxy-10a,12a-dimethyl- 1,2,3,3a,3b,4,5,7,10,10a,10b,11,12,12a- tetradecahydrocyclopenta[5,6]naphtho [1,2-f]indazol-1-yl 1,3-oxazole-4- carboxylate | 1H NMR (400 MHz, CDCl3, TFA-d) δ 8.48 (2H, s), 7.96-7.93 (1H, m), 7.87-7.57 (4H, m), 6.02- 5.70 (3H, m), 4.73 (1H, t), 4.65 (1H, s), 3.91-3.61 (2H, m), 3.25- 3.10 (2H, m), 2.82 (1H, d), 2.65- 2.55 (1H, m), 2.49-2.41 (2H, m), 2.37-2.30 (1H, m), 2.27-1.99 (5H, m), 1.97-1.89 (1H, m), 1.87-1.78 (1H, m), 1.62-1.52 (1H, m), 1.52- 1.46 (1H, m), 1.44-1.36 (4H, m), 1.26-1.14 (1H, m), 1.06 (3H, s). | 732 |
| 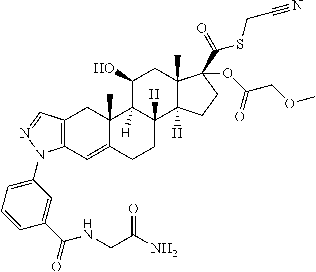 | 51 | (1R,3aS,3bS,10aR,10bS,11S,12aS)-7- {3-[(2-amino-2- oxoethyl)carbamoyl]phenyl}-1- {[(cyanomethyl)sulfanyl]carbonyl}-11- hydroxy-10a,12a-dimethyl- 1,2,3,3a,3b,4,5,7,10,10a,10b,11,12,12a- tetradecahydrocyclopenta[5,6]naphtho [1,2-f]indazol-1-yl methoxyacetate | 1H NMR (400.0 MHz, CD3CN) δ 7.95 (1H, t), 7.84-7.80 (1H, m), 7.70-7.65 (1H, m), 7.61 (1H, dd), 7.43-7.48 (2H, m), 6.37 (1H, s), 6.22 (1H, d), 5.78 (1H, s), 4.50- 4.46 (1H, m), 4.05 (2H, d), 3.94 (2H, d), 3.75 (2H, s), 3.38 (3H, s), 2.99 (1H, d), 2.90 (1H, ddd), 2.70 (1H, d), 2.63 (1H, d), 2.51 (1H, m), 2.32 (1H, d), 2.08-1.97 (3H, m), 1.85-1.76 (1H, m), 1.74- 1.64 (1H, m), 1.50-1.38 (1H, m), 1.30-1.27 (3H, m), 1.22 (1H, dd), 1.06 (1H, ddd), 0.96 (3H, s). | 677 |
| 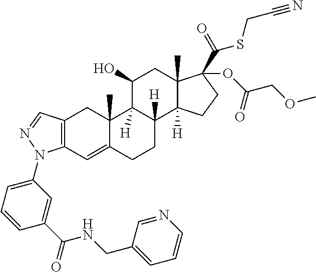 | 52 | (1R,3aS,3bS,10aR,10bS,11S,12aS)-1- {[(cyanomethyl)sulfanyl]carbonyl}-11- hydroxy-10a,12a-dimethyl-7-{3- [(pyridin-3- ylmethyl)carbamoyl]phenyl}- 1,2,3,3a,3b,4,5,7,10,10a,10b,11,12,12a- tetradecahydrocyclopenta[5,6]naphtho [1,2-f]indazol-1-yl methoxyacetate | 1H NMR (400 MHz, CD3CN) δ 8.59 (1H, d), 8.47 (1H, dd), 7.93 (1H, t), 7.80 (1H, dt), 7.75 (1H, dt), 7.67 (2H, m), 7.59 (2H, t), 7.43 (1H, s), 7.31 (1H, dd), 6.20 (1H, d), 4.58 (2H, d), 4.47 (1H, m), 4.05 (2H, d), 3.75 (2H, s), 3.37 (3H, s), 2.99 (1H, d), 2.90 (1H, ddd), 2.69 (1H, d), 2.64 (1H, d), 2.51 (1H, m), 2.31 (1H, m), 2.18-1.98 (3H, m), 1.81 (1H, m), 1.69 (1H, m), 1.44 (1H, m), 1.28 (3H, s), 1.22 (1H, dd), 1.13-0.98 (1H, m), 0.95 (3H, s). | 710 |
| 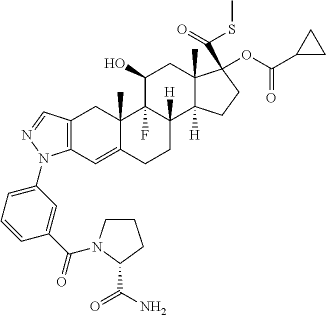 | 53 | (1R,3aS,3bS,10aS,10bR,11S,12aS)-7- (3-{[(2R)-2-carbamoylpyrrolidin-1- yl]carbonyl}phenyl)-10b-fluoro-11- hydroxy-10a,12a-dimethyl-1- [(methylsulfanyl)carbonyl]- 1,2,3,3a,3b,4,5,7,10,10a,10b,11,12,12a- tetradecahydrocyclopenta[5,6]naphtho [1,2-f]indazol-1-yl cyclopropane- carboxylate | 1H NMR (400.0 MHz, CDCl3) δ 7.68 (1H, s), 7.62-7.47 (4H, m), 6.91 (1H, s), 6.24 (1H, d), 5.37 (1H, s), 4.81 (1H, dd), 4.48 (1H, d), 3.66-3.50 (2H, m), 3.35 (1H, d), 3.05-2.95 (1H, m), 2.81 (1H, d), 2.60 (1H, m), 2.49 (2H, m), 2.40- 2.29 (5H, m), 2.27-1.58 (11H, m), 1.43 (4H, m), 1.25 (3H, d), 1.14-1.01 (2H, m), 0.94 (3H, s), 0.92 (2H, m). | 705 |
| 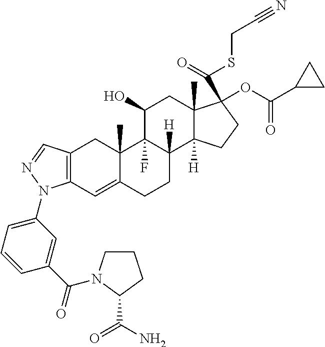 | 54 | (1R,3aS,3bS,10aS,10bR,11S,12aS)-7- (3-{[(2R)-2-carbamoylpyrrolidin-1- yl]carbonyl}phenyl)-1- {[(cyanomethyl)sulfanyl]carbonyl}- 10b-fluoro-11-hydroxy-10a,12a- dimethyl-1,2,3,3a,3b,4,5,7,10,10a,10b, 11,12,12a-tetradecahydrocyclopenta [5,6]naphtho[1,2-f]indazol-1-yl cyclopropanecarboxylate | 1H NMR (400.0 MHz, CDCl3) δ 7.68 (1H, s), 7.54 (4H, m), 6.91 (1H, s), 6.24 (1H, d), 5.40 (1H, s), 4.80 (1H, dd), 4.49 (1H, d), 3.80 (1H, d), 3.67-3.49 (3H, m), 3.34 (1H, d), 3.02-2.92 (1H, m), 2.81 (1H, d), 2.60 (1H, m), 2.50 (2H, m), 2.40-1.44 (16H, m), 1.41 (3H, s), 1.06 (2H, m), 1.00 (3H, s), 0.95 (3H, dd). | 730 |
| 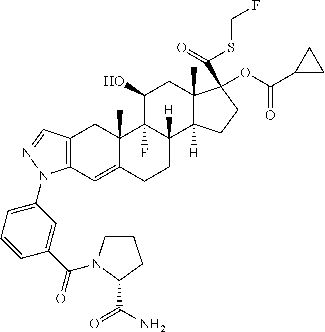 | 55 | (1R,3aS,3bS,10aS,10bR,11S,12aS)-7- (3-{[(2R)-2-carbamoylpyrrolidin-1- yl]carbonyl}phenyl)-10b-fluoro-1- {[(fluoromethyl)sulfanyl]carbonyl}-11- hydroxy-10a,12a-dimethyl- 1,2,3,3a,3b,4,5,7,10,10a,10b,11,12,12a- tetradecahydrocyclopenta[5,6]naphtho [1,2-f]indazol-1-yl cyclopropane- carboxylate | 1H NMR (400.0 MHz, CDCl3) δ 7.68 (1H, s), 7.54 (4H, m), 6.91 (1H, s), 6.24 (1H, d), 5.99 (1H, dd), 5.69 (1H, dd), 5.41 (1H, s), 4.80 (1H, dd), 4.47 (1H, d), 3.66-3.50 (2H, m), 3.34 (1H, d), 3.04-2.94 (1H, m), 2.89 (1H, s), 2.80 (1H, d), 2.60 (1H, m), 2.53-2.44 (2H, m), 2.39-1.53 (16H, m), 1.48-1.36 (5H, m), 1.22 (2H, t), 1.12-1.01 (2H, m), 0.98 (3H, s), 0.93 (3H, dd). | 723 |
| 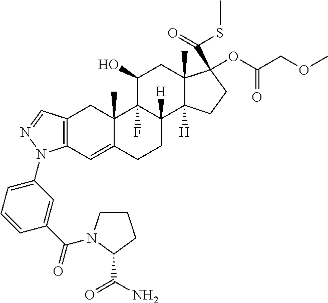 | 56 | (1R,3aS,3bS,10aS,10bR,11S,12aS)-7- (3-{[(2R)-2-carbamoylpyrrolidin-1- yl]carbonyl}phenyl)-10b-fluoro-11- hydroxy-10a,12a-dimethyl-1- [(methylsulfanyl)carbonyl]- 1,2,3,3a,3b,4,5,7,10,10a,10b,11,12,12a- tetradecahydrocyclopenta[5,6]naphtho [1,2-f]indazol-1-yl methoxyacetate | 1H NMR (400.0 MHz, CDCl3) δ 7.68 (1H, s), 7.53 (4H, ddd), 6.92 (1H, s), 6.23 (1H, d), 5.42 (1H, s), 4.80 (1H, dd), 4.46 (1H, d), 4.07 (2H, dd), 3.67-3.50 (2H, m), 3.47 (3H, s), 3.33 (1H, d), 3.11-3.01 (1H, m), 2.79 (1H, d), 2.61 (1H, m), 2.48 (2H, m), 2.37 (1H, m), 2.33 (3H, s), 2.30-2.02 (6H, m), 1.93-1.43 (6H, m), 1.40 (3H, s), 1.32 (1H, s), 0.95 (3H, s). | 709 |
| 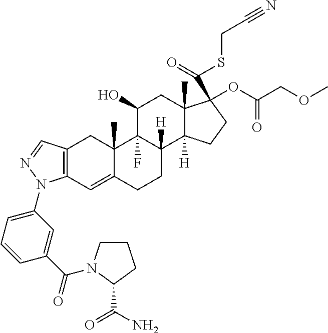 | 57 | (1R,3aS,3bS,10aS,10bR,11S,12aS)-7- (3-{[(2R)-2-carbamoylpyrrolidin-1- yl]carbonyl}phenyl)-1- {[(cyanomethyl)sulfanyl]carbonyl}- 10b-fluoro-11-hydroxy-10a,12a- dimethyl- 1,2,3,3a,3b,4,5,7,10,10a,10b,11,12,12a- tetradecahydrocyclopenta[5,6]naphtho [1,2-f]indazol-1-yl methoxyacetate | 1H NMR (400.0 MHz, CDCl3) δ 7.68 (1H, s), 7.61-7.46 (5H, m), 6.90 (1H, s), 6.23 (1H, d), 5.41 (1H, s), 4.80 (1H, dd), 4.47 (1H, d), 4.15-4.02 (2H, m), 3.82 (1H, d), 3.67-3.50 (3H, m), 3.47 (3H, s), 3.32 (1H, d), 3.08-2.98 (1H, m), 2.80 (1H, d), 2.61 (1H, dd), 2.52-2.02 (10H, m), 1.93- 1.69 (5H, m), 1.65-1.44 (9H, m), 1.41 (3H, s), 1.01 (3H, s). | 734 |
| 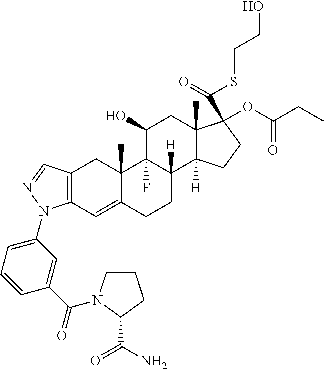 | 58 | (1R,3aS,3bS,10aS,10bR,11S,12aS)-7- (3-{[(2R)-2-carbamoylpyrrolidin-1- yl]carbonyl}phenyl)-10b-fluoro-11- hydroxy-1-{[(2- hydroxyethyl)sulfanyl]carbonyl}- 10a,12a-dimethyl- 1,2,3,3a,3b,4,5,7,10,10a,10b,11,12,12a- tetradecahydrocyclopenta[5,6]naphtho [1,2-f]indazol-1-yl propanoate | 1H NMR (400.0 MHz, CDCl3) δ 7.68 (1H, s), 7.61-7.45 (4H, m), 6.91 (1H, s), 6.23 (1H, d), 5.43 (1H, s), 4.79 (1H, dd), 4.46 (1H, d), 3.77 (2H, t), 3.59 (2H, ddt), 3.32 (1H, d), 3.21-3.05 (2H, m), 3.05-2.95 (1H, m), 2.79 (1H, d), 2.66-1.42 (17H, m), 1.40 (3H, s), 1.16 (3H, t), 0.96 (3H, s). | 723 |
| 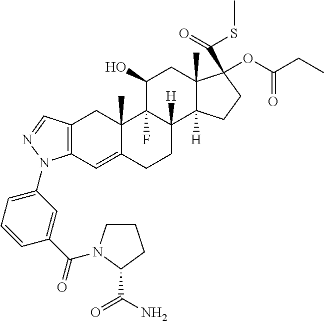 | 59 | (1R,3aS,3bS,10aS,10bR,11S,12aS)-7- (3-{[(2R)-2-carbamoylpyrrolidin-1- yl]carbonyl}phenyl)-10b-fluoro-11- hydroxy-10a,12a-dimethyl-1- [(methylsulfanyl)carbonyl]- 1,2,3,3a,3b,4,5,7,10,10a,10b,11,12,12a- tetradecahydrocyclopenta[5,6]naphtho [1,2-f]indazol-1-yl propanoate | 1H NMR (400.0 MHz, CDCl3) δ 7.68 (1H, s), 7.61-7.45 (45H, m), 6.92 (1H, s), 6.23 (1H, d), 5.43 (1H, s), 4.80 (1H, dd), 4.46 (1H, d), 3.66-3.49 (2H, m), 3.33 (1H, d), 3.07-2.97 (1H, m), 2.80 (1H, d), 2.61 (1H, m), 2.54-1.50 (16H, m), 1.40 (3H, s), 1.37 (1H, m), 1.16 (3H, t), 0.94 (3H, s). | 693 |
| 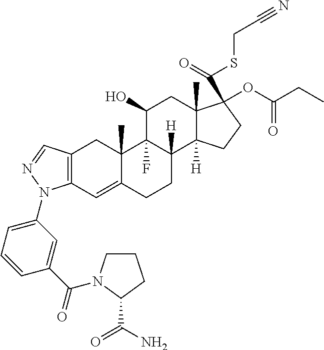 | 60 | (1R,3aS,3bS,10aS,10bR,11S,12aS)-7- (3-{[(2R)-2-carbamoylpyrrolidin-1- yl]carbonyl}phenyl-1- {[(cyanomethyl)sulfanyl]carbonyl}- 10b-fluoro-11-hydroxy-10a,12a- dimethyl-1,2,3,3a,3b,4,5,7,10,10a,10b, 11,12,12a-tetradecahydrocyclopenta [5,6]naphtho[1,2-f]indazol-1-yl propanoate | 1H NMR (400.0 MHz, CDCl3) δ 7.68 (1H, s), 7.61-7.46 (4H, m), 6.91 (1H, s), 6.23 (1H, d), 5.43 (1H, s), 4.80 (1H, dd), 4.47 (1H, d), 3.81 (1H, d), 3.67-3.50 (3H, m), 3.32 (1H, d), 3.04-2.93 (1H, m), 2.80 (1H, d), 2.67-2.55 (1H, m), 2.52-1.42 (16H, m), 1.41 (3H, s), 1.16 (3H, t), 1.00 (3H, s). | 718 |
| 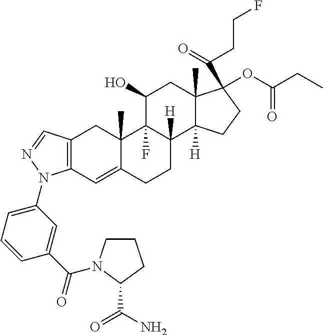 | 61 | (1R,3aS,3bS,10aS,10bR,11S,12aS)-7- (3-{[(2R)-2-carbamoylpyrrolidin-1- yl]carbonyl}phenyl)-10b-fluoro-1- {[(fluoromethyl)sulfanyl]carbonyl}-11- hydroxy-10a,12a-dimethyl- 1,2,3,3a,3b,4,5,7,10,10a,10b,11,12,12a- tetradecahydrocyclopenta[5,6]naphtho [1,2-f]indazol-1-yl propanoate | 1H NMR (400.0 MHz, CDCl3) δ 7.67 (1H, m), 7.61-7.44 (4H, m), 6.91 (1H, s), 6.23 (1H, s), 5.99 (1H, dd), 5.69 (1H, dd), 5.42 (1H, s), 4.82-4.76 (1H, m), 4.47 (1H, s), 3.67-3.49 (2H, m), 3.33 (1H, d), 3.05-2.96 (1H, m), 2.80 (1H, d), 2.60 (1H, m), 2.54-1.42 (16H, m), 1.40 (3H, s), 1.17 (3H, t), 0.99 (3H, s). | 711 |
| 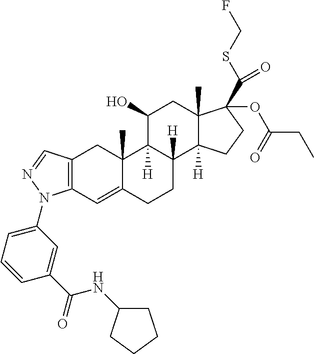 | 62 | (1R,3aS,3bS,10aR,10bS,11S,12aS)-7- [3-(cyclopentylcarbamoyl)phenyl]-1- {[(fluoromethyl)sulfanyl]carbonyl}-11- hydroxy-10a,12a-dimethyl- 1,2,3,3a,3b,4,5,7,10,10a,10b,11,12,12a- tetradecahydrocyclopenta[5,6]naphtho [1,2-f]indazol-1-yl propanoate | 1H NMR (400 MHz, CD3CN) δ 7.90-7.88 (1H, m), 7.77 (1H, d), 7.64-7.54 (2H, m), 7.45 (1H, s), 7.03-6.98 (1H, m), 6.19 (1H, d), 5.91 (1H, m), 5.78 (1H, m), 4.50- 4.46 (1H, m), 4.36-4.30 (1H, m), 3.00 (1H, d), 2.91-2.83 (1H, m), 2.71 (1H, d), 2.56-2.46 (1H, m), 2.39-2.28 (3H, m), 2.13-1.98 (2H, m), 1.83-1.53 (8H, m), 1.48-1.37 (1H, m), 1.29 (3H, s), 1.27-1.23 (1H, m), 1.10 (3H, t), 1.06-1.00 (1H, m), 0.94 (3H, s). | 664 |

### Human Glucocorticoid Receptor (GR) Assay

The assay is based on a commercial kit from Panvera/Invitrogen (Part number P2893). The assay technology is fluorescence polarization. The kit utilises recombinant human GR (Panvera, Part number P2812), a Fluoromone™ labelled tracer (GS Red, Panvera, Part number P2894) and a Stabilizing Peptide 10× (Panvera, Part number P2815). The GR and Stabilizing Peptide reagents are stored at −70° C. while the GS Red is stored at −20° C. Also included in the kit are 1M DTT (Panvera, Part number P2325, stored at −20° C.) and GR Screening buffer 10× (Panvera, Part number P2814, stored at −70° C. initially but once thawed stored at room temperature). Avoid repeated freeze/thaws for all reagents. The GR Screening buffer 10× comprises 100 mM potassium phosphate, 200 mM sodium molybdate, 1 mM EDTA and 20% DMSO.

Test compounds (1 μL) and controls (1 μL) in 100% DMSO were added to black polystyrene 384-well plates (Greiner low volume black flat-bottom, part number 784076). 0% control was 100% DMSO and 100% control was 10 μM Dexamethasone. Background solution (8 L; assay buffer 10×, Stabilizing Peptide, DTT and ice cold MQ water) was added to the background wells. GS Red solution (74; assay buffer 10×, Stabilizing Peptide, DTT, GS Red and ice cold water) was added to all wells except background wells. GR solution (7 μL; assay buffer 10×, Stabilizing Peptide, DTT, GR and ice cold water) was added to all wells. The plate was sealed and incubated in a dark at room temperature for 2 hours. The plate was read in an Analyst plate reader (LJL Biosystems/Molecular Devices Corporation) or other similar plate reader capable of recording fluorescence polarization (excitation wavelength 530 nm, emission wavelength 590 nm and a dichroic mirror at 561 nm). The IC₅₀ values were calculated using XLfit model 205 and are shown in Table 1.

TABLE 1

| | Inhibition of GR | | Inhibition of GR |
|---|---|---|---|
| Example No. | binding, IC₅₀ (nM) | Example No. | binding, IC₅₀ (nM) |
| 1 | 1 | 2 | 0.81 |
| 3 | 1.6 | 4 | 0.91 |
| 5 | 0.98 | 6 | 1.7 |
| 7 | 1.4 | 8 | 1.8 |
| 9 | 0.91 | 10 | 1.5 |
| 11 | 3.7 | 12 | 4.3 |
| 13 | 2.5 | 14 | 6 |
| 15 | 4.3 | 16 | 9 |
| 17 | 3 | 18 | 1.4 |
| 22 | 1.9 | 23 | 4 |
| 24 | 2.1 | 25 | 2.8 |
| 26 | 2 | 27 | 0.47 |
| 28 | 2.3 | 29 | 2.6 |
| 30 | 2.2 | 31 | 1.2 |
| 32 | 2.5 | 33 | 5.2 |
| 34 | 3.8 | 35 | 6.5 |
| 36 | 0.84 | 37 | 7.1 |
| 38 | 8.3 | 39 | 2.4 |
| 40 | 3 | 41 | 1.2 |
| 42 | 4.4 | 43 | 1.9 |
| 44 | 2.2 | 45 | 5.3 |
| 46 | 3.3 | 47 | 7.5 |
| 48 | 6.3 | 49 | 12 |
| 50 | 8.2 | 51 | 2.8 |
| 52 | 1.5 | 53 | 2.5 |
| 54 | 4.2 | 55 | 3.5 |
| 56 | 8.6 | 57 | 10 |
| 58 | 6.9 | 59 | 3.3 |
| 60 | 3.3 | 61 | 3.6 |
| 62 | 3.6 | | |

1-10. (canceled)

11. A compound, wherein the compound is:
(1R,3aS,3bS,10aR,10bS,11S,12aS)-1-{[(Cyanomethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aR,10bS,11S,12aS)-1-{[(Fluoromethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aR,10bS,11S,12aS)-1-{[(Fluoromethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazol-1-yl methoxyacetate,
(1R,3aS,3bS,10aR,10bS,11S,12aS)-1-{[(Cyanomethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazol-1-yl cyclopropanecarboxylate,
(1R,3aS,3bS,10aR,10bS,11S,12aS)-1-{[(Fluoromethyl)sulfanyl]carbonyl}-11-hydroxy-7-{3-[(2-methoxyethyl)carbamoyl]phenyl}-10a,12a-dimethyl-1,2,3,3a,3b,4,5,7,10,10a,10b,11,12,12a-tetradecahydrocyclopenta[5,6]naphtho[1,2-f]indazol-1-yl cyclopropanecarboxylate,
(1R,3aS,3bS,10aR,10bS,11S,12aS)-1-{[(Cyanomethyl)sulfanyl]carbonyl}-11-hydroxy-10a,12a-dimethyl-7-(3-{[2-(methylsulfanyl)ethyl]carbamoyl}phenyl)-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aR,10bS,11S,12aS)-1-{[(Fluoromethyl)sulfanyl]carbonyl}-11-hydroxy-10a,12a-dimethyl-7-(3-{[2-(methylsulfanyl)ethyl]carbamoyl}phenyl)-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aR,10bS,11S,12aS)-1-{[(Fluoromethyl)sulfanyl]carbonyl}-11-hydroxy-10a,12a-dimethyl-7-(3-{[2-(methylsulfanyl)ethyl]carbamoyl}phenyl)-1,2,3,3a,3b,4,5,7,10,10a,10b,11,12,12a-tetradecahydrocyclopenta[5,6]naphtho[1,2-f]indazol-1-yl methoxyacetate,
(1R,3aS,3bS,10aR,10bS,11S,12aS)-1-{[(Cyanomethyl)sulfanyl]carbonyl}-11-hydroxy-10a,12a-dimethyl-7-(3-{[2-(methylsulfanyl)ethyl]carbamoyl}phenyl)-1,2,3,3a,3b,4,5,7,10,10a,10b,11,12,12a-tetradecahydrocyclopenta[5,6]naphtho[1,2-f]indazol-1-yl cyclopropanecarboxylate,
(1R,3aS,3bS,10aR,10bS,11S,12aS)-1-{[(Fluoromethyl)sulfanyl]carbonyl}-11-hydroxy-10a,12a-dimethyl-7-(3-{[2-(methylsulfanyl)ethyl]carbamoyl}phenyl)-1,2,3,3a,3b,4,5,7,10,10a,10b,11,12,12a-tetradecahydrocyclopenta[5,6]naphtho[1,2-f]indazol-1-yl cyclopropanecarboxylate,
(1R,3aS,3bS,10aR,10bS,11S,12aS)-7-{3-[(2-Amino-2-oxoethyl)carbamoyl]phenyl}-1-{[(cyanomethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aR,10bS,11S,12aS)-1-{[(Cyanomethyl)sulfanyl]carbonyl}-7-{3-[(1,1-dioxidotetrahydrothiophen-3-yl)carbamoyl]phenyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aR,10bS,11S,12aS)-7-(3-{[(2R)-2-Carbamoylpyrrolidin-1-yl]carbonyl}phenyl)-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate,
(1R,3aS,3bS,10aR,10bS,11S,12aS)-7-(3-{[(2R)-2-Carbamoylpyrrolidin-1-yl]carbonyl}phenyl)-1-{[(cyanomethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl propanoate, or
(1R,3aS,3bS,10aS,10bR,11S,12aS)-7-(3-{[(2R)-2-Carbamoylpyrrolidin-1-yl]carbonyl}phenyl)-10b-fluoro-1-{[(fluoromethyl)sulfanyl]carbonyl}-11-hydroxy-10a,12a-dimethyl-1,2,3,3a,3b,4,5,7,10,10a,10b,11,12,12a-tetradecahydrocyclopenta[5,6]naphtho[1,2-f]indazol-1-yl methoxyacetate,
or
a pharmaceutically acceptable salt thereof.

12. (canceled)

13. A pharmaceutical composition comprising:
a compound as claimed in claim 11 or a pharmaceutically acceptable salt thereof and
a pharmaceutically acceptable adjuvant, diluent or carrier.

14. (canceled)
